(19) United States
(12) Patent Application Publication
(10) Pub. No.: US 2026/0137859 A1
(43) Pub. Date: May 21, 2026

(54) FLUIDICS CONTROL SYSTEM FOR MULTI CATHETER STACK

(71) Applicant: Imperative Care, Inc., Campbell, CA (US)

(72) Inventors: Kyle Bartholomew, Campbell, CA (US); Tabish Mustufa, Sunnyvale, CA (US); Vedant Chhaya, Campbell, CA (US)

(21) Appl. No.: 19/394,754

(22) Filed: Nov. 19, 2025

Related U.S. Application Data
(60) Provisional application No. 63/722,945, filed on Nov. 20, 2024.

(51) Int. Cl. A61M 5/00; A61M 1/00; A61M 5/14; A61M 5/142; A61M 5/168; A61M 5/36; A61M 25/01

(52) U.S. Cl. CPC A61M 5/007; A61M 1/85; A61M 5/1408; A61M 5/14228; A61M 5/16813; A61M 5/1689; A61M 25/0116; A61M 5/365; A61M 2205/3306; A61M 2205/3375; A61M 2205/3379

(57) ABSTRACT

A robotic catheter system having fluidics system including a pump station interface with a contrast pump actuator, a camera, a peristaltic pump actuator, and an electrical interface, and a cassette having a distal side and a proximal side, the proximal side of the cassette configured to releasably couple to the pump station interface, the cassette comprising a contrast pump, a saline channel configured to interface with the peristaltic pump actuator, and a drip chamber and clot pod positioned in the cassette to be adjacent to the camera when the cassette is coupled to the interface.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57. The present application claims priority to, and the benefit of, U.S. Provisional Patent Application No. 63/722,945, filed Nov. 20, 2024, titled FLUIDICS CONTROL SYSTEM FOR MULTI CATHETER STACK. The present application is related to U.S. Provisional Patent Application No. 63/467,251, filed May 17, 2023, titled FLUIDICS CONTROL SYSTEM FOR MULTI CATHETER STACK, U.S. Provisional Patent Application No. 63/528,038, filed Jul. 20, 2023, titled FLUIDICS CONTROL SYSTEM FOR MULTI CATHETER STACK, U.S. Provisional Patent Application No. 63/550,926, filed Feb. 7, 2024, titled FLUIDICS CONTROL SYSTEM FOR MULTI CATHETER STACK, and U.S. patent application Ser. No. 18/666,217, filed May 16, 2024, titled FLUIDICS CONTROL SYSTEM FOR MULTI CATHETER STACK, the entire content of each of which is incorporated by reference herein for all purposes and forms a part of this specification.

### BACKGROUND

### Field

This disclosure relates generally to the field of fluidics infrastructure, and more specifically to the field of fluid management and delivery during medical procedures, either manual or robotically driven. Described herein are systems and methods for fluidics management and delivery.

### Description of the Related Art

Any of a variety of endoluminal or endovascular medical procedures may involve introduction of a number of tools such as catheters into the body either simultaneously or sequentially. Each catheter may require a unique connection to any of a variety of sources of aspiration, irrigation, drug, saline or contrast infusion. Such sources are conventionally placed in communication with the catheter via tubing ending in a connector for releasable connection to a complementary port on a catheter hub (mount).

A catheter exchange typically involves disconnecting tubing from a first catheter being removed and reconnecting the tubing to a second, replacement catheter. In addition, catheters typically have one luer connection port for injection of all fluids as well as for aspiration. During the course of a procedure, multiple different fluids and/or fluid volumes may be injected at different times in addition to aspiration. As such, fluid sources such as syringes are frequently connected and disconnected from the luer connection port. This conventional switching of components, syringes, and fluidic connections during a procedure can lead to a risk of air bubble introduction, errors at connection points, and/or errors in fluid selection.

Thus, there remains a need for an improved fluid and tool management system that overcomes one or more of the drawbacks of conventional fluid management and catheter exchange systems.

### SUMMARY

A robotic catheter system having an aspiration system with integrated fluidics management. The robotic catheter system can include a remotely located controller, a local controller, a drive table, a pump station, a cassette (or cartridge) that is releasably couplable to the pump station, fluidic channels for saline, contrast, and vacuum, and one or more hub assemblies structured to be magnetically driven by the drive table without a mechanical engagement between the drive table and the one or more hub assemblies. An elongate, flexible tubular body having a proximal end, a distal end and at least one lumen can be coupled to each hub assembly. The fluidics management system may be used with a two or three or four or more interventional device stack (e.g., concentrically mounted catheters over a guidewire) for either a manually operated or robotically driven intervention.

One innovation includes a fluidics system, comprising a cassette (“cartridge”) having a housing with a proximal surface structured to be releasably coupled to a pump station. The cassette can include a vacuum subsystem having a vacuum flow-path for communicating aspirated material, the vacuum subsystem including a drip pod positioned in the vacuum flow path. The drip pod can include a drip chamber structured to allow observation of an amount of fluid (e.g., from a catheter) being communicated through the vacuum flow-path, and can include a clot pod structured to collect material communicated through the vacuum flow-path. The clot pod can be coupled to the drip chamber. The drip pod advantageously includes a transparent wall adjacent to the cassette proximal surface for allowing an imaging system to image material in the drip chamber and the clot pod through the transparent wall. The clot pod can be rectangular-shaped, circular-shaped, or have another shape based on the implementation. The clot pod includes an inlet port and an outlet port, and a screen positioned between the inlet port and outlet port for collecting material communicated through the clot pod. Various embodiments can have additional features. In some embodiments, the screen can be positioned at an angle in the clot pod such that a first portion of the screen is farther from the proximal surface and a second portion of the screen is located closer to the proximal surface of the clot pod. In some embodiments, the screen is positioned at an angle in the clot pod such that a first portion of the screen is farther from the proximal surface than the inlet port and a second portion of the screen is located closer to the proximal surface than the outlet port. The system can further include the pump station, wherein the pump station and the cassette are configured such that the drip pod is positioned at an angle of between about 30 degrees and 80 degrees above horizontal, with the inlet port positioned higher than the outlet port, when the cassette is coupled to the pump station interface. In some embodiments, the cassette is configured such that the drip pod is positioned at an angle of between about 30 degrees and about 80 degrees above horizontal, when the proximal surface of the cassette is coupled to the pump station. In some embodiments, the cassette is configured such that the drip pod is positioned at an angle of about 45 degrees above horizontal, when the proximal surface of the cassette is coupled to the pump station. The drip chamber can be structured to include a first portion and a second portion, the second portion being shallower than the first portion. The second portion can be defined by a shelf extending from a side of the drip chamber into the drip chamber to create the second portion of the drip chamber. In some embodiments, the drip chamber further includes graduations disposed between the second portion of the drip chamber and the transparent wall, the graduations indicative of a level of fluid in the drip chamber. In some embodiments, the cassette includes a proximal side that includes the proximal surface and a distal side, opposite the proximal side, the cassette further including masking positioned to obscure a portion of components inside the cassette when the cassette is viewed from its distal side. The cassette can include a contrast pump configured to interface with a contrast pump actuator on the pump station when the cassette is coupled to the pump station. The cassette can further includes a saline subsystem and a contrast system, each coupled to the vacuum subsystem, the saline and contrast subsystems configured to selectively communicate saline and contrast to the vacuum subsystem.

Another innovation includes a fluidics system having a pump station interface comprising a contrast pump actuator, a camera, a peristaltic pump actuator, and an electrical interface; and a cassette having a distal side and a proximal side, the proximal side of the cassette configured to releasably couple to the pump station interface, the cassette comprising a contrast pump, a saline channel configured to interface with the peristaltic pump actuator, and a drip chamber and clot pod positioned in the cassette to be adjacent to the camera when the cassette is coupled to the interface. The drip chamber can include a main compartment and a shelf extending from the side of the drip chamber into the main compartment to create a shallower portion of the drip chamber adjacent to the proximal side of the cassette. The drip chamber can further include graduations indicative of a level of fluid in the drip chamber. In some embodiments, the fluidics system further includes a drip pod comprising the drip chamber and the clot pod, wherein the drip pod is positioned at an angle of between about 30 degrees and 60 degrees relative to vertical when the cassette is coupled to the pump station interface. In some embodiments, the drip chamber is positioned at an angle of about 45 degrees relative to vertical when the cassette is coupled to the pump station interface. The clot pod can include a screen positioned at an angle within the clot pod to form a clot collection portion of the clot pod that is shallower on one side of the clot pod than the other side of the clot pod. In some embodiments, the clot pod is configured such that the clot collection portion of the clot pod is shallower on a lower portion of the clot pod than a upper portion of the clot pod when the cassette is coupled to the pump station interface. In some embodiments, at least a portion of the surfaces of the cassette are transparent which advantageously allows a user to inspect certain fluidic components (e.g., for air bubbles). In some embodiments, the cassette includes masking positioned to obscure a portion of components inside the cassette when the cassette is viewed from its distal side. In some embodiments, the contrast pump actuator is configured to receive the contrast pump when the cassette is coupled to the pump station interface, and wherein the contrast pump actuator includes at least one piston rod attached to a latch assembly that couples to a portion of the contrast pump, the contrast pump actuator configured to actuate the contrast pump to provide contrast at high pressure to a contrast flowpath by pulling the latch with the at least one piston rod. In some embodiments, the pump station interface further comprises a plurality of control valve actuators, and wherein the cassette further comprises a plurality of control valves positioned to correspond the plurality of control valve actuators, wherein the plurality of control valves are configured to selectively control the flow of contrast from the contrast pump through the cassette and selectively control the flow of saline through the saline channel of the cassette.

Another innovation includes a method of remotely controlling a robotic catheter system to aspirate material, the robotic catheter system providing one or more liquids and vacuum from a cassette releasably coupled to a pump station to a plurality of catheters, each catheter coupled to one of a plurality of hub assemblies in fluid communication, the method comprising providing vacuum to a selected one or more catheters of the plurality of catheters; communicating material through the selected one or more catheters through a vacuum flow-path into a drip pod comprising a drip chamber and a clot pod, the drip chamber having a first portion and a second portion, the second portion being shallower than the first portion, the drip chamber including graduations positioned over the second portion to indicate a fluid level in the drip chamber; capturing images depicting material in the drip pod with an imaging system; and displaying the captured images at a remotely located control system, wherein the displayed images show a fluid level in the drip chamber and the graduations positioned over the second portion of the drip chamber for indicating the fluid level in the drip chamber. Capturing images depicting material in the drip pod includes capturing images depicting material collected in the clot pod by a screen (or filter). In some embodiments, the method can further include receiving a control signal from the remotely located control system to control a clot aspiration procedure performed by the robotic catheter system after displaying the captured images at the remotely located control system. In some embodiments, capturing images depicting material in the drip pod comprises capturing images depicting the amount of fluid collected in the drip chamber. The method can further include receiving a control signal from the remotely located control system to control a back-bleeding procedure of the robotic catheter system after displaying the captured images depicting the amount of fluid collected in the drip chamber.

Another innovation includes a method of remotely controlling a robotic catheter system to aspirate material, the robotic catheter system providing one or more liquids and vacuum from a cassette releasably coupled to a pump station to a plurality of catheters, each catheter coupled to one of a plurality of hub assemblies in fluid communication with the cassette, the method including providing vacuum to a selected one or more catheters of the plurality of catheters, communicating material through the selected one or more catheters through a vacuum flow-path into a drip chamber, capturing images depicting material in a drip chamber through a transparent wall of the drip chamber with an imaging system, and communicating the captured images to a remotely located control system for displaying a fluid level in the drip chamber. In some embodiments, the method further includes receiving a control signal from the remotely located control system at a local control system to stop communicating material through the selected one or more catheters and through the vacuum flow-path into the drip chamber. The drip chamber can include a first portion and a second portion, the second portion being shallower than the first portion and the second portion, and further includes graduations positioned over the second portion to indicate a fluid level in the drip chamber. As illustrated herein, a drip chamber and a clot pod can be included in a cassette. In some embodiments, a drip chamber and/or a clot pod are not included in the cassette. In some embodiments, a method can further include capturing images depicting material collected in a clot pot through a transparent wall of the clot pod with the imaging system, and communicating the captured images to the remotely located control system for displaying the material collected in the clot pod. In some embodiments, the method further includes after communicating the captured images to the remotely located control system, receiving at a local system controller, from the remotely located control system, a control signal to control a clot aspiration procedure performed by the robotic catheter system. In some embodiments, the cassette comprises a drip pod that includes the drip chamber and the clot pod, the method further comprising capturing images depicting material in the clot pod and communicating the captured images depicting material in the clot pod to a remotely located control system for displaying the material collected in the clot pod. Capturing images for one of these methods can include capturing images of the drip chamber and the clot pod by an imaging system having a single imaging device, or capturing images of the drip chamber and the clot pod by an imaging system using multiple (e.g., two) imaging devices.

### BRIEF DESCRIPTION OF THE DRAWINGS

The foregoing is a summary, and thus, necessarily limited in detail. The above-mentioned aspects, as well as other aspects, features, and advantages of the present technology are described below in connection with various embodiments, with reference made to the accompanying drawings.

FIG. 1A illustrates one embodiment of a one channel fluidics management system.

FIG. 1B illustrates one embodiment of a three channel fluidics management system.

FIG. 2 shows a schematic of a three channel fluidics system.

FIG. 3 shows another schematic of a fluidics system.

FIG. 4 shows another schematic of a fluidics system.

FIGS. 5A and 5B illustrate cross-sectional views of an exemplary rotating hemostatic valve with a gasket configured in an open position.

FIGS. 6A and 6B illustrate cross-sectional views of a rotating hemostatic valve with a gasket configured in a low pressure position.

FIGS. 7A and 7B illustrate cross-sectional views of an exemplary rotating hemostatic valve with a gasket configured in a high-pressure position.

FIGS. 8A-8C illustrate various views of an exemplary gasket for use in the hemostatic valve described herein.

FIG. 9 illustrates a perspective view of an actuation mechanism for use with the rotating hemostatic valves described herein.

FIGS. 10A-10B illustrate two different positions of the actuation mechanism of FIG. 9.

FIG. 10C illustrates a linear actuator assembly.

FIG. 11 illustrates a perspective view of an example assembly with a rotating hemostatic valve integrated with a manifold.

FIG. 12 is a flow diagram of a method for degassing a fluid management system of a robotically driven medical device.

FIG. 13 illustrates a schematic of a control system.

FIG. 14 is a schematic illustrating an example of a robotic catheter system that includes another embodiment of a fluidics system.

FIG. 15 is a schematic illustrating an example of a fluidics assembly (e.g., a disposable or recyclable fluidics assembly) that includes a cassette that couples to a pump station and couples to a saline source, a contrast source, and a vacuum source, the fluidics assembly also including a plurality of fluid communication channels to provide saline, contrast, and vacuum to mounts and catheters coupled to the mounts.

FIG. 16 is a schematic illustrating an example of a fluidics assembly that includes a cassette, a splitter, and one or more mounts, a first tubing set coupled to the cassette and splitter, and a second tubing set coupled between the splitter and the one or more mounts, the first tubing set having channels for providing saline, contrast and vacuum to the splitter, and the second tubing set having groups of channels (or subchannels) providing saline, contrast, and vacuum to each of the one or more mounts, the first and second tubing set and the splitter also providing electrical connections between the cassette and one or more connected mounts.

FIG. 17A is a schematic illustrating an example of a cassette of the fluidics assembly, the cassette including a housing that can support, all or part of a saline subsystem, a contrast subsystem, and a vacuum subsystem.

FIG. 17B is a schematic illustrating another example of a cassette of the fluidics assembly, the cassette including a housing that can support, all or part of a saline subsystem, a contrast subsystem, and a vacuum subsystem.

FIG. 18A is a schematic illustrating an example of a drip pod assembly (“drip pod”) including a drip chamber and clot pod.

FIG. 18B is a schematic illustrating another example of a drip pod including a drip chamber and clot pod.

FIG. 19 is a schematic illustrating an example of a fluidic system components in one or more mounts, which can be coupled to the second tubing set, this particular example including three mounts each having a fluidic connection for saline, contrast, and vacuum, and an electrical connection that includes one or more electrical leads, the electrical connection coupled to one or more sensors in the mounts to communicate signals from the one or more sensors to the cassette, and ultimately a station (e.g., a pump station) when the cassette is coupled to the station.

FIGS. 20A-20C are schematics illustrating another example of a contrast subsystem that can be included in a cassette.

FIGS. 20D-20E are schematics illustrating another example of a contrast subsystem that can be included in a cassette.

FIG. 21 is a schematic illustrating an example of the configuration of the fluidic system components shown in FIG. 19, where two three-way valves are configured in a “saline drip mode” to provide a saline drip to a connector that is coupled to a catheter for providing saline flow-path to a lumen of the coupled-to catheter.

FIG. 22 is a schematic illustrating an example of the configuration of the fluidic system components shown in FIG. 19, where two three-way valves are configured in a “saline drip or contrast mode” to provide a saline drip or contrast to a connector that is coupled to a catheter for providing a saline flow-path and/or a contrast flow-path to a lumen of the coupled-to catheter.

FIG. 23 is a schematic illustrating an example of the configuration of the fluidic system components shown in FIG. 19, where two three-way valves are configured in a “aspiration or backbleed mode” to provide apply atmospheric pressure or a vacuum to a connector that is coupled to a catheter for aspirating or back-bleeding a lumen of the coupled-to catheter.

FIG. 24 is a schematic illustrating an example of the configuration of the fluidic system components shown in FIG. 19, where two three-way valves are configured in a “flush with saline mode” to provide a saline flow-path with a relatively higher flow of saline (i.e., higher than when in “saline drip mode”) to a lumen of the coupled-to catheter for flushing the fluidic channels and/or the catheter with saline.

FIG. 25 is a schematic illustrating an example of the configuration of the fluidic system components shown in FIG. 19, where two three-way valves are configured in a “saline drip or contrast mode” to provide a saline drip or contrast flow to a connector that is coupled to a catheter for providing saline and/or to a lumen of the coupled-to catheter.

FIG. 26 is a schematic illustrating an example of the configuration of the fluidic system components shown in FIG. 19, where two three-way valves are configured in a “prime with saline mode” to align a saline flow-path with a relatively high volume saline flow (for example, a higher volume than a saline flow from a saline restrictor channel) for priming the mount with saline.

FIG. 27 is a schematic illustrating an example of the configuration of the fluidic system components shown in FIG. 19, where two three-way valves are configured in a “prime with contrast mode” to align a contrast flow-path for priming the mount with contrast.

FIG. 28 is a schematic illustrating examples of components of a robotic catheter system that a controller can control and examples of information that the controller can use to determine to control the components.

FIG. 29A is a flowchart illustrating a process performed by a fluidics system to monitor saline flow and switch from a first saline source to a second saline source.

FIG. 29B is a schematic illustrating an example of process for monitoring saline flow and switching from a first saline source to a second saline source based on a signal from a weight sensor, and is described in reference to the embodiment of the cassette illustrated in FIG. 17B.

FIG. 30 is a flowchart illustrating a process for performing contrast injection of the selected catheter in a robotic catheter system having multiple catheters coupled to a movable mounts with each of the movable mounts is being provided fluidic saline, contrast, and vacuum by the fluidics system.

FIG. 31 illustrates an example configuration of a fluidics system where saline is provided to a catheter, for example, before a clot is ingested.

FIG. 32 illustrates an example configuration of a fluidics system where “high” vacuum is provided to a catheter, for example, when the distal end of the catheter is positioned near a clot such that the clot can be ingested by the vacuum subsystem and captured in the clot pod.

FIG. 33 illustrates an example configuration of a fluidics system where “high” vacuum is provided to a catheter, illustrating blood flow through the catheter towards the drip pod where air bubbles are formed in the aspirated blood as a result of the applied high vacuum.

FIG. 34 illustrates an example configuration of a fluidics system where “high” vacuum is provided to a catheter and the clot has been transported back through the catheter, mount, and vacuum line and captured in a clot pod of a drip pod.

FIG. 35 illustrates an example configuration of a fluidics system where “high” vacuum is provided to a catheter and the clot has been aspirated and captured in a clot pod, and the system is configured to perform a saline flush of the clot pod and the vacuum communication channel between the mount and the clot pod.

FIG. 36 illustrates an example configuration of a fluidics system during backbleed, where “low” vacuum is provided to a catheter and to create a vacuum flow-path from the catheter to the clot pod to remove fluid containing air bubbles from the catheter and hub without decreasing the solubility of blood to the point where (additional) dissolved air comes out of the blood or saline in the catheter, hub, and/or mount.

FIG. 37 illustrates an example configuration of a fluidics system during patency check of a catheter.

FIG. 38 is an elevation view illustrating a distal side of an embodiment of a fluidic cassette (“cassette”) that is configured to be removably couplable to a pump station, the cassette including certain fluidic components for providing saline, contrast, and vacuum to one or more hubs that can be included in a robotic catheter system.

FIG. 39 is an elevation view illustrating the proximal side of an embodiment of the cassette that is shown in FIG. 38.

FIG. 40 is another elevation view of the proximal side of the embodiment of the cassette that is illustrated in FIG. 39, in this view the electrical interface is not shown to illustrate channels that run behind it.

FIG. 41 illustrates the proximal side of an embodiment of a drip chamber and clot pod that can be included in a cassette.

FIG. 42 illustrates an embodiment of the drip pod showing a main compartment (first portion) of the drip chamber and a shelf of the drip chamber which defines a second portion of the drip chamber.

FIG. 43 illustrates an embodiment of the drip chamber shown from the proximal side of the drip chamber (i.e., the side of the drip chamber that faces the pump station when the cassette is coupled to the pump station).

FIG. 44 illustrates a cutaway view of a perspective view of an upper portion of the drip chamber shown in FIG. 43.

FIG. 45 illustrates a perspective view of the proximal side of an embodiment of a clot pod.

FIG. 46A illustrates a side view of an embodiment of a drip chamber (for example, the drip chamber illustrated in FIG. 41) that can be included in a cassette, this Figure illustrating an example of the angled position of the drip chamber and its position relative to a camera positioned in a pump station that the cassette can be coupled to.

FIG. 46B illustrates a side view of an embodiment of a clot pod (for example, the clot pod illustrated in FIG. 41) that can be included in a cassette, this Figure illustrating an example of the angled position of the clot pod and its position relative to a camera positioned in a pump station that the cassette is coupled to.

FIG. 47 illustrates an example of a representation of a drip pod having a drip chamber and a clot pod in a cassette as viewed from a camera, positioned in the interface of the pump station, through a proximal surface of the cassette.

FIG. 48 illustrates an example of a pump station, and a cassette coupled to the interface of the pump station.

FIG. 49 illustrates a perspective front view of an example of a pump station interface.

FIG. 50 illustrates an elevation front view of an example of the pump station interface shown in FIG. 49.

FIG. 51 illustrates a perspective front view of an example of a pump station interface and a cassette positioned above the pump station interface.

FIG. 52 is an elevation front ghost view of an example of a cassette coupled to an embodiment of a pump station interface.

FIG. 53 is a perspective front ghost view of an example of a cassette coupled to an embodiment of a pump station interface.

FIG. 54 is an elevation front view of an example of a cassette coupled to an embodiment of a pump station interface.

FIG. 55 is a perspective view of an example of a portion of a latch assembly, on the pump station, which is used to couple a cassette to a pump station interface.

FIG. 56 illustrates another view of the latch assembly shown in FIG. 55.

FIG. 57 illustrates an example of a lower portion of a pump station interface having a recess configured to receive a correspondingly-shaped structure extending from the proximal side of a cassette to couple the cassette to the interface.

FIG. 58 illustrates a side view of an example of the lower portion of a cassette having a structure, extending from the proximal side of the cassette, which is used to help attach the cassette to the pump station interface.

FIG. 59 illustrates an example of a camera assembly that is positioned in the interface of a pump station in a location to image a drip chamber and a clot pod positioned in the cassette.

FIG. 60 illustrates an example of an air sensor positioned in the pump station interface and configured to sense air in a channel positioned in a portion of the air sensor.

FIG. 61 illustrates an example of a contrast pump actuator assembly.

FIG. 62 illustrates another view of the contrast pump actuator assembly illustrated in FIG. 61.

FIG. 63 illustrates an exploded view of the contrast pump actuator latch illustrated in FIG. 61.

### DETAILED DESCRIPTION

The foregoing is a summary, and thus, necessarily limited in detail. The above-mentioned aspects, as well as other aspects, features, and advantages of the present technology will now be described in connection with various embodiments. The inclusion of the following embodiments is not intended to limit the disclosure to these embodiments, but rather to enable any person skilled in the art to make and use the contemplated invention(s). Other embodiments may be utilized, and modifications may be made without departing from the spirit or scope of the subject matter presented herein. Aspects of the disclosure, as described and illustrated herein, can be arranged, combined, modified, and designed in a variety of different formulations, all of which are explicitly contemplated and form part of this disclosure.

Properly injecting fluids into vessels of a living human body in a precise and predictable manner can be difficult without assisted fluid management systems. Such a desired preciseness for administering fluids, combined with the danger of delivery of improper volumes of fluid or fluid containing air bubbles, has led the medical industry to train physicians with a tactile feel for fluid administration combined with a visual volume and air bubble assessment. For example, when learning to inject fluids into the brain, physicians are trained to press a syringe with a specific coordinated pressure as well as how to manually prepare and review fluids for volume and air bubbles when injecting and/or removing fluids during particular procedures.

In catheterization procedures, air emboli represent a significant, even fatal, hazard for patients. Air can be introduced during fluid injection, during catheter switching or manipulation, or any other event that creates a pressure gradient that enables air to flow into the catheter and subsequently into the vessel. Reducing the number of times that connections are broken and created in a system during a catheterization procedure may reduce the likelihood of air embolism. The fluidics management systems and methods described herein are configured to reduce the likelihood of air embolism during a catheterization procedure.

Further, in the case of ischemic stroke or other occlusive or thrombus-related conditions, every minute that goes by without treatment may result in reduced recovery for the patient. Reducing manual switching between fluid administration and removal (e.g., aspiration) catheters as well as reducing the time needed for fluid preparation during a catheterization procedure, for example using a fluid management system, may improve patient survival and recovery post the stroke event. In some embodiments, reducing manual switching between fluid administration and removal catheters by using the fluid management systems described herein may provide an advantage of reducing workload for operating staff. In some embodiments, reducing manual switching between fluid administration and removal catheters by using the fluid management systems described herein may provide an advantage of enabling a remotely controlled procedure (in which an interventionalist is not onsite near to the fluid management system and/or catheters) to be carried out in a streamlined fashion because connection changes may not be part of the procedure when using the fluid management systems described herein. In some embodiments, reducing manual switching between fluid administration and removal catheters by using the fluid management systems described herein provides an advantage of improved safety and reliability (e.g., procedure step consistency). In addition, by using a consistent fluid management system, the risk of air embolization is reduced.

Disclosed herein are systems and methods for managing fluidics systems that administer and remove fluids during medical procedures. The fluidics systems described herein can be used with robotic catheter systems. The fluidics systems can be used with other device and methods as well. The fluidics systems may be coupled to robotically driven interventional devices, manually driven interventional devices, or any combination thereof. In particular, the systems and methods may be configured to control fluid administering equipment to ensure proper diagnostics and/or treatment is provided.

The systems and methods described herein may include a programmable and/or automated fluid injection and removal system that may assist a physician (e.g., surgeon, interventionalist, and the like) to perform procedures when fluidics are involved. For example, the devices, systems, and methods for operating fluid management systems described herein may automate fluid injected using programmable pumps, vacuums, catheter hubs, and the like, to allow consistent, precise, and timely injections. In some embodiments, the fluid lines are not swapped or disconnected during a procedure, but are instead configured once before a procedure and left intact throughout the procedure so as to avoid errors at connection points (e.g., valving errors), errors in fluid selection, and/or air bubble introduction issues that arise because of air introduced when switching between fluids.

In operation, the fluid management systems described herein may include multiple fluidics systems in which each system is for a separate fluid source and or fluid collection container. The multiple fluidics systems may be configured to flow from the same fluid management system and couple to interventional devices or medical tools. For example, each fluidics system may be configured to connect to each catheter hub (manual catheter hub or robotically driven hub) associated with the fluid management system. Each catheter hub (“hub”) may connect to at least one interventional device, for example a catheter. The hub, or a control interface removed from the hub, may include controls to control fluid administration steps and/or catheter manipulation steps.

In some embodiments, the devices, systems, and methods described herein may be configured to provide an advantage of reducing the time and effort used to degas fluidics systems. For example, maintaining fully filled fluid lines and fluid tubing connections during procedures (i.e., avoiding switching of fluidics components) may ensure that a degassing procedure can be performed a single time for each fluid before the procedure. The methods described herein may include configuration methods, degassing methods, methods of treatment, fluid injection and/or fluid removal methods, and the like.

In some embodiments, the devices, systems, and methods described herein may be configured to reduce a number of sterile packages that have to be opened for a particular procedure. For example, because additional catheters, fluid lines, hubs, and/or other fluidly connected components are configured to connect to the system before the procedure and remain connected, the components may be packaged together or at least multiple packages opened and components assembled at one time before the procedure and there may be no need to open additional packages or install additional components during the procedure. For example, in certain embodiments, one or more catheters, hubs, and fluid lines may be provided in a single sterile package. In some embodiments, one or more catheters, hubs, and fluid lines may be provided in a single sterile package with a splitter and/or a cassette. In other embodiments, any of the foregoing components may be packaged in separate sterile packages.

In some embodiments, the devices, systems, and methods described herein may be configured to reduce time and/or steps used to flush fluid lines because such steps may be automated and performed in an automatic fashion when requested by the fluid management system.

In some embodiments, the devices, systems, and methods described herein may be configured to display fluidics management steps on a user interface to streamline configuration of the fluidics systems. For example, specific user interfaces may be configured for specific procedures. Each user interface may present instructions, information, or other data to a procedure staff while the fluidics management system is automatically executing next steps of the procedure with respect to fluidics management.

### Systems and Devices

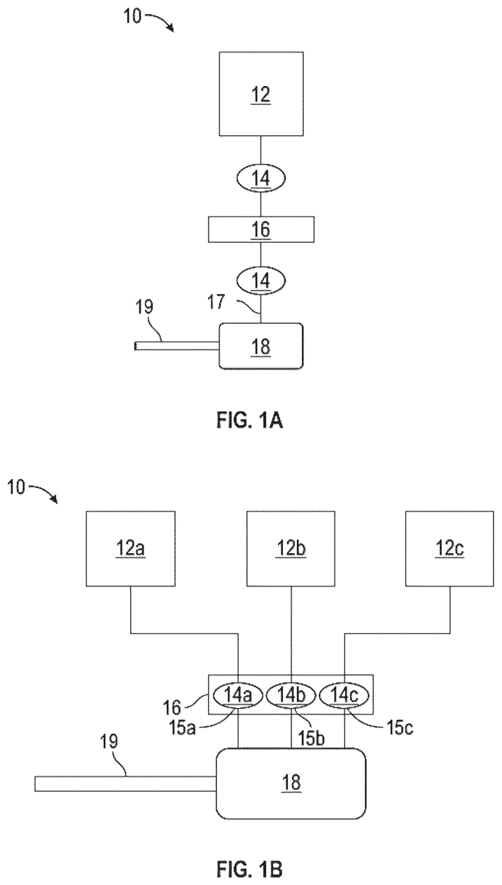

FIG. 1A illustrates one embodiment of one channel of a multi-channel fluidics management system 10. The fluidics management system 10 may be configured as an automated system to manage the delivery of fluids to or aspirate material from a patient via one or more interventional devices, for example catheters. As shown, the system 10 may manage fluid delivery to a patient during a medical procedure. The fluidics management system 10 includes at least one fluid source and/or sink 12 coupled to a valve 14, which is coupled to a manifold 16. The manifold 16 is either remote (e.g., on a support table or tower outside the sterile field) or coupled to a catheter hub 18, which is coupled to at least one source and/or sink line 17. The source and/or sink line 17 is coupled through the hub to at least one catheter 19.

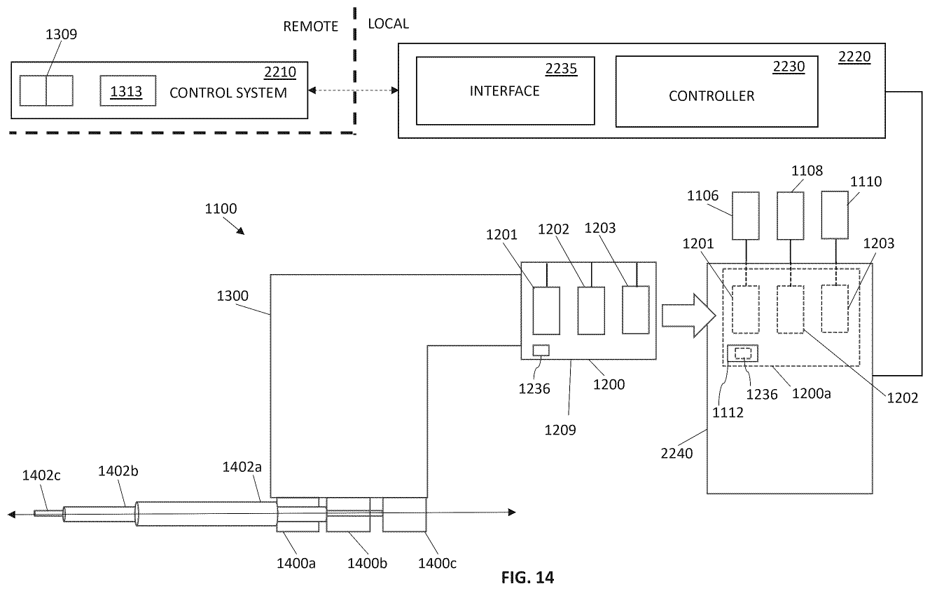

Certain embodiments of hub assemblies described herein, such as hub assembly (“hub”) 18, include a housing for coupling an interventional device thereto, components (e.g., rollers) for directly coupling to and moving along a drive table, and magnet(s) for magnetically coupling to a hub adapter across a sterile barrier. A hub (or hub assembly) can refer to a single assembly with a housing, or a hub (or hub assembly) can generally refer to an apparatus having two (or more) subassemblies (e.g., a first subassembly and a second subassembly). In some embodiments of a hub assembly having two subassemblies, a hub can refer to a first subassembly that can be configured to couple to and house an interventional device, and that may be removably attachable to a second subassembly (or mount) configured to magnetically couple to a hub adapter across a sterile barrier and move along a drive table. Such a hub and mount may together form a hub assembly. Such hub assemblies may allow for a hub (first subassembly) to be removed from a mount (second subassembly) which can be advantageous, for example, so that a different hub can be coupled to the same mount or so that the hub may be used separately from the mount (e.g., for a manual procedure). Certain components of advantageous example configurations of a mount are shown and described in reference to FIGS. 14-37. An arrangement of a hub assembly having a hub that is releasably couplable to mount can allow for replacement of a hub with a different hub having a different interventional device coupled thereto without breaking a magnetic connection with a hub adapter. For example, such an arrangement may allow for a hub coupled to an access catheter to be removed from a mount and replaced with a hub coupled to a procedure catheter without breaking a magnetic connection between active and passive magnetic sides of the coupling of the hub adapter and hub assembly (e.g., between the hub adapter and the mount). In some embodiments, the mount may be a magnetically driven member, an axially driven member, a puck, a slider, a shuttle, or a stage. The fluidics systems described herein can relate to various embodiments of systems that include a hub, or a hub and a mount, regardless of whether they are described in reference to a hub, or a hub and mount, unless explicitly indicated or indicated by context.

The fluidics management system 10 can provide multiple fluids (e.g., saline, contrast) and vacuum to one or more hubs. An elongated device can be attached to each of the one or more hubs for performing a medical procedure. For example, a catheter can be coupled to each of the one or more hubs that receive fluids and vacuum from the fluidics management system 10, and a guidewire can be coupled to a hub that does not receive fluids or vacuum. The one or more hubs can be controlled by a controller of the robotic catheter system to move along a drive table to correspondingly move the attached elongated device towards or into a patient, or to move the attached elongated device away from or out of a patient. In some preferred embodiments, the one or more hubs are moved in a longitudinal direction (e.g., along a line or straight path) during use in medical procedures.

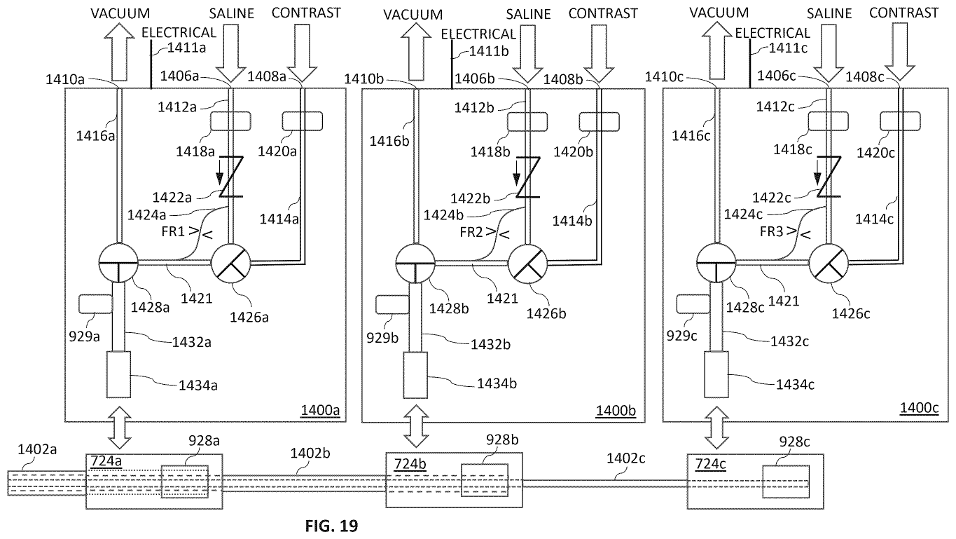

As indicated above, in some embodiments, a hub is a two-part hub. A two-part hub can include a first part (which may be referred to as a “hub” or “first subassembly”) and a second part (which may be referred to as a “mount” or “second subassembly”). An elongated device (e.g., a catheter, a guidewire, etc.) can be coupled to the hub (first subassembly). The hub can be, and typically is, mechanically coupled to the mount (second subassembly) to form the hub assembly when in use. In some preferred embodiments, the first and second subassemblies can be decoupled, if necessary, for example, during a medical procedure where the medical practitioner at the local site (with the patient) decouples the hub from the mount to perform a particular action. In embodiments where a hub includes a first and second subassembly, different components can be included in each of the first and second subassemblies. In some embodiments, the first subassembly can include a hemostasis valve controllable by a controller of the robotic catheter system to open or close during a medical process. For example, the hemostasis valve can be controlled to close during a contrast injection process with a first catheter to control the flow of contrast through the catheter lumen to the distal tip of the first catheter and preventing the injected contrast from flowing in the opposite direction along the surface of another elongated device positioned at least partially in the lumen of the first catheter. The first subassembly can include rotational mechanisms for rotating the elongated device coupled to the first subassembly. The rotational mechanisms can be driven by actuators in the second assembly which couple to the rotational mechanisms when the first subassembly is coupled to the second subassembly. In various embodiments, the second subassembly can include fluidics components (e.g., air sensors, fluid connectors, air bubble filters, pressure sensors, control valves, channels, saline restricted-flow channels, and the like) that are used to provide fluids and vacuum to a catheter attached to the first subassembly. An example of a two-part hub is illustrated in FIG. 19, which illustrates an embodiment of a system that includes three hub assemblies. In this embodiment, three second subassemblies (mounts) 1400a-c provide fluids and vacuum to connectors 1434a-c, which can be connected to first subassemblies 724a-c which are coupled to catheters 1402a-c, respectively, to provide saline, contrast, and vacuum to the catheters 1402a-c. Each of the first subassemblies 724a-c includes a hemostasis valve 928a-c. In this embodiment, each of the first subassemblies 724a-c is mechanically, and fluidically, releasably couplable to the respective second subassemblies 1400a-c. Whether a hub includes a “single” hub assembly or includes a first subassembly and a second subassembly, all or nearly all of the fluidic operations are the same when the first subassembly and a second subassembly are coupled together. A two-part hub may be referred to herein simply as a hub for ease of reference. In some instances, for a hub having a first subassembly and a second subassembly, the first subassembly may be referred to herein as a “hub” and the second subassembly may be referred to as a “mount” to differentiate the two subassemblies.

In some embodiments, the fluid source and/or sink 12 includes both a reservoir of fluid volume and a means of propelling such fluid to another component of the system 10 or a means of retrieving fluid back to the source. Example propelling means may include one or more propellers, impellers, and/or pumps to circulate and/or retrieve fluid throughout system 10. In some embodiments, the propelling means can be used to control the volume, flow rate, and/or pressure. In certain embodiments, the propelling means can be activated to propel fluid to another component of the system or retrieve fluid from the system or deactivated to stop the movement of fluid.

In some embodiments, the fluidics management channel is substantially duplicated for each catheter configured for use in a particular medical procedure. Different channels may differ in sensors, pumps, and/or valves employed based on the interventional device that is connected to each fluidics channel. For example, a fluidics system for a procedure catheter (e.g., for aspiration) may include an inline vacuum pump and filter. Further for example, a fluidics system for a guide, access, or insert catheter may include an inline drip rate sensor, air bubble sensor, pressure sensor, and/or air bubble filter.

The source and/or sink 12 represents either a fluid source or a fluid sink (e.g., waste canister). For example, a fluid source may include a container adapted to house a fluid (e.g., saline, contrast, pharmaceuticals, blood, plasma, or other fluid) for use with the fluidics management system 10. The container may be configured to release fluid into a fluid delivery line (e.g., fluid delivery tube) using active means (e.g., pumps, vacuums, etc.) or passive (e.g., gravity). The fluid sink may include a container adapted to receive fluids (e.g., aspirate, thrombus, particulate, saline, contrast, pharmaceuticals, blood, or other fluid or combination thereof) from the patient and/or from other fluidics infrastructure within the fluidics management system 10.

The valve 14 represents one or more valves that are coupled to the source and/or sink 12 at a first side of the valve 14 and coupled to the manifold 16 at a second side of the valve 14. The manifold 16 is configured to connect each valve 14 to a particular hub 18. In some embodiments, the valve 14 may instead couple directly to the hub 18 to avoid the use of a separate manifold 16. In some embodiments, the manifold 16 may be integrated into the hub. In some embodiments, a second valve 14 may connect the manifold 16 to the hub 18. For example, the second valve 14 can be coupled to the manifold 16 at a first side and coupled to the hub 18 at the second side.

The hub 18 is configured to releasably or non-releasably couple to an interventional device (i.e., a catheter, guidewire, or other medical device). For example, a catheter 19 has a proximal end attached to a hub 18. In some embodiments, the hub 18 is moveable along a path along the surface of a robotic drive table to advance or retract the catheter 19 (or other medical and/or interventional device). Each hub 18 may also contain mechanisms to rotate or deflect the catheter 19 or guidewire as desired. The hub 18 may be connected to fluid delivery tubes (e.g., source/sink line 17) to provide fluid release or fluid capture. Each hub 18 may be in electrical communication with an electronic control system, either via hard wired connection, RF wireless connection, or a combination of both. Additional details of the hubs, drive table and related systems are found in U.S. patent application Ser. No. 17/816,669, entitled Method of Supra-Aortic Access for a Neurovascular Procedure, filed Aug. 1, 2022, which is hereby expressly incorporated by reference in its entirety herein.

Any of the hubs disclosed herein may further comprise one or more fluid injection ports and/or a wireless RF transceiver for communications and/or power transfer. In some embodiments, the hub 18 may also comprise a wired electrical communications port and a power port.

In some embodiments, the hub 18 or line 17 leading to the hub 18 may include a visual indicator, for indicating the presence of an aspirated clot. The visual indicator may comprise a clot chamber having a transparent window. A filter may be provided in the clot chamber. Additional details of the clot capture filter and related features may be found in U.S. Provisional Patent Application Ser. No. 63/256,743, entitled Device for Clot Retrieval, filed Oct. 18, 2021, which is hereby expressly incorporated by reference in its entirety herein.

Any of the hubs or interventional devices disclosed herein may further comprise a sensor for detecting a parameter of interest such as a location or orientation of a distal tip or a status of the distal tip of an interventional device. The status of the distal tip may include, but not be limited to detection of an interaction between a vessel wall and the distal tip, detection of an interaction between a vessel wall and a clot, or detection of an unobstructed distal tip. The sensor, in some instances, may be positioned on a flexible body of an interventional device. The sensor may comprise a pressure sensor to capture arterial blood pressure waveform at the distal end of the catheter, or an optical sensor to determine captured clot or air bubbles. In some embodiments, the sensor may comprise one or more of: a force sensor, a positioning sensor, a temperature sensor, a torque sensor, a strain sensor, and/or an oxygen sensor. In some embodiments, the sensor may comprise a Fiber Bragg grating sensor. For example, a Fiber Bragg grating sensor (e.g., an optical fiber) may detect strain locally that can facilitate the detection and/or determination of force being applied.

FIG. 1B illustrates a schematic view of multi-channel fluidics management system 10 having a first source 12a, a second source 12b, and a sink 12c. The first source 12a is coupled to a valve 14a. The second source 12b is coupled to a valve 14b. The sink 12c is coupled to a valve 14c. The valves 14a, 14b, 14c, (hereinafter “valves 14a-14c”) are part of a valve manifold 16. The valve manifold 16 is coupled to a hub 18. In certain embodiments, the valve manifold 16 is part of or directly connected to the hub 18. In other embodiments, the valve manifold 16 is positioned remotely from the hub 18 and is connected to the hub 18 via one or more fluid lines. In other embodiments, the valve manifold is part of or directly connected to hemostatic valve. In other embodiments, the valve manifold 16 is positioned remotely from the hemostatic valve and is connected to the hemostatic valve via one or more fluid lines. The valves 14a-14c can be opened and closed to selectively place the first fluid source 12a, the second fluid source 12b, and the sink 12c in communication with a lumen of the catheter 19. For example, the valve manifold 16 can include fluid ports (e.g., a first port 15a associated with valve 14a, a second fluid port 15b associated with valve 14b, and a third port 15c associated with valve 14c) that can be selectively placed in communication with the lumen of the catheter 19 or blocked from communication with the lumen of the catheter 19. For example, in some embodiments, one of the first port, second port, and third port can be placed into communication with the lumen of the catheter 19 while the other two ports are blocked from communication with the catheter 19.

In certain embodiments, the first source 12a can be a source of heparinized saline. The source 12b can be a source of contrast solution. In certain embodiments, one or more of the source 12a, the source 12b, and the source 12c can couple to a plurality of manifolds 16, each coupled to a unique interventional device 18. The valve manifold 16 as shown herein may be utilized in any of the systems described herein.

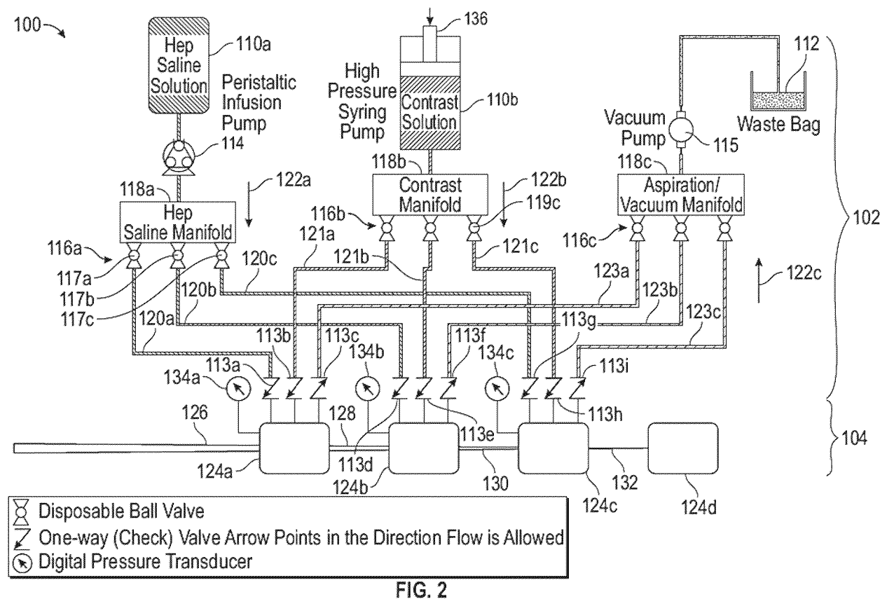

FIGS. 2-4 and 13-27 illustrate schematic views of various examples of fluidics systems, features, and operations of fluidic system, for providing saline, contrast, and/or vacuum to one or more interventional devices. Features and structure illustrated and described with respect to one of these fluidic systems are contemplated to be used in the other disclosed fluidic systems, and generally in other fluidic systems. FIG. 2 illustrates a schematic view of a three channel fluidics system 100 for use with a fluidics management system 10 including a stack of four concentric interventional devices. The fluidics system 100 shown here includes a fluid management portion 102 and an interventional portion 104. In some embodiments, the interventional portion 104 may comprise a concentric catheter and guidewire stack configured for manual manipulation by the physician. In some embodiments, the interventional portion 104 may comprise a concentric catheter and guidewire stack configured for manipulation by a robotic drive system. In some embodiments, the interventional portion 104 includes a combination of both robotically driven medical devices and manually manipulated medical devices.

The components of fluid management portion 102 may be located outside of the sterile field or within the sterile field. In some embodiments, the fluid management portion 102 is located outside of the sterile field, but is coupled to the interventional portion 104, which is located within the sterile field, by flexible tubing and flexible electrical conductors.

The fluid management portion 102 may include at least two or three or more channels (e.g., parallel channels) of the type shown in FIG. 1A, each for a separate fluid source or fluid sink. In the illustrated embodiment, the fluid management portion 102 includes three channels that each are in communication with each of three catheters via corresponding catheter hubs. Two channels provide for the delivery of two separate fluids to each of the catheters, each at a controllable pressure, volume and delivery rate. The third channel provides for aspiration from each catheter into a sink.

Each of the two or more fluid channels may be primed by completely degassing and filling with a respective fluid in order to be ready for transport into the catheter and into the bodily lumen. In some embodiments, fluid lines, catheters, and/or catheter lumens can be simultaneously flushed and primed with a fluid (e.g., saline).

In some embodiments, the fluidics systems 100, 200 may be configured to backfill each sink connection to each catheter with fluid (e.g., saline) at procedure initialization and/or between fluidics step. This may provide a backfilled column of saline downstream of the sink connection, for example, to ensure that contrast injections flow to a distal tip of a particular catheter rather than to the sink. In some embodiments, the fluidics systems 100, 200 may be configured to provide a backfilled column of saline upstream of a saline valve at the hub, for example, to ensure that contrast injections flow to the distal tip of a particular catheter or the sink rather than through the saline valve.

As shown in FIG. 2, the fluid management portion 102 of the fluidics system 100 includes a first source 110a, a second source 110b, and a sink 112. The first source 110a and the second source 110b may each be configured to hold and distribute at least one fluid (e.g., saline, contrast, pharmaceuticals, blood, or other fluid, or combinations thereof). The sink 112 may be configured to receive waste fluid and/or waste product from a selected aspiration line leading to a corresponding catheter. Although two fluid sources and one fluid sink are shown, any number of fluid sources and/or fluid sinks is possible (e.g., one fluid source and one fluid sink, two fluid sources without a fluid sink, more than two fluid sources, etc.) corresponding to the fluid delivery and/or aspiration needs of a particular procedure.

A number of valves (and/or valve arrays) are provided to stop and start flow of each respective fluid to or through one or more fluid lines, and/or hubs within the fluid management portion 102 and/or interventional portion 104. In the illustrated implementation, a first valve array 116a (e.g., with three valves) is carried by a first manifold 118a, a second valve array 116b (e.g., with three valves) is carried by a second manifold 118b, and a third valve array 116c (e.g., with three valves) is carried by a third manifold 118c. Although valve arrays with three valves are shown, any number of valves are possible and may correspond to the number of catheters and/or fluid sources being used in the procedure or a subset of the interventional devices being used in the procedure. For example, in some situations, each valve array may include at least one valve, two valves, three valves, or four or more valves.

In some embodiments, each valve in a valve array (e.g., first valve array 116a) may be configured to independently control and/or adjust fluid resistance, flow rate, and/or pressure of fluid flowing through the valve and corresponding tubing. In some embodiments, each valve in a valve array can be independently and/or simultaneously adjusted for a respective catheter and/or for more than one catheter.

In the illustrated implementation, the fluidics channel is duplicated for each catheter and will therefore be described only in connection with the first source 110a below. A first outflow valve 117a is in communication with a first catheter 126 by a unique first fluid source line 120a. A second outflow valve 117b is in communication with a second catheter 128 by a unique second fluid source line 120b. A third outflow valve 117c is in communication with a third catheter 130 by a unique third fluid source line 120c. The fluid source lines 120a, 120b, 120c may be hereinafter referred to as the “fluid source lines 120a-120c.” Each of the outflow valves 117a, 117b, 117c (hereinafter “outflow valves 117a-117c”) is preferably electronically actuated in response to signals from the control system, between a fully closed, fully open, or partially open positions. Any of a variety of valve mechanisms maybe utilized, such as a ball valve driven by a stepper motor, solenoid, a stopcock valve (e.g., a rotating stopcock valve), a rotary valve, or other drive mechanism known in the art. The drive mechanisms may provide for automated control and sequencing of the valves. For example, valve actuation may be achieved using stepper motors with in-built encoding to provide consistent switching and sequencing. The drive mechanisms may be controlled using motor controllers of a user control interface (for example, or a computer system). The control system may include modules that read values from sensors (e.g., flow, bubble, pressure, etc.) and display the values to control the behavior of the fluid system.

In some embodiments, a stopcock valve mechanism (e.g., a rotating stopcock valve) may be used in the manifolds described herein. For example, one or more stopcock valves may be placed adjacent to (or integrated into) a hub to avoid management of a column of fluid in particular tubing. Such tubing may be sterile disposable tubing that may offer a one-time use. Placing a manifold with stop cock valves near or integrated into the hub has the advantage of simplicity without the need to manage a column of fluid in the tubing. Having the manifold and stopcock valves away from the hub(s) may allow the manifold and the stopcock valves to both be used outside of a sterile field in conjunction with non-sterile equipment. Such a configuration may provide an advantage of preserving sterility of the components in the sterile field.

In some embodiments, the fluidics control system may further include a drive mechanism configured to adjust the sealing strength of the hemostatic valve in response to a signal from the control system, for example, from a processor of the control system. The control system (e.g., the processor) may be configured to increase the sealing strength of the hemostatic valve in response to the manipulation of the contrast control to introduce contrast into the catheter. The control system (e.g., the processor) may additionally be configured to decrease the sealing strength of the hemostatic valve in response to the manipulation of the contrast control to stop introducing contrast into the catheter. In some embodiments, the control system (e.g., the processor) may be configured to decrease the sealing strength of the hemostatic valve in response to a signal received to drive a catheter or guidewire through the hemostatic valve. Such a feature may provide the advantage of reducing friction between the hemostatic valve and a moving catheter shaft, for example.

In operation, all three outflow valves 117a-117c maybe in an open configuration to flow saline through each of the three catheters. Forward flow (in the direction of arrow 122a) of saline may be driven by a pump 114 such as an electronically controlled peristaltic infusion pump or a rotary piston pump. Alternatively, any one of the valves may be open with the other two closed depending upon the desired performance. Alternatively or additionally, other sources of volume and/or pressure (for example, pump 114) can be deactivated or disconnected to prevent flow.

In the concentric catheter stack illustrated in FIG. 2, the first catheter 126 may be a ‘large bore’ access catheter having a diameter of at least about 0.075 or at least about 0.080 inches in diameter. The second catheter 128 may be an aspiration catheter having a diameter within the range of from about 0.060 to about 0.075 inches. The third catheter 130 may be a steerable catheter with a deflectable distal tip, having a diameter within the range of from about 0.025 to about 0.050 inches. The guidewire 132 may have a diameter within the range of from about 0.014 to about 0.020 inches. In one example, the first catheter may have a diameter of about 0.088 inches, the second catheter about 0.071 inches, the third catheter about 0.035 inches, and the guidewire may have a diameter of about 0.018 inches.

The available lumen in the first catheter 126 is the difference between the inner diameter (ID) of first catheter 126 and the outer diameter (OD) of second catheter 128. That may be different than the available lumen in the second catheter 128 (which may be the difference between the ID of second catheter 128 and the OD of third catheter 130), which may be different than the available lumen of the third catheter 130 (which may be the ID of the third catheter 130 or the difference between the ID of the third catheter 130 and the OD of the guidewire 132). In order to produce the same delivered infusion flow rate through each of the catheters, the control system may be configured to adjust the pump 114 and/or each of the outflow valves 117a-117c to compensate for differences in the effective cross sections of each respective flow-path in order to achieve the same delivered flow rate through each catheter. “Flow-path” as used herein is a broad term that can refer to a path for communication of fluid through one or more channels, lines, tubes, lumens, and other structures (e.g., portions of a valve, pump that communicates fluid) that can communicate blood, saline, contrast, vacuum, or other fluids and gasses. “Flow-path” can also refer to the structures themselves (i.e., one or more channels, lines, tubes, lumens, portions of a valve, pump that communicates fluid, and other structures that communicate fluid and can be considered synonymous and interchangeably with “channel” unless based on context or as explicitly stated. In an example, a saline subsystem can be described as having a saline flow-path that receives saline from a saline source and provides saline to downstream saline communication channels and to mounts/hubs, a contrast subsystem (e.g., for priming the contrast subsystem and downstream communication channels), and a vacuum subsystem (for priming a portion of the saline subsystem), referring to a plurality of branches of a saline flow-path formed by channels, tubes, etc. In an example, a contrast subsystem can be described as having a contrast flow-path that receives contrast from a contrast source and provides contrast to downstream contrast communication channels and to mounts/hubs, and to a vacuum subsystem (for priming the contrast subsystem), referring to one or more branches of a contrast subsystem formed by channels, tubes, etc. In another example, a vacuum subsystem can be described as having a vacuum flow-path that has vacuum from a vacuum source and provides vacuum to the saline and contrast subsystem (for priming the saline and contrast subsystem) and provides vacuum communication channels and to mounts/hubs, referring to one or more branches of a vacuum subsystem formed by channels, tubes, etc.

In one implementation of the invention, the catheters may be assembled into the concentric stack orientation illustrated in FIG. 2 prior to flushing the catheters to remove air by displacing it with a fluid such as saline. This is preferably accomplished in each fluid lumen, such as, for example, the annular lumen between the first catheter 126 and second catheter 128 and in between each of the additional concentric interventional devices in the stack orientation. Infusing saline under pressure may displace substantially all of the air but some small bubbles may remain, adhering, for example, to the inside wall of the first catheter 126, the outside wall of second catheter 128, or both.

While saline is being introduced under pressure into the proximal end of the annular lumen between two interventional devices (for example, the annular lumen between the first catheter 126 and the second catheter 128), the inner catheter may be moved with respect to the outer catheter (for example, the second catheter 128 may be moved with respect to the outer catheter), to disrupt the holding forces between the microbubbles and adjacent wall and allow the bubbles to be carried downstream and out through the distal opening of the lumen. The catheters may be moved axially, rotationally or both with respect to each other. In one implementation, a first catheter is moved reciprocally with respect to the adjacent catheter or guidewire, such as axially through a range of from about 0.5 inches to about 10 inches, or from about 1 inch to about 5 inches at a reciprocation frequency of no more than about 5 cycles per second or two cycles per second or less.

Reciprocation of adjacent catheters to disrupt microbubbles may be accomplished manually by grasping the corresponding catheter hubs and manually moving the catheters axially or rotationally with respect to each other while delivering pressurized saline. Alternatively, such as in a robotically driven system, a processor maybe configured to robotically drive at least one hub of two adjacent catheters (for example, at least one of first catheter hub 124a and second catheter hub 124b) to achieve relative movement between the adjacent catheters thereby disrupting and expelling microbubbles, such as in response to user activation of a flush control.

The second source 110b is in fluid communication with the second manifold 118b, allowing fluid to flow as shown by arrow 122b to any number of valves (e.g., three) within the second valve array 116b. Forward flow (in the direction of arrow 122b) of contrast may be driven by a pump 136 such as a syringe pump, high pressure positive displacement pump, contrast injection pump, etc. Any one of the valves of the second valve array 116b may be open with the other two closed depending upon the desired performance. Alternatively or additionally, other sources of volume and/or pressure (for example, pump 136) can be deactivated or disconnected to prevent flow. A proximal opening of each fluid source line 121a, 121b, 121c (hereinafter “fluid source lines 121a-121c”) may be coupled to a respective output port on the corresponding valve within the second valve array 116b. A distal opening of each of the fluid source lines 121a-121c may be coupled to each of the respective catheter hubs 124a, 124b, 124c (hereinafter “catheter hubs 124a-124c”), and thus to the corresponding first catheter 126, second catheter 128, and/or third catheter 130. The respective first catheter 126, second catheter 128, third catheter 130, and/or guidewire 132 may be guided into a patient (not shown). Additional hubs and/or catheters may be added to the fluidics system 100 and corresponding fluidics management system components (e.g., system 10) may be added to the fluidics system 100. In other embodiments, the fluidics system 100 may include less hubs and/or catheters, for example two hubs and/or catheters.

The sink 112 is coupled to a third manifold 118c to receive fluid from aspiration lines 123a, 123b, 123c (hereinafter “aspiration lines 123a-123c”) in the direction shown by arrow 122c. The aspiration lines are configured to receive fluid and embolic material from one or two or all three respective catheters 126, 128 and 130 depending upon input from the physician into the control system. Once the physician has determined which catheter(s) will be placed into aspiration mode, and actuated the corresponding aspiration control(s) the corresponding valve(s) within the third valve array 116c may be opened to allow the fluid to flow through the corresponding catheter and into the sink 112, in response to the control system activating an aspiration pump 115. Any one of the valves of the third valve array 116c may be open with the other two closed depending upon the desired performance. Alternatively or additionally, other sources of volume and/or pressure (for example, aspiration pump 115) can be deactivated or disconnected to prevent flow.

In an example embodiment, the fluidics system 100 represents an aspiration configuration in which the first source 110a contains heparinized saline and the second source 110b contains contrast solution. The sink 112 in this example may contain waste blood/saline/embolic material that has been aspirated from a patient (not shown). Other additional sources and/or sinks may be used in combination with respective fluids.

Similarly, the contrast solution contained by the second source 110b may flow in the direction of arrow 122b and may flow into the second manifold 118b. In a given procedure, the physician may determine to inject contrast through any of the three catheters, and typically through the most distal catheter at a given injection time. In response to an inject contrast command, the control system will open the valve corresponding to the selected catheter and typically maintain the other two valves closed. In some embodiments, the physician may inject contrast concurrently into two or more catheters. In some embodiments, for example, while driving catheters or guidewires, contrast or aspiration may be applied concurrently.

In some embodiments, each valve (or valve array) can be housed inside or carried by a respective catheter hub 124a-124c. In some embodiments, each valve (or valve array) may be housed adjacent to or remote from a respective hub. In such examples, additional fluid lines (e.g., fluid source lines 120a-120c, fluid source lines 121a-121c, and aspiration lines 123a-123c) may be added between each manifold and a corresponding valve. The fluid source lines 120a-120c, 121a-121c, and the aspiration lines 123a-123c may be tubes. In some embodiments, any of the fluid source lines 120a-120c, 121a-121c, and the aspiration lines 123a-123c may be removably coupled to their respective hubs. Alternatively, any of the fluid source lines 120a-120c, 121a-121c, and aspiration lines 123a-123c may be inseparably connected to the hubs and removably coupled to other components of the fluid management portion 102, such as the valve arrays 116a, 116b, 116c (hereinafter “valve arrays 116a-116c”) or manifolds 118a, 118b, 118c (hereinafter “manifolds 118a-118c”).

In some embodiments, the fluidics system 100 may also include any number of pressure sensors, volume sensors, flow rate sensors, tubing sets, connectors, bubble sensors/detectors as will be discussed. In the illustrated implementation a pressure transducer 134a is in pressure sensing communication with the first catheter 126 by way of the first catheter hub 124a. Additional pressure transducers 134b, 134c may be placed in communication with their corresponding catheters as illustrated.

The control system may be configured to automatically adjust the various manifold valves, pumps and hemostatic valves (discussed below) in response to commands input by the physician. For example, the physician might input a command to infuse contrast through the third catheter 130. The control system may cause a series of responsive events to automatically occur. At least the saline valve (e.g., the third outflow valve 117c) would close. Outflow valves 117a, 117b may be closed or may remain open to provide positive pressure through the first and second catheters, to prevent backflow of contrast.

A control signal will be sent to a hemostasis valve in each of the first catheter hub 124a and second catheter hub 124b, to clamp down from a low pressure sliding fit to a high pressure clamp around the second catheter 128 and third catheter 130 respectively. This will prevent contrast from escaping proximally through the first catheter 126 and second catheter 128. A control signal will additionally be sent to a valve 119c to place the third catheter 130 in fluid communication with the second source 110b containing contrast solution.

If the space between the OD of the guidewire 132 and the ID of the third catheter 130 is insufficient to allow a desired contrast infusion rate, a further signal will be sent from the control system to the drive system controlling a guidewire hub 124d, to proximally retract the guidewire 132 from the third catheter 130 a distance sufficient to allow the flow of contrast through the third catheter 130. An additional control signal may be sent to a hemostasis valve carried by the third catheter hub 124c to clamp in a high pressure mode around a distal portion of the guidewire 132 or to clamp into a completely closed configuration if the guidewire 132 was fully retracted. A further control signal may be sent to an electronically activated high pressure pump 136 such as a syringe pump, high pressure positive displacement pump, contrast injection pump, etc., to deliver contrast solution through the third catheter 130.

If the physician initiates a command to perform aspiration through, for example, the first catheter 126, the control system may automatically transmit another series of control signals to execute the command. Signals will be sent to each of the hemostasis valves to move them from the high pressure configuration to the low pressure configuration in which there is less friction generated against a shaft of the catheter or guidewire. Such a configuration may permit relative movement of the various devices and proximal retraction of the second catheter 128 and third catheter 130 from first catheter 126 while still inhibiting proximal blood loss through the hemostasis valves. Signals will be sent to the drive system to proximally retract each of the catheter hubs 124a-124c and the guidewire hub 124d. Check valve 113c will be opened to place the first catheter 126 into fluid communication with the sink 112. A signal will be sent to actuate the aspiration pump 115, thereby aspirating blood and thrombus into the sink 112. In some embodiments, when performing aspiration of the first catheter 126, for example, communication between the first catheter 126 and the first source 110a and the second source 110b may be obstructed. For example, the corresponding valves of the valve arrays 116a, 116b may be closed to obstruct the manifolds 118a, 118b. Alternatively, the sources of volume and/or pressure (for example, pump 114 and pump 136) may be deactivated or disconnected.

All of the fluid lines between the first source 110a and second source 110b and each of the catheters, and all of the fluid lines between sink 112 and each of the catheters are preferably completely flushed free of any bubbles and filled with a fluid such as saline during system preparation before the procedure. This allows seamless transition between infusion, aspiration and manipulation of the catheters and guidewire without the need to disconnect and reconnect any fluid lines between the sources, sink and catheters, eliminating the risk of introducing air emboli during such exchanges.

It may also be desirable to enable confirmation of the absence of bubbles in any of the fluid lines. This may be accomplished placing bubble sensors in bubble sensing proximity to each of the fluid lines, such as in or upstream of each of the hubs, or at the manifolds. This may be particularly desirable in a telemedicine application, where the physician is at a remote workstation, and out of direct line of sight from the patient.

This may be accomplished using a non-contact ultrasonic flow sensor that measures the intensity and doppler shift of the reflected ultrasound through the sidewall of fluid tubing to detect bubbles and measure fluid flow rate or fluid level. An ultrasonic or optical sensor may be positioned adjacent an incoming fluid flow-path within the hub, or in a supply line leading to the hub.

For example, to detect the presence of air bubbles in the infusion line (that is formed of ultrasonically or optically transmissive material) the sensor may include a signal source on a first side of the flow-path and a receiver on a second side of the flow-path to measure transmission through the liquid passing through the tube to detect bubbles. Alternatively, a reflected ultrasound signal may be detected from the same side of the flow-path as the source due to the relatively high echogenicity of bubbles.

Alternatively, an optical sensor may be provided to detect changes in optical transmission or reflection due to the presence of bubbles, or to transmit a visual signal to a display at the remote workstation where the physician can visually observe the presence of a bubble moving through the tubing. In a system having a bubble detector, the control system can be configured to automatically shut down all fluid flow in response to the detection of a bubble to give personnel an opportunity to plan next steps.

In one implementation, a bubble removal system is automatically activated upon detection of in line bubbles. A processor may be configured to activate a valve positioned in the flow-path downstream of the bubble detector, upon the detection of bubbles. The valve diverts a column of fluid containing the detected bubble out of the flow-path leading to the patient and instead into a bypass flow-path or reservoir. Once bubbles are no longer detected in the flow-path and after the volume of fluid in the flow-path between the detector and the valve has passed through the valve, the valve may be activated to reconnect the source of fluid with the patient through the flow-path. In some embodiments, the flow-path may include any number of bubble filters and/or traps to remove bubbles from the flow-path.

The interventional portion 104 may include a drive table that is configured to receive (e.g., be magnetically coupled to) any number of hubs (e.g., the first catheter hub 124a, the second catheter hub 124b, the third catheter hub 124c, the guidewire hub 124d, etc.). Advantageously, in some embodiments the hubs have no physical engagement with the drive table as they are magnetically coupled, through a sterile barrier, to carriages of the drive table thus eliminating any mechanical contact with the drive table. Instead, is such embodiments the hubs can move along a sterile barrier in which they may contact the sterile barrier but are not engaged (i.e., mechanically connected to, and not driven by a mechanical connection) to the drive table. Additional details of the hubs, drive table and related systems are found in U.S. patent application Ser. No. 17/816,669, entitled Method of Supra-Aortic Access for a Neurovascular Procedure, filed Aug. 1, 2022, which is hereby expressly incorporated by reference in its entirety herein. Each hub is configured to be coupled to a catheter, or guidewire, one or more fluidics lines, one or more electrical lines, one or more controls, and/or one or more displays. For example, a drive table may be positioned over or alongside the patient, and configured to support axial advancement, retraction, and in some cases rotation and/or lateral deflection of two or three or more different (e.g., concentrically or side by side oriented) devices (e.g., catheters, guidewires, etc.).

The drive system independently drives movement of each hub independently in a proximal or distal direction across the surface of the table to move the corresponding interventional device (e.g., the first catheter 126, the second catheter 128, the third catheter 130, and/or guidewire 132) proximally or distally within the patient's vasculature.

The respective first catheter 126, second catheter 128, third catheter 130, and/or guidewire 132 may be guided into a bodily lumen (not shown) as a single concentric catheter stack, in response to movement of the respective catheter hubs 124a-124c as discussed elsewhere herein. The fluidics system 100 may also include a guidewire hub 124d for controlling the guidewire 132, which may also be introduced into a bodily lumen along with one or more of first catheter 126, second catheter 128, and/or third catheter 130.

In some embodiments, a driven magnet is provided on each hub. Each driven magnet is configured to cooperate with a drive magnet associated with the table such that the driven magnet(s) move in response to movement of the drive magnet(s). In such examples, the drive magnet(s) may be axially movably carried by the support table.

Because multiple sources and/or sinks are configured to each be coupled (and remain coupled) to each catheter hub (e.g., the first catheter hub 124a, the second catheter hub 124b, and the third catheter hub 124c), the fluidics system 100 provides the advantage of enabling faster procedures than conventional fluidics systems that utilize manual removal, addition, and/or switching of fluids, catheters, hubs, and the like during the procedure. For example, the fluidics system 100 enables each fluid line/catheter hub to be connected to each source fluid and/or sink before beginning a procedure. When the interventionalist (or other medical practitioner) performing the procedure is ready to use a particular source fluid or sink, the fluidics system 100 is already configured and ready to allow use of the particular source fluid or sink without having to switch between different fluid lines for particular catheters. In some embodiments, the fluidics system 100 may be used to provide a method of treatment in which fluid sources need not be connected to and/or disconnected from a medical device more than once during a procedure.

Thus, the interventionalist can inject any of the fluids contained in fluid sources (e.g., the first source 110a and the second source 110b) and/or collect aspirate from any of the catheters 126, 128, and/or 130 at any point during the procedure because each catheter hub 124a-124c is provided access to all fluid lines at all times.

Because the multiple sources that are indicated for a particular procedure are preconfigured to be connected to each catheter/catheter hub, an interventionalist (or other medical practitioner) may be assured that there is no repetitive connecting and disconnecting of syringes or other source fluid containers, fluid lines, etc. during the procedure. This assurance removes the possibility of introducing bubbles into the catheter flow during the procedure because no connecting or disconnecting of fluid sources are needed with the use of the fluidics system 100. Instead, each fluid source and sink are connected and tested before the procedure and are not removed until after the procedure is completed. In some embodiments, the constant connection of fluid sources and sinks to catheter hubs associated with operation of the fluidics system 100 removes the variability and risk in remote procedures where the interventionalist is in a control room rather than the procedure room.

The valves within valve arrays 116a-116c of the fluidics system 100 are depicted at the respective manifolds 118a-118c. In such a configuration the valves are near the respective source and/or sinks with about two meters to about three meters (e.g., about six to about ten feet) of fluid line between the valves of valve arrays 116a-116c and the respective catheter hubs 124a-124c. In some embodiments, the valves of valve arrays 116a-116c may instead be located at the sources/sinks (e.g., first source 110a, second source 110b, and/or sink 112). In some embodiments, the valve arrays 116a-116c are coupled to the fluid lines at a location between the source/sink and the hubs. In some embodiments, one or more of the valve arrays 116a-116c may be located at the catheter hubs 124a-124c. In some embodiments, valves that are located at or near the hubs may be disposable valves. Other components of the fluidics systems 100, 200 may also be disposable and/or re-processable for reuse.

In some embodiments, the fluidics system 200 may additionally include check valves 113a, 113b, 113c, 113d, 113e, 113f, 113g, 113h, 113i (hereinafter “check valves 113a-113i”) between the valve arrays 116a-116c and the respective catheter hubs 124a-124c. The check valves 113a, 113b, 113c can be part of a valve manifold (for example, such as the valve manifold 16 of FIG. 1B) that is part of or coupled, directly or indirectly, to the first catheter hub 124a. The check valves 113d-113f can be part of a valve manifold (for example, such as the valve manifold 16 of FIG. 1B) that is part of or coupled, directly or indirectly, to the catheter hub 124b. The check valves 113g-113i can be part of a valve manifold (for example, such as the valve manifold 16 of FIG. 1B) that is part of or coupled, directly or indirectly, to the third catheter hub 124c. The check valves 113a-113i may be one-way check valves. As shown in FIG. 2, the one-way check valves 113a-113i can allow flow in the direction in which their respective arrows are pointing.

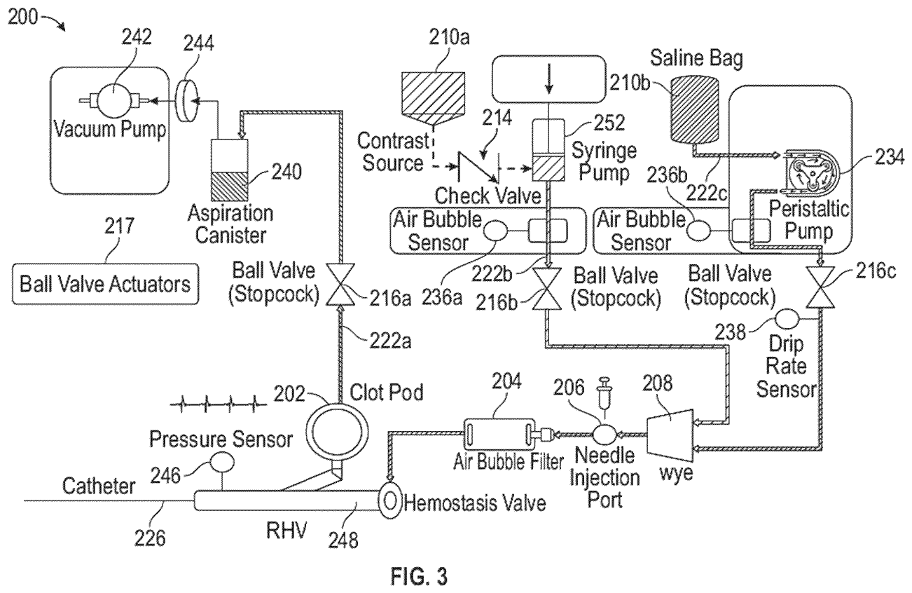

Each of the catheter hubs 124a-124c may be provided with a hemostasis valve to accommodate introduction of another device therethrough, as illustrated in FIG. 3. The hemostasis valve includes a variable diameter aperture such as an aperture through a resilient gasket.

The gasket may be actuatable between a first, fully open state; a second partially open state for sealing against low pressure fluid injections from the first fluid source or the second fluid source through a first port (as described herein), for permitting fluid to flow through the first port to the sink, while allowing advancing or retracting an interventional device; and a third tightly closed state for resisting backflow of high pressure fluid (e.g., contrast media) injections from the second fluid source through the first port or for permitting fluid flow through the first port to the sink. The gasket may be manually actuatable or automatically actuatable, for example based on a user input corresponding to manipulation of one or more of the interventional devices of the system.

FIG. 3 illustrates another embodiment of a fluidics system 200 for use with a fluidics management system. In general, the fluidics system 200 includes two or more fluid channels that feed one or more fluid lines configured to interface with a rotating hemostatic valve. The two or more fluid channels may receive fluids from fluid sources with any number of fluid materials. The two or more fluid channels may allow different fluid materials provided by different fluid sources (of various volumes and/or under different pressures) to flow into or from a bodily lumen. Each of the two or more fluid channels may be primed with a respective fluid in order to be ready for transport into the single fluid line and into the body. Valves and/or valve arrays may be employed to switch between usage of the two or more fluid channels.

As shown in FIG. 3, the fluidics system 200 may include a sterile field clot capture container, vacuum chamber, and/or control 202, within the sterile field. The vacuum chamber may have a clot filter with a window for visualizing trapped clot, and a valved vent which when momentarily opened permits entrance of air to allow direct visualization of the clot through the window. To permit inspection by a remote physician, a CCD or CMOS sensor may be mounted such that the upstream surface of the clot filter is within the sensor field of view. This permits viewing the contents of the filter on a remote monitor. Air intake to clear the optical path from the window to the filter may be controlled remotely using an electronically actuated valve.

An air bubble filter 204 may be provided in line between a needle injection port 206 and catheter 226. The fluidics system 200 further includes a line branch point 208 (e.g., a wye) in fluid communication with a first source 210a and a second source 210b. The line branch point 208 may include luer lock connectors or wye connector that interfaces with multiple fluid sources.

The fluidics system 200 may also include a pump 234 such as a peristaltic pump or rotary piston pump that drives fluid under pressure from the second source 210b to the line branch point 208 in the direction of arrow 222c.

An air bubble sensor may be provided on an upstream or downstream side of the pump 234. The air bubble sensors 236a, 236b may be non-contact ultrasonic flow sensors that measure an intensity and doppler shift of a reflected ultrasound through a sidewall of fluid tubing to detect bubbles and measure fluid flow rates or fluid levels as has been discussed. In some embodiments, the air bubble sensor 236a may also be a pressure sensor or a separate pressure sensor may be provided.

A third valve 216c such as a ball valve or rotary valve can selectively open or close fluid communication between the second source 210b and the catheter 226. A flow rate detector such as a drip rate sensor 238 enables determination and display of the flow rate from second source 210b.

Fluid flow from the first source 210a is directed through a one way check valve 214 and on to a high pressure pump 252, which may be a syringe pump, high pressure positive displacement pump, contrast injection pump, etc. High pressure fluid (e.g., contrast solution) is directed through an air bubble sensor 236a and on to the line branch point 208 through the second valve 216b. Arrows 222b indicate the direction of fluid flow.

Resistance to fluid flow through different catheters in a concentric catheter stack differs based upon the available lumen cross sectional area. For example, resistance measurements within an inner catheter with a fully open lumen (e.g., with guidewire removed) may be lower than resistance measurements within an outer catheter having a second catheter (or a guidewire) extending therethrough. Therefore, when performing saline flushing steps, the fluidics system 200 may be configured to ensure a similar flow rate or a procedure appropriate flow rate through each inner and outer catheter to avoid clotting or other issues within the catheters. To do so, a valve may be adjusted for each catheter to ensure the flow rate remains constant amongst all catheters during saline flushing. The fluidics system 200 may determine such flow rates in real time based on flow rate sensors, and the control system may be configured to automatically adjust valve settings and/or pump parameters to maintain the desired flow rate through each catheter.

In some embodiments, fluid resistance may be altered by adjusting an insertion length of each shaft into its concentrically adjacent lumen. As described herein, fluid resistance within a lumen may be greater when there is a reduction in cross sectional luminal area for flow, for example, when a second catheter (or a guidewire) extends into the lumen. The amount of fluid resistance can be affected by the length of the cross sectional narrowing, for example, due to placement of the second catheter (or guidewire) within the lumen. A second catheter (or guidewire) extending partially through the lumen of a first catheter will provide a smaller length of cross-sectional narrowing, and accordingly may result in a lower fluid resistance within the lumen of the first catheter, than if the second catheter (or guidewire) were to extend entirely through the lumen of the first catheter. Thus, fluid resistance can be lowered by partially retracting a depth of insertion of a second catheter (or guidewire) into the lumen through which fluid is to be injected.

The fluidics system 200 further includes an aspiration canister 240 coupled to an upstream side of filter 244. A downstream side of the filter 244 is coupled to a vacuum pump 242. The aspiration canister 240 is connected to a first valve 216a, which may be in communication with a sterile field clot capture container, vacuum chamber, and/or control 202 which has been discussed elsewhere herein. Arrow of the fluid lines 222a indicates the direction of fluid flow.

An optional pressure sensor 246 is depicted on a proximal end of a catheter 226 or hub coupled to a hemostatic valve, such as a rotating hemostatic valve (RHV) 248. In other embodiments, different types of hemostatic valves (e.g., non-rotating hemostatic valves) may be used alternatively to the RHV 248.

In this example, the RHV 248 is connected to two different fluid sources. The RHV 248 may be carried by and at least partially disposed in a hub (e.g., the first catheter hub 124a of FIG. 2). The RHV 248 may comprise a first fluid source connection, a second fluid source connection, and a sink connection. For example, the first connection, the second connection, and the third connection may comprise respective valves (e.g., valves 216a, 216b, 216c) connected via fluid lines to RHV 248. In some embodiments, the connection points may be formed as part of the RHV 248 itself and fluid lines may connect directly to the connection points at proximal ends of the fluid lines and connect to sources and/or sinks at the respective distal ends of the fluid lines. In certain embodiments, the valves 216a, 216b, 216c may be arranged in a valve manifold or a valve manifold cassette. In certain embodiments, the pump 234 may also be arranged in the valve manifold or valve manifold cassette. In certain embodiments, any of the valves 216a, 216b, 216c can be a ball valve, a stopcock valve, a rotary valve, a solenoid valve, or any other suitable valve. Any of the valves 216a, 216b, 216c can be controlled by one or more actuators 217.

The RHV 248 may be configured to enable a catheter or other instrument to be introduced into the body of a living being while precluding unintended back bleeding. In some embodiments, each RHV described herein may be configured with at least a fully closed configuration, a low sealing force state in which devices may be advanced therethrough without leaking, and a high sealing force state (e.g., mode) which prevents escape of fluids under high pressure and may prevent axial movement of devices therethrough.

The RHV 248 is configured to be concurrently and fluidly connected to a first fluid source (e.g., the first source 210a) via the first fluid source connection (e.g., the second valve 216b). The RHV 248 is further configured to be concurrently and fluidly connected to a second fluid source (e.g., the second source 210b) via the second fluid source connection (e.g., the third valve 216c). In addition, the RHV 248 is further configured to be concurrently and fluidly connected to the sink (e.g., aspiration canister 240) via the sink connection (e.g., the first valve 216a).

In operation, the fluidics system 200 is configured to automatically switch between introducing fluid into a lumen of the elongate body (e.g., catheter 226) through the RHV 248 from the first fluid source (e.g., the first source 210a) or from the second fluid source (e.g., the second source 210b) or to permit fluid removal from the lumen to be collected in the sink (e.g., aspiration canister 240).

In some embodiments, the optional pressure sensor 246 is located at either the upstream side or downstream side of the RHV 248 (as shown in FIG. 3). In some embodiments, the optional pressure sensor 246 is located in the catheter (e.g., in a sidewall of the catheter) to measure arterial pressure at the catheter distal end. The pressure may be assessed by the interventionalist to verify that the catheter is not misaligned within the vessel and/or the thrombus.

For example, if the catheter is misaligned against a vessel wall, then the detected pressure (e.g., waveform) may be blunted. Such a detection may be provided to an algorithm performed by a processor associated with the fluidics system 200, for example, to determine the patency of the lumen of the catheter of the patency of the catheter distal tip. Such a pressure sensor and algorithm may provide an improved alternative to conventional determinations of pressure where manual operation of fluidics is occurring and an interventionalist may retract (e.g., pull back) on a syringe coupled to the catheter to verify that blood capture occurs and to assess tactile feedback of the catheter.

Such blood capture and tactile feedback assessments may indicate patency of the lumen or distal tip before an injection or aspiration is performed. However, the pressure sensor 246 may provide for an automated and improved way to assess lumen or distal tip patency. That is, the addition of a pressure sensor 246 (e.g., a blood pressure sensor) on the proximal end of a catheter may capture an arterial pressure waveform. The waveform can be used to determine whether the catheter distal tip is pressed against a vessel wall, the catheter tip is pressed against a thrombus, the catheter tip has full patency, or the catheter lumen is in a clogged or fully patent state, without having direct visual or tactile feedback. In some embodiments, the waveform can be used to determine a state of engagement of the catheter distal tip against the clot and/or a consistency of the clot.

In some embodiments, the fluidics systems (e.g., fluidics system 100, fluidics system 200) described herein include a hemostasis valve (e.g., RHV 248) that includes a first three-way connector having a first fluid source connection (e.g., first valve array 116a, or second valve 216b), a second fluid source connection (e.g., second valve array 116b, or third valve 216c) and a sink connection (e.g., the first valve 216a).

In some embodiments, the fluidics systems described herein (e.g., fluidics system 100, fluidics system 200) utilize a first fluid source that comprises one of saline, heparinized saline, or a pharmaceutical. In some embodiments, the second fluid source (e.g., second source 110b, 210b) comprises contrast.

The fluidics systems 100, 200 may further include a second hemostasis valve that is in communication with and may be at least partially disposed in the second hub (e.g., the second catheter hub 124b). The second hemostasis valve may include a third fluid source connection (e.g., second valve array 116b), a fourth fluid source connection (second valve array 116b), and a second sink connection (e.g., third valve array 116c). In this example, the first manifold 118a may include a second output line that is configured to connect to a third fluid source connection (not shown).

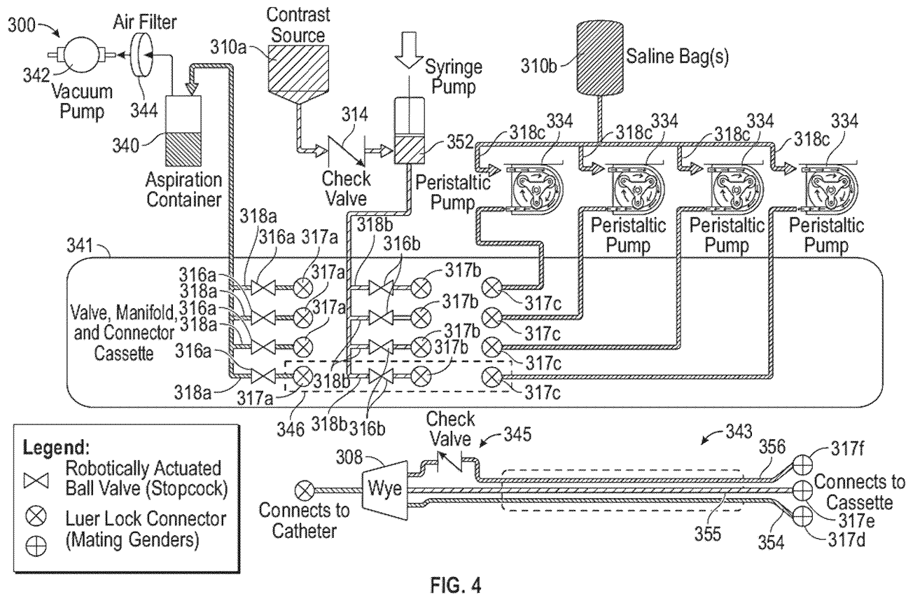

FIG. 4 illustrates another embodiment of a fluidics system 300 for use with a fluidics management system. In general, the fluidics system 300 includes a cassette that can couple a plurality of fluid sources and/or sinks to a plurality of interventional devices. A plurality of fluid lines may extend between a source or sink and the cassette for coupling to different interventional devices. For other sources and sinks, a single fluid line may extend between the source or sink and the cassette and may split within the cassette to connect to different interventional devices. In certain embodiments, the cassette can include connection arrays formed of connections from a plurality of fluid sources and/or sinks for coupling to a single interventional device (for example, in a row or column). Each connection array can couple to a tubing set having a tube corresponding to each connection in the connection array.

The cassette 341 may be a self-contained unit comprising a housing having a plurality of valves, tubing and connectors as described below. A first connector array comprises a plurality of releasable connectors such as luer connectors, for placing the cassette in fluid communication with complementary connectors in fluid communication with sources of aspiration and at least one or two or more fluids. A second connector array is configured for releasable connection to a tubing set configured to extend between the cassette and at least one or two or three interventional devices.

The cassette 341 thus forms a bridge module that when assembled resides between the various fluid and vacuum sources, and the corresponding interventional devices. The cassette 341 may be configured for a single use, or may be re-sterilizable and reusable.

As shown in FIG. 4, the fluidics system 300 may include a first fluid source 310a and a second fluid source 310b. Fluid flow from the first fluid source 310a is directed through a one way check valve 314 and on to a high pressure pump 352, which may be a syringe pump, high pressure positive displacement pump, contrast injection pump, etc. The fluid from the first fluid source 310a may be a contrast solution which is preferably injected under high pressure.

Fluid flow from the syringe pump is directed into a cassette 341, which may include a plurality of valves, manifolds, and/or connectors. Within the cassette 341, the fluid flow may split along a plurality of branches 318b to a plurality of connectors 317b (for example, four connectors 317b as shown in FIG. 4.) for coupling with different interventional devices. The cassette may include a valve 316b (e.g., a ball valve) with each branch 318b upstream of the connector 317b. In certain embodiments, any of the unique valves 316a, 316b can be a ball valve, a stopcock valve, a rotary valve, a solenoid valve, or any other suitable valve.

Fluid flow from the second fluid source 310b may be directed into a plurality of branches 318c to a plurality of pumps 334 (for example, four pumps 334 as shown in FIG. 4), such as peristaltic pumps or rotary piston pumps. Each of the plurality of pumps 334 can drive the fluid (for example, saline) under pressure from the second fluid source 310b to a unique connector 317c for each interventional device within the cassette 341.

The system further includes an aspiration canister 340 in communication with an upstream side of a filter 344. A downstream side of the filter 344 is in communication with a vacuum pump 342. The aspiration canister receives fluid from the cassette 341 which includes a plurality of connectors 317a each being configured to couple to a unique interventional device. A unique valve 316a (at least two, and four in the illustrated example) may be positioned upstream of each of the plurality of connectors 317a. Each unique valve 316a may be positioned along a branch 318a.

In certain embodiments, one or more connector arrays 346 may be arranged, each connector array 346 configured to couple an interventional device. For example, a connector array 346 is indicated by dashed lines in FIG. 4. As shown in FIG. 4, the connector array 346 can include one of the plurality of connectors 317a, one of the plurality of connectors 317b, and a connector 317c. As shown in FIG. 4, the connector array 346 may be organized with all connectors facing in the same direction on a common plane, such as a linear row.

The connector array 346 can releasably couple to a tubing set 343 including an aspiration tube 354, a first fluid tube 355, and a second fluid tube 356. In some embodiments, the plurality of connectors 317a, 317b, and 317b can be luer lock connectors. The aspiration tube 354 can couple to one of a plurality of connector 317a of the connector array 346 by way of a complementary connector 317d for aspiration from the interventional device to the aspiration container. The first fluid tube 355 can couple to one or a plurality of connectors 317b of the connector array 346 by way of a complementary connector 317e to provide fluid flow from the first fluid source 310a to the interventional device. The second fluid tube 356 can couple to the connector 317c of the connector array 346 by way of a complementary connector 317f to provide fluid flow from the second fluid source 310b to the interventional device. The tubes 354, 355, 356 may be joined together over a majority of their lengths. The tubes 354, 355, 356 can each have a length of at least about three or four feet, and in certain embodiments between about 6 feet and about 8 feet.

As shown in FIG. 4, the tubing set 343 includes a line branch point 308 (e.g., a two to one or a three to one wye) that can provide fluid communication between the interventional device and the aspiration tube 354, the first fluid tube 355, and second fluid tube 356. The line branch point 308 may include luer lock connectors or wye connectors that interface with complementary connectors on the tubing set. In certain embodiments, a one way valve 345 may be positioned upstream of the line branch point 308 and downstream of the cassette 341 along the flow-path of the second fluid.

In certain embodiments, the fluidics system 300 (or other systems described herein) can direct the flow of the second fluid (for example, saline) using two different flow modes. In a low flow drip mode, a flow rate of about 1-2 drips per second or 3-6 mL/min may be provided, for example, by the plurality of pumps 334. In some embodiments, a low flow mode rate of 1-8 mL/min may be provided. Each catheter coupled to the system may experience a different fluid resistance as described herein.

The pumps, for example the plurality of pumps 334, can be operated to provide the same flow rate in each catheter. In certain embodiments the fluid pressure within the catheter can be at least about 330 mmHg or 6.5 psi. This pressure may be enough to overcome arterial pressure while delivering the desired drip rate. In certain embodiments, the pressure within the catheter can be greater than 330 mmHg. In certain embodiments, the delivered fluid volume can be at least about 1 liter over the length of a procedure. In some embodiments, the fluid volume can be up to 2 liters.

In a high flow flush mode, all of the fluid lines may be flushed to remove air. The flow rate can be between 100-1000 mL/min. The fluid pressure may be between 5-10 psi. The volume delivered can be between 0.5-1 liters per procedure. Volume may depend on tubing length and diameter. In some embodiments, the high flush flow rate is at least about 20 times and in some cases between 30 to 150 times the low flow drip mode flow rate.

In certain embodiments, the first fluid (for example, contrast solution) can be provided at a flow rate of between 3-8 L/s (for example, about 4 mL/s), for example, by the pump 352. In certain embodiments, the flow rate can be up to about 8 mL/s. In other embodiments, the flow rate can be up to about 20 mL/s. In certain embodiments, the first fluid can be provided with a pressure of about 400 psi for a flow rate of about 4 mL/s. The amount of pressure needed may depend on flow rate and flow restriction of the fluid path. The pressure may increase proportionally with the flow rate for higher flow rates. In certain embodiments, the pressure may be up to 1200 psi.

In certain embodiments, the high pressure pump, such as pump 352, can provide a delivered volume of between 5-15 mL per high pressure injection. In certain embodiments, the pump can provide the 5-15 mL per high pressure injection in increments of about 1 mL per puff. In certain embodiments, the second fluid source can provide a total volume of about 200 mL per procedure. In certain embodiments, the syringe pump is sized to hold at least about 150 mL or 200 mL so as to provide uninterrupted flow throughout the procedure without the need to add additional contrast solution. In other embodiments, the second fluid source can provide a total volume of between 150-250 mL per procedure.

In certain embodiments, the flow rate may vary depending upon the anatomical location at the distal end of the catheter. For example, within the aortic arch, the flow rate may be about 20 mL/s. A total delivered volume of about 25 mL may be infused in the aortic arch. Within the common carotid artery, the flow rate may be about 20 mL/s. A total delivered volume of 12 mL may be infused in the common carotid artery. Within the subclavian artery, the flow rate may be about 6 mL/s. A total delivered volume of about 15 mL may be infused in the subclavian artery. Within the internal carotid artery, the flow rate may be about 6 mL/s. A total delivered volume of about 8 mL may be infused in the internal carotid artery. Within the external carotid artery, the flow rate may be about 3 mL/s. A total delivered volume of about 6 mL may be infused in the external carotid artery. Within the vertebral artery, the flow rate may be about 6 mL/s. A total delivered volume of 8 mL may be infused in the vertebral artery.

In certain embodiments, a motor may be provided to drive the high pressure pump, such as pump 352, which can be controlled with a position and velocity control loop using a potentiometer as a measurement to close the loop. In certain embodiments, current control may be applied to provide approximate pressure limiting. In certain embodiments, the second fluid can be a contrast solution such as Omnipaque 300, Omnipaque 350, or Visipaque 320.

In certain embodiments, a vacuum pump, such as vacuum pump 342, can provide a pressure of about −29.5 inHg or up to −29.5 inHg (-999 mbar). In certain embodiments, tubing used for aspiration can have an inner diameter of 0.11 inches (about 2.8 mm). In certain embodiments, the volume of the aspiration canister 340 can be at least about 0.5 L. In certain embodiments, the volume of the aspiration container can include about 0.5 L for blood and additional volume for a saline flush. In certain embodiments, the aspiration container can have a volume between 0.25-0.75 L. In certain embodiments, the vacuum pump can be configured to operate to additionally provide a low pressure/flow setting to assist a flushing process as it may be desirable that an aspiration line is full of saline at all times (except when aspirating a clot). In certain embodiments, a separate pump may be provided for the low pressure/flow setting.

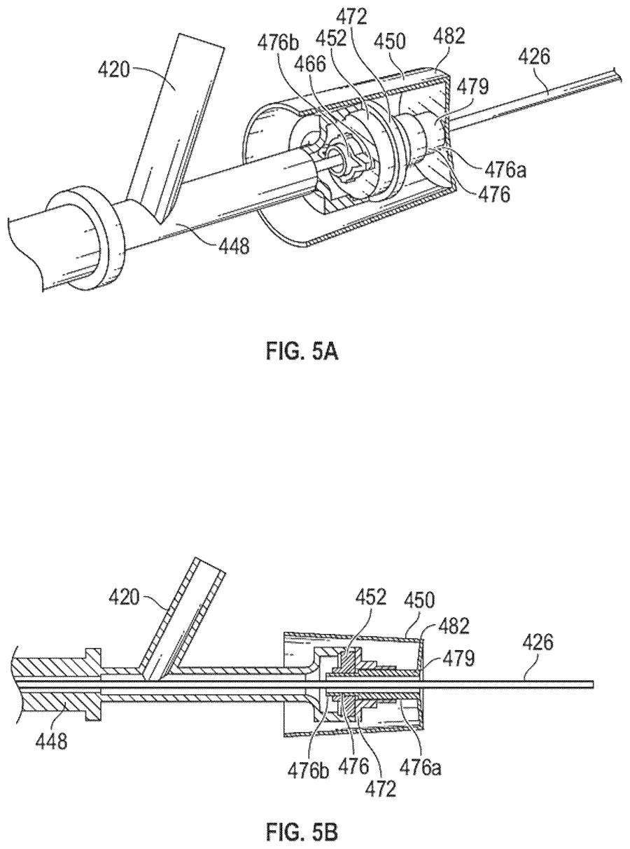

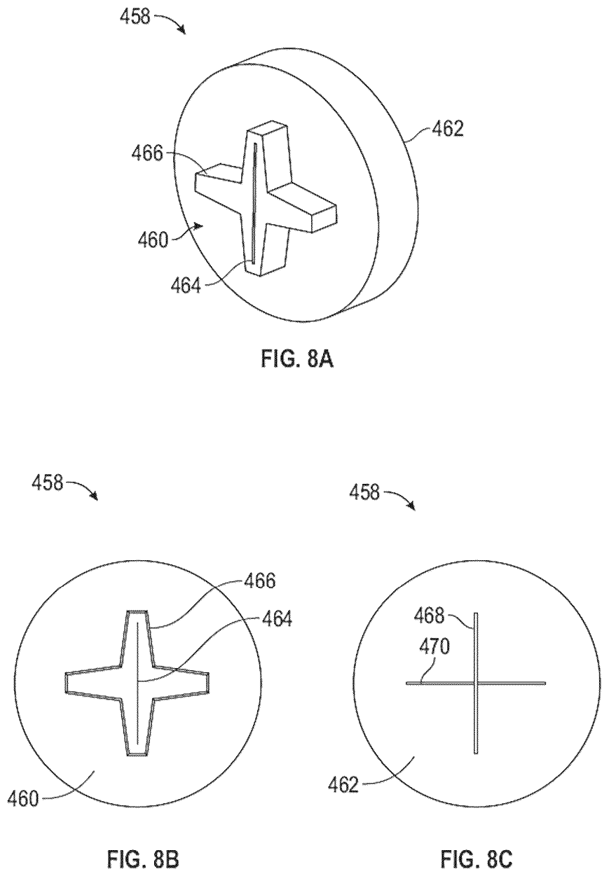

FIGS. 5A and 5B illustrate perspective and cross-sectional views of an example rotating hemostatic valve (RHV) 448 with a dual membrane gasket configured in an open position. That is, both a distal and a proximal membrane are provided with a slit, and a support tube is positioned through both membranes to hold the slits in an open, constrained configuration (e.g., see FIG. 8B). The RHV 448 may represent the RHV 248 in the fluidics system 200, or a hemostasis valve in any of the catheter hubs 124a-124c may be configured for use with a manual procedure and fluidics system, a robotic procedure and fluidics system, or a combination thereof.

The RHV 448 includes a side port 420, a dual membrane gasket 452, and a plunger 476 having a housing 482 and a support tube 479 configured to reversibly advance distally through the gasket to maintain patency therethrough. The RHV 448 is coupled to a proximal end of a first interventional device and is adapted to receive a second interventional device (e.g., a catheter 426) therethrough. The plunger includes a proximal end 476a and a distal end 476b.

The second interventional device is disposed in a lumen defined by the first interventional device. As shown, the catheter 426 is advanced through the support tube 479 of the RHV 448. A proximal end 450 of the RHV 448 includes a housing coupled to the plunger 476. The gasket 452 is configured to be coupled to a gasket housing 472 that surrounds a circumference of the plunger.

The gasket 452 may be actuatable between a first fully open state, a second low sealing force state for sealing around a catheter but permitting sliding movement of the catheter, a third state for sealing around a catheter for high pressure management and a fourth, completely closed state in the absence of any secondary devices extending therethrough.

The first open state represents a back bleed position or an interventional device loading or unloading position that configures the RHV 448 to allow the gasket 452 to be fully open. The second partially open state represents a position that configures the RHV 448 to allow the gasket 452 to close around the catheter 426 within the RHV 448 with sufficient sealing that blood or saline solution pumped at relatively low pressure does not leak while the catheter 426 is advanced or retracted through the RHV 448 with low resistance. The third state represents a tightly sealed configuration for enabling high pressure fluid (contrast) injection from a fluid source. In certain embodiments, a control system can be configured to determine a sealing force of the hemostasis valve around the catheter 426 (for example, in response to a human input). The control system can be configured to change the sealing force if it is determined that the sealing force is too high or too low. For example, the control system can increase the sealing force if the sealing force is too low.

As shown in FIG. 5B, the gasket 452 is restrained by the support tube in the first open state. The first open state ensures that the gasket 452 is configured in a back bleed position or an interventional device loading or unloading position. The open position configures the RHV 448 to open the gasket 452 by moving a tubular support portion of the plunger 476 through the gasket 452 such that the tubular support forcibly opens and restrains the gasket providing an open central lumen. The movement may allow arterial blood pressure to push blood proximally through the catheter 426 until the blood is viewable at the RHV 448. Moving the gasket into the open position, which enables back bleeding, allows visual and/or tactile confirmation that there are no air bubbles in the RHV 448 and/or that the distal tip of the catheter is not against a vessel wall and/or that a lumen defined by the catheter is unobstructed.

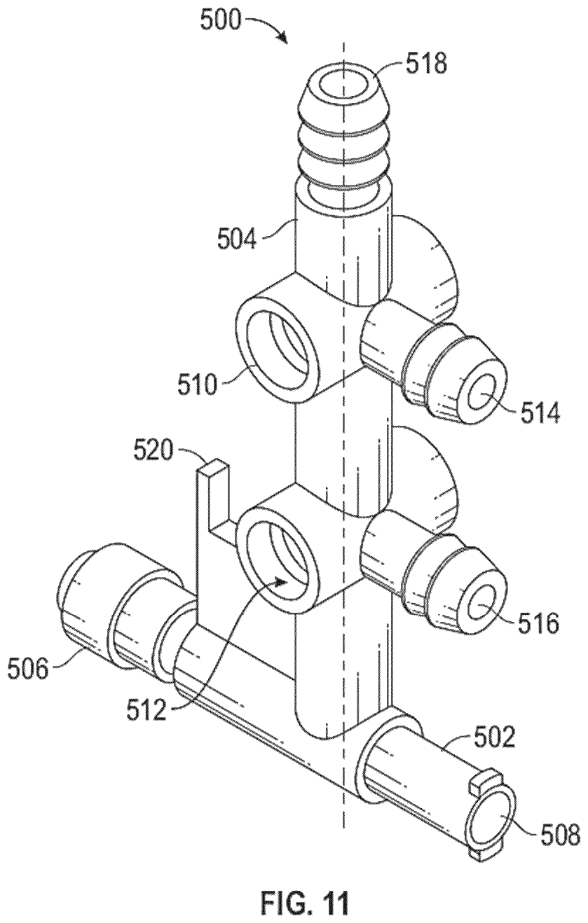

In some embodiments, the RHV 448 may include a side port 420. The side port 420 may be releasably connected to a three-way connector that is configured to be fluidly connected to a first fluid source (e.g., first source 110a), a second fluid source (e.g., second source 110b), and a sink (e.g., sink 112). Alternatively, or additionally, the RHV 448 may further include one or more additional ports for connection with fluid sources and/or sinks. See, for example, FIG. 11 which will be described in further detail elsewhere herein.

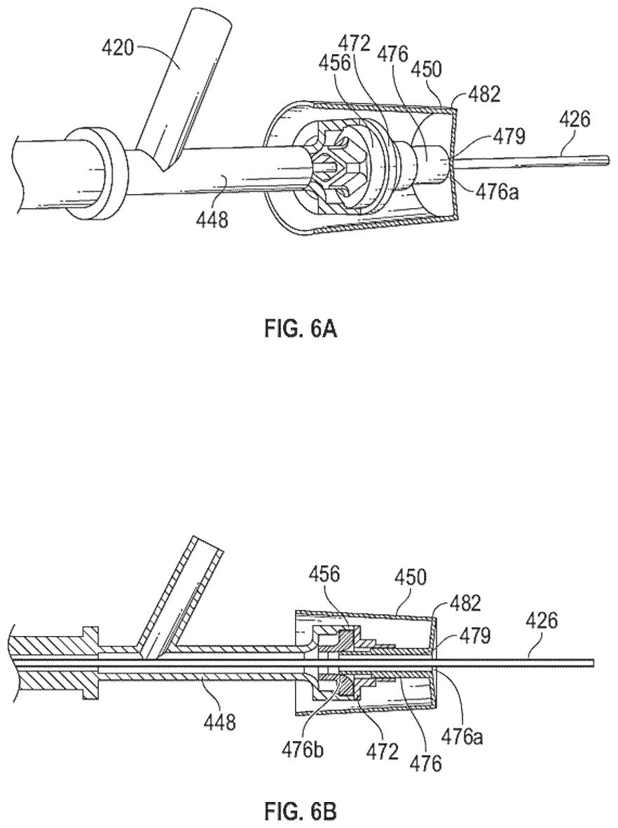

FIGS. 6A and 6B illustrate cross-sectional and zoomed in cross-sectional views of a rotating hemostatic valve with a gasket (e.g., gasket 456) configured in a partially open or low sealing pressure position. In this position, the distal end 476b of plunger 476 has been retracted proximally such that the distal slit 464 of the gasket 456 (see FIG. 8B) is in sliding contact with catheter 426 but the proximal slit 468, 470 (see FIG. 8C) remain restrained in the open configuration by the support tube. The low pressure state of the gasket 456 enables saline (e.g., from second source 110b) to be burst injected through the RHV 448 side port 420 at a pressure of up to about 276 kPa (i.e., about 40 psi).

As shown in FIG. 6B, the gasket 456 is in the partially open state with the plunger 476 advanced partially into the gasket 456. This position represents a low pressure position in which blood or saline solution does not leak while the catheter 426 is advanced or retracted through the RHV 448 with low resistance. The second partially open state configures the RHV 448 for receiving low pressure fluid injections from a first fluid source (e.g., first source 110a) or a second fluid source (e.g., second source 110b) through a first port or for permitting fluid to flow through the first port to a sink (e.g., sink 112).

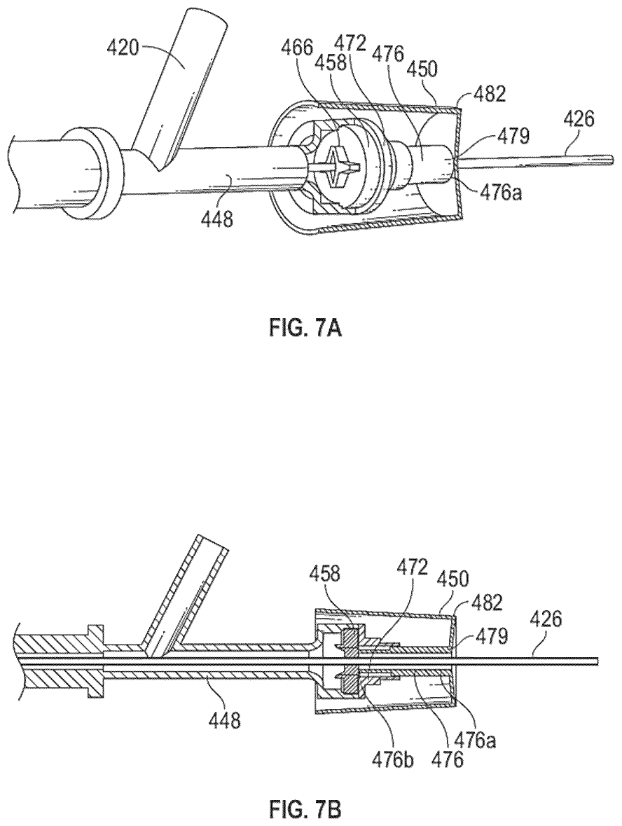

FIGS. 7A and 7B illustrate cross-sectional views of an example rotating hemostatic valve with a gasket configured in a high sealing force (mode) or high-pressure position. As shown in FIG. 7A, a distal membrane 460 includes a thickened sidewall in the form of a cross of material 466 which aligns with the transverse slits of the proximal membrane 462. The cross of material 466 provides additional gasket contact around the catheter 426 in the tightly closed (e.g., high-pressure) state.

The tightly closed state represents a configuration for high-pressure fluid transfer from a fluid source (e.g., contrast/second source 110b). The closed state of the gasket 458 enables contrast medium (e.g., from second source 110b) to be injected through the RHV 448 side port 420 at a pressure of up to about 2.76 MPa (i.e., about 400 psi). As shown in FIG. 7B, in the closed state, the plunger 476 is retracted proximally so that both the distal slits 464 and proximal slits are no longer supported by the support tube and are able to provide a seal against the catheter 426. In other words, both the proximal end 476a and the distal end 476b of the plunger 476 are clear of the gasket 458 to achieve the closed state of the gasket 458.

FIGS. 8A-8C illustrate various views of an example gasket for use in the fluidics systems described herein. FIG. 8A illustrates a perspective view of the gasket 458. The gasket 458 includes a distal membrane 460 and a proximal membrane 462. A cross slit (substantially horizontal slit 470 intersecting substantially vertical slit 468) on the proximal membrane 462 of the gasket 458 may be allowed to compress against the catheter for high-pressure sealing or a closed position of gasket 458. A distal slit 464 vertically oriented on a distal membrane 460 of gasket 458 may be allowed to compress against the catheter for low-pressure sealing or a closed position of gasket 458.

FIG. 8B illustrates a distal end view of the gasket 458. The distal membrane 460 of the gasket 458 includes the distal slit 464 vertically oriented in a center of a gasket portion having the thickened sidewall which may be configured as a cross of material 466.

FIG. 8C illustrates a proximal end view of the gasket 458. A vertical slit 468 of the gasket 458 is substantially perpendicular to a horizontal slit 470 on the proximal membrane 462 of the gasket 458.

In some embodiments, the rotating hemostatic valves described herein may be configured with an open setting during which catheters can be freely inserted or removed from the lumen manually. In addition, in the open setting, free flushing of the system with saline may be performed to purge the system of air bubbles. In some embodiments, the open setting may additionally allow for retrograde back bleeding of blood to purge the system of air bubbles.

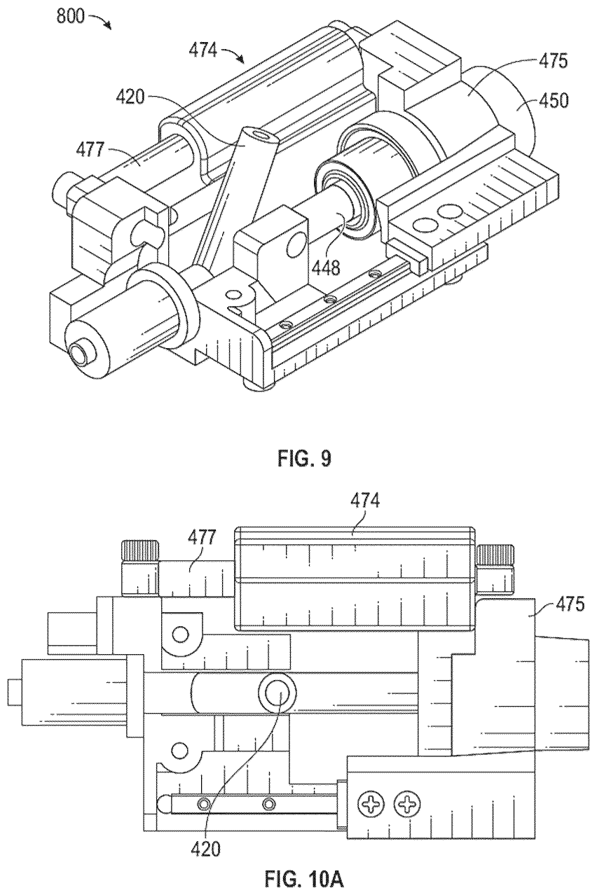

FIG. 9 illustrates a perspective view of an actuation mechanism 800 for use with the rotating hemostatic valves described herein. The actuation mechanism 800 comprises at least a linear actuator 474 connected coaxially about a tubular drive shaft 477, which couples to the proximal end 450 of the RHV 448 to drive one or more gears to rotate the RHV 448. To manipulate the catheter (e.g., catheter 226), the linear actuator 474 drives a tang (not shown) which may engage with a circular flange fixed to an outer shaft of a catheter. In some embodiments, the RHV 448 is coupled to a hub (e.g., the first catheter hub 124a) capable of rotation, translation, and/or deflection of a catheter (or wire).

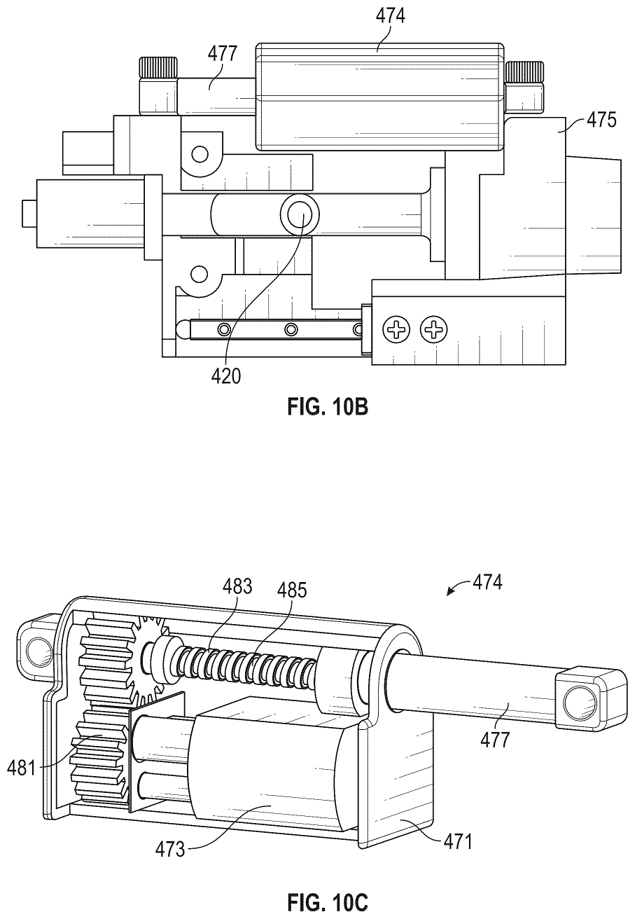

FIG. 10A illustrates the RHV drive mechanism restraining the valve in the open position as illustrated in FIG. 5B. The linear actuator 474 has driven the drive shaft 475 to a proximal limit of travel, at which the support tube extends across the gaskets, holding them open. In FIG. 10B, the support tube has been advanced out of the gaskets, allowing them to close tightly around any inner catheter extending therethrough in the high pressure position.

FIG. 10C is a schematic representation of one implementation of a linear actuator 474. A housing 471 supports a motor 473 which rotates a gear train 481 which in turn rotates a lead screw 483. A helical thread 485 on lead screw 483 slidably engages a complementary projection on an inside surface of a tubular drive shaft 477, causing axial reciprocation of the drive shaft 477 and corresponding axial displacement of the support tube relative to the gaskets.

The sterile field clot capture container, filter, RHV, pressure sensor, etc. could all be part of the hub and move with the catheter. If capturing the clot with the clot pod close to the hub, then the tubing between the hub and the fluidics management tower does not need to be as large of a diameter (would inject saline at higher pressure than arterial pressure and then aspirate back through clot pod to make the clot more visible). The valve manifold may be configured without regard to design details for handling large pieces of clot going through the manifold. In some embodiments, the valve manifold may be carried by the hub. In some embodiments, the valve manifold may be integrated into the hub. Alternatively, the valve manifold may be remote from the hub, and in communication with the hub by way of a tubing set having vacuum, saline, and contrast lines.

If the first catheter 126 is left in place, pulling out second catheter 128 creates a pressure gradient from outside to inside, creating a risk of sucking in air if the valve isn't tight enough, but the valve can't be so tight that it inhibits pulling out the second catheter 128, so the saline delivery flow rate may be set so that it is creating a positive pressure so that no air bubbles are introduced.

FIG. 11 illustrates a perspective view of an example assembly 500 that includes a rotating hemostatic valve (RHV) 502 integrated with a manifold 504. The example shown in FIG. 11 depicts an embodiment with a manifold located adjacent to or within a particular RHV associated with a hub. Such an embodiment may replace the separate manifolds 118a-118c in the fluidics system 100.

The RHV 502 is depicted as a tapered tube with a rotating portion 506 and a port 508 for receiving one or more catheters (not shown) threaded therethrough. One or more catheters may be attached to a portion of RHV 502 adjacent to port 508. The portion may include a luer lock rotating nut or another valve or introducer valve.

The RHV 502 may be fixedly attached to the manifold 504. In some embodiments, the RHV 502 is removably attached to the manifold 504. The manifold 504 is configured with any number of ports for receiving fluid lines attached into hubs. For example, the manifold 504 includes at least a first port 510 for receiving one or more fluid source lines 120a, 120b, 120c from at least one manifold valve (e.g., at least one valve in the first valve array 116a). The manifold 504 includes at least a second port 512 for receiving one or more fluid source lines 121a, 121b, 121c from at least one manifold valve (e.g., at least one valve in the second valve array 116b).

The manifold 504 is further configured with any number of ports for receiving fluid lines connected to particular fluid sources. For example, the manifold 504 includes a port 514 for receiving contrast fluid via fluid lines connected to a contrast source (e.g., second source 110b). The manifold 504 also includes a port 516 for receiving saline fluid via fluid lines connected to a saline source (e.g., first source 110a). The manifold 504 additionally includes a port 518 for receiving (e.g., evacuating) waste via fluid lines connected to a sink (e.g., sink 112). The manifold 504 may include an optional clip 520 for attaching the RHV 502 to a hub.

Although two source ports and a sink port are depicted in FIG. 11, any number of source ports or sink ports may be possible on the assembly 500. Further, any number of valve ports may also be provided and may correspond to a number of catheter hubs configured to function in a particular fluidics system.

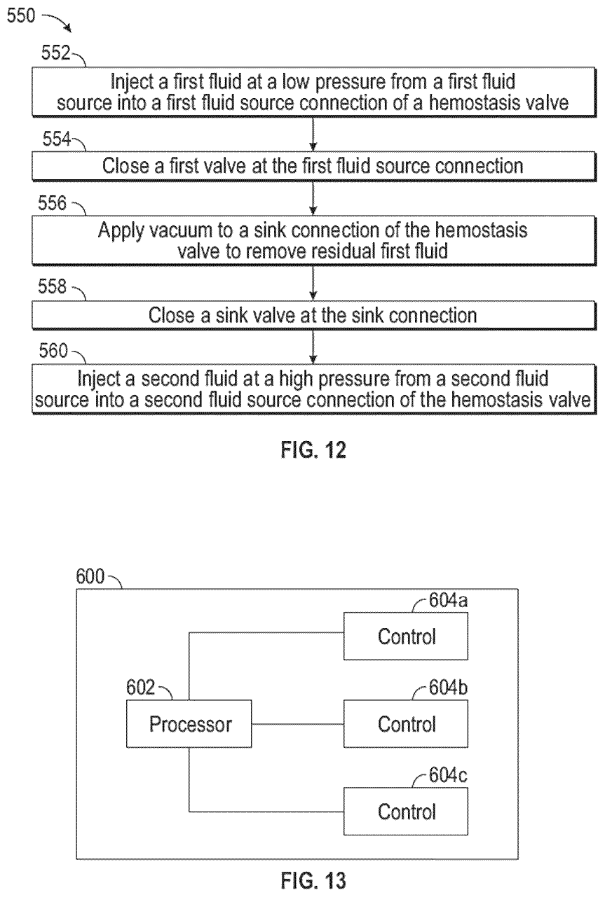

FIG. 12 depicts an example of a method 550 for degassing a fluid management system of a robotically driven medical device. Embodiments of the method for degassing a fluid management system may remove gases from a lumen of robotically driven medical device (e.g., a catheter as described herein), one or more fluid lines of the fluid management system, and/or or one of more fluids (for example, removing dissolved gases from one or more fluids). In certain embodiments, the method for degassing may be used to build a wall or column of fluid within one or more sections of the fluid management system (e.g., one or more fluid lines) by removing gases therefrom.

Thus, in certain embodiments, the method for degassing may be referred to as a method for forming a fluid column or a method of arranging fluid within a fluid management system. For example, a first fluid, such as saline, from a first fluid source can be driven through a first fluid line into a first fluid source connection of a hemostasis valve coupled to the medical device. A first valve at the first fluid source connection can be closed, resulting in a column of fluid in the first fluid line connection without gas or with a relatively low amount of gas.

Aspiration can then be applied to a sink connection of the hemostasis valve to remove the residual first fluid from the hemostasis valve. This may result in an empty hemostasis valve while a column of fluid is maintained in the first fluid line. The hemostasis valve may then be ready to receive a second fluid, such as contrast media, which may be injected at high pressure. When the second fluid is injected, for example, through a second fluid connection of the hemostasis valve, the column of fluid in the first fluid line may prevent or inhibit the second fluid from flowing into the first fluid line. The second fluid can traverse a path of least resistance, for example, through the lumen of the medical device, instead of through first fluid line.

In certain embodiments, a column or wall of fluid can be formed in a sink line extending from the sink connection to a sink. For example, while aspirating fluid, such as the first fluid, through the sink line via the sink connection, a sink valve at the sink connection can be closed and aspiration can be stopped so that at least some fluid is retained in the sink line instead of flowing to the sink, resulting in a column or wall of fluid in the sink line at the location of the sink connection. In some embodiments, the first fluid may be driven into the hemostasis valve during aspiration in order to form the wall or column of fluid in the sink line. The wall or column of fluid in the sink line may prevent or inhibit a fluid, such as the first fluid or second fluid, from flowing into the sink line. For example, a column or wall of fluid may be formed in both the first fluid line and the sink line, as described herein. The second fluid (e.g., contrast media) can then be injected into the hemostasis valve, and the second fluid can flow through the lumen of the medical device instead of into the first fluid line or the sink line. By preventing undesired flow of fluid into the first fluid line and/or the sink line, fluid waste can be prevented and an amount of fluid flowing to a patient can be known and controlled.

In certain embodiments in which a wall or column of fluid is desired in the sink line, various methods may be employed to prevent or inhibit the retrograde drawing of air through the lumen of the medical device (e.g., a catheter) while building the wall or column of the fluid in the sink line. In certain embodiments, the medical device can be inserted into a patient before aspiration so that blood is drawn through the lumen of the medical device and into the sink line. The column of fluid in the sink line may be formed of blood and/or the first fluid.

In certain embodiments, if the medical device is positioned outside of the body, a tip of the medical device can be placed into a container of fluid, such as saline, which can then be aspirated into the sink line. In other embodiments, the tip of the medical device may be blocked (for example, using a plug) so that air is not aspirated from the distal end while aspirating the first fluid to build a column of fluid in the sink line. In other embodiments, a valve (for example, in a valve manifold as described herein), may be closed to obstruct a connection between the lumen and the hemostasis valve or between the lumen and the sink connection to prevent retrograde air from entering the sink line while building a column of fluid.

In one embodiment, the method includes injecting a first fluid at a low pressure from a first fluid source into a first fluid source connection of a hemostasis valve, closing a first valve at the first fluid source connection, applying vacuum to a sink connection of the hemostasis valve to remove residual first fluid, closing a sink valve at the sink connection, and injecting a second fluid at a high pressure from a second fluid source into a second fluid source connection of the hemostasis valve. The method 550 functions to remove dissolved gases from fluids and fluid lines of the fluid management system. The method is used for catheter and fluid preparation but can additionally or alternatively be used for any suitable applications, clinical or otherwise. The method 550 can be configured and/or adapted to function for any suitable fluid degassing technique.

In some embodiments, instead of injecting first fluid at a low pressure from a first fluid source into a first fluid source connection of a hemostasis valve and closing a first valve at the first fluid source connection, the method may instead evacuate particular ports and/or fluid lines and subsequently inject the first fluid from the first fluid source into a first fluid source connection of the hemostasis valve. In certain embodiments, evacuating particular ports and/or fluid lines prior to fluid injection may provide a negative pressure with may assist subsequent flow of fluid through the fluid management system.

In operation of fluidics system 100, an interventionalist may access fluid management portion 102 and interventional portion 104 to perform a method 550. The method 550 may be a degassing method part of an initial configuration for the fluidics system 100. For example, the method 550 may be performed for all or a portion of a fluid management system of a manually driven medical device, a robotically driven medical device, or a combination thereof.

In some embodiments, the degassing method includes injecting a first fluid from a first fluid source into a first fluid source connection of a hemostasis valve, and closing a first valve at the first fluid source connection. In some embodiments, the degassing method includes injecting the first fluid at a low pressure. Vacuum is applied to a sink connection of the hemostasis valve to remove residual first fluid into the sink. A sink valve is closed and a second fluid is injected from a second fluid source into a second fluid source connection of the hemostatic valve. In some embodiments, the first fluid source connection of the hemostatic valve may not be integrated with the hemostatic valve, but may instead be integrated via a wye adapter to integrate the fluid connection with the hemostatic valve. In other embodiments, the first fluid connection may be otherwise separately integrated with the hub.

In some embodiments, the degassing methods described herein may be a scheduled function that can be accomplished in several ways. In a first example, the degassing function may be accomplished using positive pressure in which the system 10 can inject saline into the fluid port that connects with the catheter lumen. In such an example, the saline may then fill the luminal space in the antegrade direction (i.e., distally toward the catheter tip) and also in the retrograde direction (i.e., through an open proximally situated hemostatic valve). In a second example, the degassing function may be accomplished prior to performing the first example and may include closing the hemostatic valve and applying suction through the fluid port or through a fixture that temporarily connects to the distal end of the catheter. In either example, the distal end of the catheter may be temporarily sealed. After purging the luminal air using suction, the system 10 can close a vacuum valve and then open a saline valve to fill the channel with, possibly degassed, saline. Optionally, the distal tip seal may be removed and the first example may be repeated to complete the degassing function. While a method of degassing is described with respect to FIG. 12, the methods described herein may generally be used to clear a fluid (including gasses, liquids, and/or combinations of gasses and liquids) prior to introduction of another fluid.

As shown in FIG. 12, one embodiment of degassing a fluid management system of a robotically driven medical device includes block 552, which recites injecting a first fluid at a low pressure from a first fluid source into a first fluid source connection of a hemostasis valve. For example, fluid from the second source 210b (e.g., saline) may be injected at a low pressure into a first fluid line of the line branch point 208 that is coupled to RHV 248 and/or one or more catheters associated with RHV 248. In some embodiments, the first fluid source includes heparinized saline.

At block 554, the method 550 includes closing a first valve at the first fluid source connection. For example, the third valve 216c may be closed to stop the first fluid from flowing. In some embodiments, the fluid source connection is a fluid line connected to a fluid source via a ball valve. In some embodiments, the third valve 216c is placed adjacent to or within a manifold, which may function as the first fluid source connection.

At block 556, the method 550 includes applying vacuum (e.g., pump) to a sink connection of the hemostasis valve to remove residual first fluid. For example, a sterile field clot capture container, vacuum chamber, and/or control 202 may be triggered to remove the residual first fluid to the aspiration canister 240 through a connection to the RHV 248, such as the first valve 216a.

At block 558, the method 550 includes closing a sink valve at the sink connection. For example, the first valve 216a may function as a sink valve that may close off fluid flow within fluid lines 222a.

At block 560, the method 550 includes injecting a second fluid at a high pressure from a second fluid source into a second fluid source connection of the hemostasis valve. For example, fluid from the first source 210a (e.g., contrast) may be injected at a high pressure from the first source 210a and into a second fluid line of the line branch point 208 that is coupled to RHV 248 and/or one or more catheters associated with RHV 248. In some embodiments, the second fluid is contrast. In some embodiments, the second fluid source connection may not be integrated directly with the hemostatic valve, but may instead be integrated via a wye adapter to integrate the fluid connection with the hemostatic valve. In other embodiments, the second fluid source connection may be otherwise separately integrated with the hub.

In some embodiments, the method 550 further includes actuating a gasket of the hemostasis valve to a high pressure position before injecting the second fluid or before applying the vacuum. For example, a gasket 458 (FIG. 7A) may be part of RHV 248, 448. The gasket 458 may be actuated to open to a high pressure position before injecting the contrast or before applying the vacuum at the RHV 248, 448.

In some embodiments, the method 550 may further include actuating a gasket of the hemostasis valve to a low pressure position before injecting the first fluid. For example, the gasket 456 (FIG. 6A) may be part of RHV 248, 448. The gasket 456 may be actuated to open to a low pressure position before injecting the saline.

FIG. 13 illustrates a schematic view of an example of a fluidics control system 600 that may be used to electronically control the fluidics systems or components described herein and/or perform the methods described herein. The fluidics control system 600 may be configured to automatically adjust the various manifold valves, pumps, hemostatic valves, hubs, and/or catheters described herein in response to commands input by an operator, such as a physician. In response to command inputs by an operator, the fluidics control system 600 may cause a series of responsive events to automatically occur.

In certain embodiments, the fluidics control system 600 can include one or more processors 602. The one or more processors 602 can be configured to automatically adjust the various manifold valves, pumps, hemostatic valves, hubs, and/or catheters described herein in response to commands input by an operator, for example, using one or more controls of the fluidics control system 600. In certain embodiments, the fluidics control system 600 includes a first control 604a, a second control 604b, and a third control 604c, though any suitable number of controls may be provided to correspond to various functions of the fluidics systems described herein.

For example, in certain embodiments, the first control 604a may be a contrast control that can be operated by a user to initiate the introduction of contrast media into a catheter. The second control 604b may be a saline control that can be operated by a user to initiate the introduction of saline into a catheter. The third control 604c can be a vacuum control configured to initiate the application of vacuum to the catheter. In some embodiments, each unique catheter may have its own unique first control 604a, second control 604b, and/or third control 604c. Alternatively, each of the controls 604a, 604b, 604c (hereinafter “controls 604a-604c”) may be actuated to cause a particular response in a plurality of catheters of the fluidics system.

The processor 602 may receive signals from the controls 604a-604c, and in response, initiate corresponding actions in the components of the fluidics system. For example, the processor 602 may be configured to generate output signals that cause responsive actions to be performed by the components of the fluidics system. For example, in certain embodiments, in response to initiation of the first control 604a by a user, the processor 602 can be configured to open a first contrast valve, close a first saline valve, and close a first vacuum valve associated with a unique catheter. In certain embodiments, the processor 602 may also actuate a first contrast media pump in response to actuation of the first control 604a or a separate unique control.

In certain embodiments, the processor 602 may also adjust a hemostasis valve of the unique catheter to a high compression state as discussed herein in response to actuation of a control, such as the first control 604a or a separate unique control. Although one processor 602 is shown in FIG. 13, in other embodiments, a plurality of processors 602 may be used to control the fluidics systems described herein. For example, each of the controls 604a-604c may communicate with a unique processor.

As described herein, for example with reference to FIG. 1B, in certain embodiments, a catheter system may have a valve manifold in communication with a hub of a catheter. The valve manifold may include a first port configured for connection with a source of vacuum, a second port configured for connection with a source of saline, and a third port configured for connection with a source of contrast media. The fluidics control system 600 (e.g., via the processor 602) can be configured to adjust the valve manifold (for example, in response to operation of one or more controls, such as controls 604a-604c) into an aspiration mode in which the first port is in communication with a lumen of the catheter, and communication between the second port and the lumen and the third port and the lumen is obstructed. In certain embodiments, the fluidics control system 600 (e.g., via the processor 602) can be configured to adjust the valve manifold into a contrast injection mode in which the third port is in communication with the lumen, and communication between the first port and the lumen and between the second port and the lumen is obstructed. The fluidics control system 600 (e.g., via the processor 602) can be configured to control a volume of delivered contrast media.

In certain embodiments, the fluidics control system 600 (e.g., via the processor 602) can be configured to adjust a hemostasis valve of a first catheter between a low sealing force mode or low compression mode and a high sealing force mode or high compression mode. In certain embodiments, the fluidics control system 600 (e.g., via the processor 602) can be configured to adjust the hemostasis valve into the high sealing force mode or high compression mode, and to adjust the valve manifold to selectively place the third port into communication with the lumen while simultaneously blocking the first port and the second port from communicating with the lumen (for example, in response to a human input such as operation of such as operation of one of the controls of the control system).

In certain embodiments, the fluidics control system 600 (e.g., via the processor 602) can be configured to determine a sealing force of the hemostasis valve around a second catheter or a guidewire extending through the hemostasis valve (for example, in response to a human input such as operation of one of the controls of the control system). In certain embodiments, the fluidics control system 600 (e.g., via the processor 602) can be configured to increase the sealing force of the hemostasis valve if the fluidics control system 600 determines that the sealing force of the hemostasis valve around the second catheter or guidewire is low.

In certain embodiments, the processor 602 can be configured to send a first control signal to place the hemostasis valve into the high sealing force mode or high compression mode (for example, in response to human input, such as operation of one of the controls of the control system). In certain embodiments, the processor 602 can be configured to send a second control signal to open the contrast valve (for example, in response to human input, such as operation of one of the controls of the control system). In certain embodiments, the processor 602 can be configured to send a third control signal to place the hemostasis valve into the low sealing force mode or low compression mode (for example, in response to human input, such as operation of one of the controls of the control system).

In certain embodiments, the processor 602 can be configured to send a fourth control signal to a robotic catheter drive system to axially adjust the second catheter with respect to the first catheter (for example, in response to human input, such as operation of one of the controls of the control system). In certain embodiments, the processor 602 can be configured to send a fifth control signal to the robotic catheter drive system to axially proximally withdraw a guidewire from the second catheter prior to opening the contrast valve (for example, in response to human input, such as operation of one of the controls of the control system). One or more of the first control signal, second control signal, third control signal, fourth control signal, or fifth control signal can be sent in response to a single human input. Any of the first control signal, second control signal, third control signal, fourth control signal, or fifth control can be sent in response to a unique human input.

FIGS. 14-63 disclose embodiments of systems, components, and methods for fluidics systems that administer fluids and remove fluids and material (e.g., clots) during medical procedures. These systems and methods also may include a programmable and/or automated (or semi-automated) fluid injection and removal system to perform procedures, and may be coupled to robotically driven interventional devices, manually driven interventional devices, or any combination thereof. A controller in the system can be configured to control the fluid administration equipment of a robotic catheter system to ensure proper diagnostics and/or treatment is provided. Such a controller can include or more hardware processors that are capable of executing instructions for controlling fluid administration equipment (e.g., actuating valves and pumps). When used herein, a “controller” can refer to one controller or one or more controllers. In referring to a controller or controllers, for ease of reference, a “controller” or “one or more controllers” may be referred to herein simply as “a controller” or “the controller” unless otherwise indicated by context or explicitly, and accordingly reference to “a controller” or “the controller” should be understood to include disclosing one controller or one or more controllers.

Disclosed embodiments include systems of a robotic catheter system for managing fluidics. As described further below (e.g., in reference to FIG. 14), in some embodiments the system can include a pump station and a cassette. The cassette can include components and fluid communication channels of a saline subsystem, a contrast subsystem, and a vacuum subsystem. For example, cassette can include connections/ports to connect to a tubing set and sources of saline, contrast, and vacuum, one or more peristaltic pumps or interfaces with one or more peristaltic pumps, one or more valves, one or more sensors, one or more contrast pumps, one or more vacuum canisters or interfaces to one or more vacuum cannisters, one or more clot pods, one or more drip chambers, and/or fluid communication channels for communicating saline, contrast, and vacuum from the saline, contrast, and vacuum sources to the hubs and catheters. The pump station can include components that interact with the subsystems in the cassette when the cassette is coupled to the pump station. In an example, the pump station can include one or more valve actuators, one or more peristaltic pump drive mechanisms, one or more contrast pump drive mechanisms, configured to actuate and drive valves and pumps in the cassette that are part of the saline, contrast, and vacuum subsystems. The pump station can also include one or more sensors that are configured to sense fluid flowing in certain fluid communication channels of the cassette.

In some embodiments, the cassette and the pump station include corresponding electrical contacts or connections (both referred to as “contacts”) that are connected when the cassette is coupled to the pump station to connect electrical components (e.g., sensors) in the cassette to the pump station. The electrical contacts may include contacts for providing information from a component in the cassette (or the hub coupled to the cassette) to a controller in the pump station or a controller in communication with the pump station that is configured to operate the fluidics system, or another system that utilizes such information. The electrical contacts may include contacts for providing power to a component in the cassette. In some embodiments, the electrical contacts can also electrically connect the pump station to one or more hubs via the cassette. Accordingly, the electrical contacts can provide power to a component in one or more hubs via the cassette (and via electrical connections between the cassette and the one or more hubs). Certain components of the system can be configured to be disposable and certain components can be configured to be reusable. For example, the one or more hubs and catheters coupled to the hubs, a tubing set, and/or cassette can be configured to be disposable. Valves related to controlling providing fluids and providing vacuum in system can be referred to collectively as a “valve assembly” for ease of reference. The valve assembly can include, but is not limited to, valves in a saline subsystem, contrast subsystem, and vacuum subsystem that are located in a pump station, cassette, and/or the one or more hubs. As described in examples below, the system provides saline, contrast, and vacuum, from saline, contrast, and vacuum sources (respectively) through fluid communication channels to the one or more hubs and catheters coupled to the hubs. The fluid communication channels can include channels, tubes, ports, lines connectors, and other structure to communicate fluid and provide vacuum. Unless otherwise indicated explicitly or by context “channels,” “tubes,” “lines,” molded apertures and structures, and other structures or portions of components through which a fluid or gas flows may be used synonymously herein as referring to a fluid communication channel, or simply a “channel” for ease of reference. For example, the fluid communication channels can include channels in the cassette, one or more tubes that are part of tubing set, and tubes and/or channels located in a hub, and the fluid communication channels can be collectively referred to as a fluid communication system.

Embodiments of a saline subsystem, a contrast subsystem, and a vacuum subsystem can be used to perform methods for controlling fluid administration equipment. Certain components of a robotic catheter system that are part of, or related to, a fluidics system, are actuatable by a controller, or provide information to a controller. The controller is configured to perform the methods for controlling fluid administration equipment by actuating such components, based on predetermined instructions, user input, and/or sensed information from the system. and As illustrated in FIG. 14, a controller can be remotely located to control the robotic catheter system from any location. Priming the saline subsystem, priming the contrast subsystem, injecting contrast, aspirating a clot, and back-bleeding a hemostasis valve are examples of some if the processes that can be controlled at least in part by a controller. Valves, pumps and other actuatable (or movable) components can be driven to be in a certain state or position by a controller. In some embodiments, the state or position of actuatable components can be determined by sensing mechanism on the component such that the controller can determine the current position of the component before, during, or after a process. In some examples, the sensing mechanism can include a switch, encoder, and the like. In some embodiments, the current state or position of the components can be electronically stored information (e.g., in a table or file) and the controller is configured to determine the position of one or more components by accessing the stored information. In an example, before contrast is injected using a selected catheter in a system having multiple catheters, a valve coupling the selected catheter to a contrast subsystem can be determined to be aligned in an open position to provide contrast to the selected catheter, and one or more valves coupling non-selected catheters to the contrast subsystem can be determined to be aligned in a closed position to de-couple the non-selected catheters from the contrast subsystem. Such determinations may be made using the stored information or by using information from a sensing mechanism. The processes described herein are examples of some of the processes a controller can be configured to perform by controlling fluidic equipment, either automatically or semi-automatically, based on one or more user inputs, sensed information, and/other information.

The disclosed fluidics systems are configured to provide saline, contrast, and vacuum to one or more hubs. In an example, the one or more hubs may comprise a first hub, a second hub, and a third hub. A catheter is coupled to each of the one or more hubs, and each of the hubs is configured to provide saline, contrast, and vacuum from the fluidic system to the lumen of the catheter coupled to the hub. For simplicity of disclosure, unless otherwise specified indicated by context, providing saline, contrast, or vacuum to a hub also refers to providing saline, contrast, or vacuum to the catheter coupled to the hub. The multi-channel fluidics systems is configured to provide separately controllable amounts of saline, contrast, and vacuum to each of the one or more hubs. Being able to separately control the saline, contrast, and vacuum to each hub is advantageous because each catheter has a different sized lumen due to the catheter having a different inside diameter, and the amounts of saline, contrast, and/or vacuum needed during their use may be different based on the lumen size. Each catheter can also have a different effective lumen size which may change at certain times during the procedure based on its lumen containing another catheter or guidewire nested inside its lumen. Being able to separately control the saline, contrast, and vacuum to each hub can also be advantageous because during a medical procedure different amounts of saline, contrast, or vacuum at each catheter may be desired based on what a particular catheter is being used for. In other embodiments, such a fluidics system can be configured as a one-channel fluidics system that provides controlled amounts of saline, contrast, and vacuum to one hub, or a multi-channel fluidics system configured to provide controlled amounts of saline, contrast, and vacuum to two hubs, or four or more hubs.

In various embodiments, the fluidics systems can have similar or the same features as other fluidic systems described herein. In an example, a fluidics system includes a pump station and a cassette which is releasably couplable to the pump station. The pump station can include components that may be capital equipment. The cassette can include components that may be designed for a one-time use and the cassette is disposable. In an example of use, the cassette is a sterilized disposable component that is coupled to the pump station prior to performing a procedure, and the cassette is removed and disposed of after the procedure is completed. The fluidic system can include numerous valves that control saline, contrast, or vacuum, located in a cassette, pump station, or hub (as illustrated in and can be referred to collectively as a valve assembly. In various embodiments, portions of the valve assembly can be located in the pump station or in the cassette. Various embodiments of the valve assembly can include additional valves, fewer valves, or different valves, than illustrated in the figures. The fluidic system can also include sensors in the saline, contrast, and vacuum subsystem, and these senses can be collectively referred to as a sensor assembly. In various embodiments, portions of the sensor assembly can be located in the pump station or in the cassette. Various embodiments of the sensor assembly can include additional sensors, fewer sensors, or different sensors, than illustrated in the figures.

Each of the hubs may include a plurality of components related to providing saline, contrast, and vacuum to a catheter, and components related to moving the hub and an interventional device attached thereto in an axial direction and rotating the catheter. The tubing set can also include electrical connections to communicate control information to the one or more hubs and/or receive information from components of the hubs.

Fluidic-related processes performed by the robotic catheter system are controlled at least in part by a controller and can be controlled based on a user input, predetermined programmed processes, parameter information, and/or based on conditions sensed by the system. The embodiments described herein provide examples of configurations of a fluidics system that can be used in a remotely controllable robotic catheter system to perform fluidic-related processes for a medical procedure, for example, priming a saline subsystem, priming a contrast subsystem, operating a hemostasis valve, injecting contrast, aspirating a clot, and back-bleeding. To perform such processes valves, pumps, and other actuatable (or movable) components can be driven to be in a certain state or position by a controller. In some embodiments, the state or position of actuatable components can be determined by a position sensing mechanism on the component such that the controller can determine the current position of the component before, during, or after a process. In some embodiments, the positioning sensing mechanism can include a switch, encoder, etc. The sensing mechanism can be incorporated in the cassette or the mount to determine a position of a movable component (e.g., valve, pump) or on an actuator that drives the movable component. In some embodiments, a current state or position of the components can be electronically stored (e.g., in a table or file) and the controller is configured to determine the position of one or more components by accessing the stored information. In an example, before contrast is injected using a selected catheter in a system having multiple catheters, a valve coupling a selected catheter to a contrast subsystem can be determined to be aligned in an open position to provide a flow-path for contrast to the selected catheter, and one or more valves coupling non-selected catheters to the contrast subsystem can be determined to be aligned in a closed position to shutoff a flow-path of contrast to non-selected catheters. Such determinations may be made using the stored information and/or by using information from a sensing mechanism. The processes described herein are some of the processes a controller can be configured to perform by controlling fluidic equipment, either automatically or semi-automatically, based on one or more user inputs, sensed information, and/other information. An example of processes that can be partially or completely controlled by a controller using embodiments described herein include, but are not limited to:
providing a continuous “low” saline flow to each of one or more catheters, for example, at about 1 mL/minute of saline;
providing a “high” saline flow to flush a single one of the one or more catheters during a procedure, for example, at about 6 mL/second;
providing a contrast injection in any selected catheter of the system (for example, in any of three catheters of the system), the contrast injection provided with minimum delay after receiving a signal from a medical practitioner to inject contrast and being at, for example, up to about 4 mL/second at up to about 400 psi or about 500 psi;
determining catheter patency, of any of the one or more catheters at a medical practitioner's request;
providing aspiration at high (e.g., full) vacuum on one or more catheters;
back-bleeding a selected one of the one or more catheters with low vacuum;
priming the saline subsystem without user interaction;
priming the contrast subsystem without user interaction;
priming fluid communication channels between the cassette and the plurality of mounts
priming the saline flow-path in the plurality of mounts
priming the contrast flow-path in the plurality of mounts
sensing air bubbles in the saline subsystem and/or a mount and performing corrective actions;
sensing air bubbles in the contrast subsystem and/or a mount and performing corrective actions; and
performing actions related to refills of fluids (e.g., contrast, saline).

FIG. 14 illustrates an example of a robotic catheter system that includes a remotely located system (“remote system”) and a locally located system (which may be referred to a “local system,” “bedside system,” or a “near-patient system”). FIG. 14 also illustrates an example of certain components of the local system that includes components of a fluidic management system (“fluidics system”) including actuatable components that are moved by a controller and sensors that provide information to a controller that is configured to control the fluidics systems and other aspects of the robotic catheter system based on the information, user inputs, and predetermined information, according to some embodiments.

In some embodiments, the robotic catheter system includes a local portion including a system controller 2220 at the bedside location (where the patient is located), and a remote portion including a control system 2210 located remotely from the patient. “Local” or “bedside” or “local location” as used herein are broad terms that refer to the location where the “local” portion of the robotic catheter system is located, that is, at the actual location where the patient is receiving medical treatment using the robotic catheter system. “Remote” or “remote location” or “remote portion” as used herein is a broad term and generally refers to a location other than where the patient/local system is located when the patient undergoes a medical procedure using the robotic catheter system. In some circumstances, the remote location is within the same building as the patient. For example, in a different portion of a room where the patient is, e.g., on the other side of a barrier or positioned bedside, in the same building but in a room next door to the patient, in the same building but in a different room from the patient, in a different building from the patient, or in a different town or city or state. In some embodiments the remote portion is located several feet away, or hundreds or thousands of miles away from the local portion. The control system 2210 includes one or more displays 1309 for displaying information relating to the robotic catheter system, the medical procedure, and/or the patient. For example, the control system 2210 can receive information from the system controller 2220 relating to an aspiration procedure, a back-bleeding procedure, and the like, and display the information on the one or more displays 1309. The information can include, for example, images depicting a drip chamber and/or a clot pod of a fluidics system located at the bedside location, patient information (e.g., patient vitals), system information including images and/or data about the interventional devices (e.g., position or location information), information about processes occurring on the robotic catheter system, operating room information, and the like. The images can be, for example, video generated and communicated in real-time (or near-real-time) relating to a medical procedure being performed that the medical practitioner can view and use to determine action(s) to be taken. For example, the images can depict the volume of fluid in a drip chamber, material that is collected in a clot pod, and/or fluidic lines/channels), and the like. The control system 2210 also include a user interface 1313. The user interface 1313 can include multiple interfaces. For example, one or more displays, and/or one or more user input devices, for example, switches, buttons, joysticks, touchscreen controls, a handheld controller, etc. The robotic catheter system is configured such that a medical practitioner at the remote location can view information from the local controller 2230 on displays 1309 of the remote controller 2210, and provide one or more signals, via the interface 1313, to the local system controller 2220 for controlling actions of the robotic system during a medical procedure.

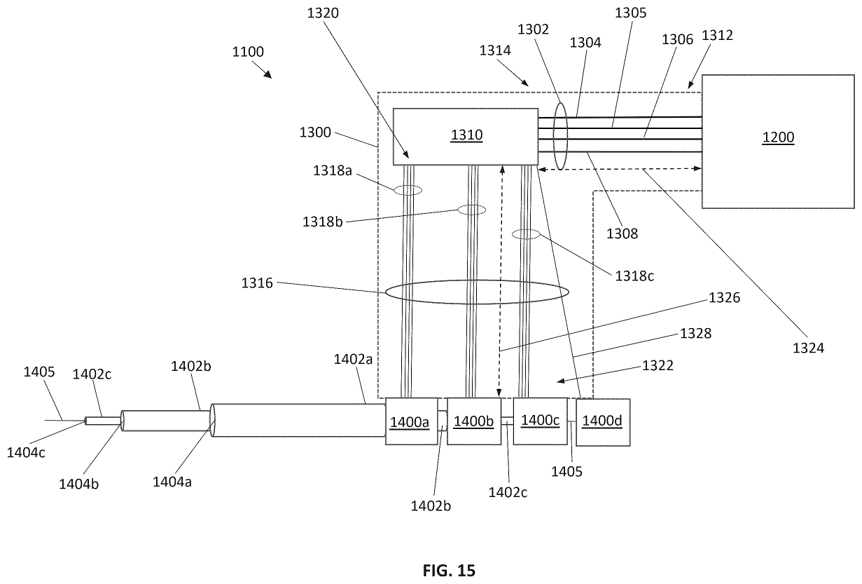

The remotely located control system 2210 is configured to be in communication with a system controller 2220 which is part of the local portion of the system. The system controller 2220 may include multiple controllers each having one or more processors. In this example, the system controller 2220 includes an interface 2235. The interface 2235 can include multiple interfaces and may be a user interface. The interface 2235 can be configured for communicating with the remotely located control system 2210. For example, the interface 2235 can be configured to receive control signals from the control system 2210 and communicate corresponding signals to a controller (e.g., controller 2230 or pump station 2240) to perform a fluidic action (e.g., inject contrast from a catheter, provide aspiration from a catheter), or to move one or more of the hubs (1400, FIG. 15) axially to correspondingly move an attached elongated devices (e.g., a catheter, a guidewire) axially, or to cause one or more hubs to move an attached elongated device rotationally around its longitudinal axis. The interface 2235 can also be configured to receive control information from a user located locally (e.g., for a fluidic action or to control movement of the hubs and coupled to interventional devices, and to provide information (status, images, etc.) to a local user. The interface 2235 can include multiple interfaces. For example, one or more displays and displaying information relating to the robotic catheter system, the medical procedure, and/or the patient. The interface 2235 can also include one or more user input devices, for example, switches, buttons, touchscreen controls, etc.

The interface 2235 can also be configured to communicate information to the remotely located control system 2210. The communicated information can be related to a received control action, fluidic information, catheter position information, status information, images or video, audio and other communications from the robotic system or users located locally with the patient, and any other information that may be needed to control the robotic catheter system from the control system 2210. The interface 2235 can also be configured to receive inputs from users of the robotic catheter system located with the patient.

The system controller 2220 can include pump station 2240 for performing fluidic-related actions, and a controller 2230 configured for processing user inputs received locally or from the control system 2210, and processing sensor information, and controlling the pump station 2240 and other portion of the robotic control system to perform medical procedures based on the user inputs and the sensed information, including providing saline, contrast, and vacuum to the hubs. In some embodiments, the system controller 2220 can also control providing saline to a femoral sheath.

The robotic catheter system can include actuatable components of a fluidics management system that are movable, as controlled by a controller, to align fluid communication channels to provide saline, contrast, and vacuum from a saline, contrast, and vacuum source (respectively) to hubs, and including certain sensors that are configured to sense a condition related to providing provide saline, contrast, and vacuum to the hubs and provide the sensed information to a controller. The controller can be configured to use the sensed information, user inputs, and/or stored information to control actions of the fluidics system to perform a medical procedure. In various embodiments, a pump station may include a controller to control actuators of the pump station to perform fluidic-related actions (e.g., provide saline, contrast, and vacuum to the hubs). In other embodiments, controller 2230 and/or system controller 2220 can be configured to control actuators and other components of the pump station 2240 to perform fluidic-related actions.

The robotic catheter system illustrated in FIG. 14 can include actuatable components of a fluidics management system that are movable, as controlled by a controller, to align fluid communication channels to provide saline, contrast, and vacuum from a saline, contrast, and vacuum source (respectively) to hub assemblies 1400a-1400c. As indicated above, in some examples the hub assemblies 1400 can include a hub (first subassembly) and a mount (second subassembly). For example, FIG. 19 illustrates an embodiment of three hub assemblies, where a first hub assembly includes hub 724a and a mount 1400a, a second hub assembly includes hub 724b and mount 1400b, and a third hub assembly includes a hub 724c and a mount 1400c. Each of hub 724a-c is structured to be releasably coupleable, mechanically and fluidically, to its respective mount 1400a, 1400b, 1400. Each of the hubs 724a, 724b, 724c has a catheter coupled thereto (catheters 1402a, 1402b, 1402c, respectively). Although some of the examples described herein refer to mounts and components of mounts, this description is not limited to embodiments of hub assemblies that include a hub and a mount, rather the components and processes disclosed can also be implemented in both hub assemblies that do not include a first subassembly and a second hub assembly but instead are housed as a one-part hub assembly. Accordingly, reference to a mount(s) should be understood as also referring to either a multi-part hub assembly (e.g., two-part hub assembly having a hub and a mount) or part of a one-part hub assembly and unless specifically indicated or indicated by context. Correspondingly, reference to a hub assembly should be understood as referring to either a multi-part hub assembly (e.g., two-part hub assembly having a hub and a mount) or a one-part hub assembly.

The fluidics system of the robotic catheter system can include certain sensors that are configured to sense a condition related to providing provide saline, contrast, and vacuum to the mounts 1400a-1400c and provide the sensed information to a controller. For example, the position of a valve, position of the plunger of the contrast pump, information from a pressure sensor, and/or information from an air bubble sensor, etc. The controller can be configured to use the sensed information, user inputs, and/or stored information to control actions of the fluidics system to perform a desired medical procedure. In various embodiments, a pump station 2240 may include a controller to control actuators of the pump station 2240 to perform fluidic-related actions (e.g., provide saline, contrast, and vacuum to the mounts 1400a-1400c). In other embodiments, controller 2230 and/or system controller 2220 can be configured to control actuators and other components of the pump station 2240 to perform fluidic-related actions. Certain components and systems of the robotic catheter system may not be illustrated in FIG. 14 for clarity of this illustration (for example, a hub axial drive system, check valves, a catheter rotational system, controllers of the remote control system 2210, etc.).

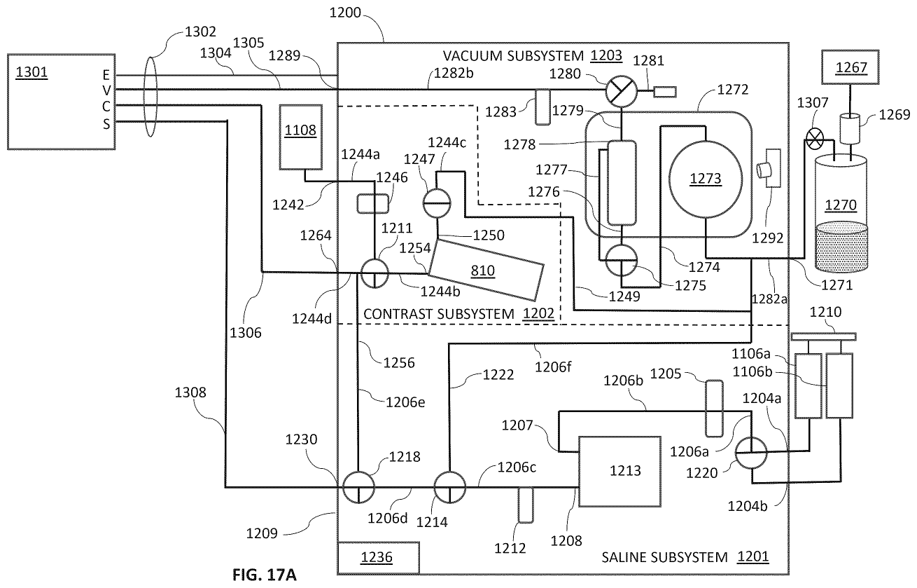

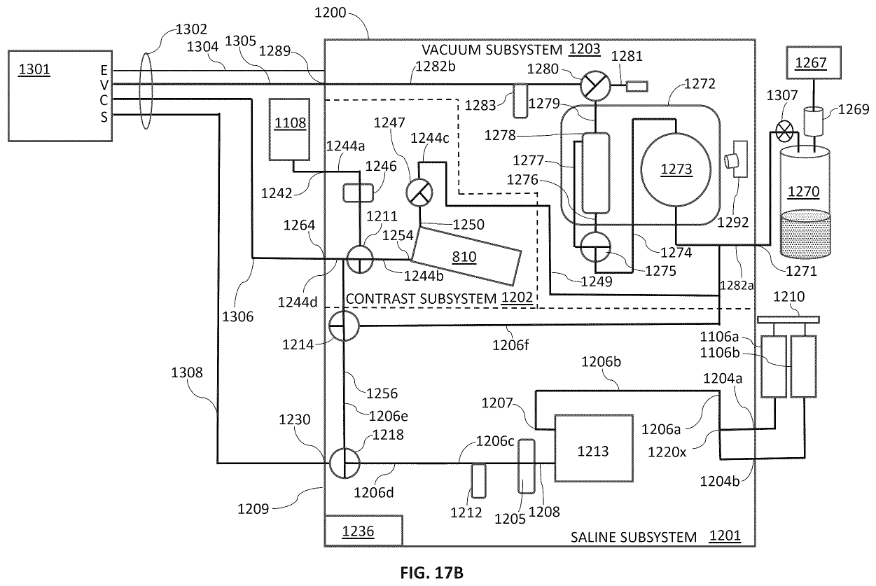

In the embodiment illustrated in FIG. 14, robotic catheter system includes a fluidics assembly 1100 that includes a cassette 1200 which is configured to be releasably attached to a pump station 2240. The cassette 1200 includes a housing 1209 that supports a saline subsystem 1201, a contrast subsystem 1202, and a vacuum subsystem 1203. The saline subsystem 1201, contrast subsystem 1202, and vacuum subsystem 1203 can be all or partially enclosed within the housing 1209. Portions of the saline subsystem 1201, contrast subsystem 1202, and vacuum subsystem 1203 that interface with the components on the pump station 2240 can be positioned on the surface of the housing 1209 or otherwise be accessible to interface with a corresponding component on the pump station. For example, an interface for an actuator to mechanically couple to a valve in the cassette, an interface for a portion of a contrast pump or saline pump in the cassette to mechanically couple to a portion of a contrast or saline pump in the pump station 2240 (e.g., a pump driver). The cassette 1200 can be placed on the pump station 2240 in position 1200a such that components of the cassette 1200 align with corresponding components of the pump station 2240. In an example, to align valves in the cassette 1200 with valve actuators of the pump station 2240, to align a portion of a peristaltic pump in the cassette 1200 (e.g., a saline tube) with a portion of a peristaltic pump in the pump station 2240, and/or to align a contrast pump in the cassette 1200 with a contrast pump drive mechanism in the pump station 2240. In another example, an electrical interface 1236 having a plurality of electrical connections on the cassette 1200 can be aligned with a corresponding electrical interface 1112 on the pump station 2240. The saline subsystem 1201, contrast subsystem 1202, and vacuum subsystem 1203 are further described in reference to FIGS. 17A and 17B. The cassette 1200 also may include an electrical interface 1236 which can be coupled to a pump station electrical interface 1112 using various corresponding mechanical-electrical connectors can be used. For example, using a socket and a plurality of extended connectors built into the cassette and the pump station, using an electrical pigtail on one or both of the cassette and the pump station, using corresponding pogo pins connectors, etc. In operation, after the cassette 1200 is coupled to the pump station 2240, one or more saline source 1106 can be coupled to the saline subsystem 1201, one or more contrast source 1108 can be coupled to the contrast subsystem 1202, and one or more vacuum source 1110 is coupled to the vacuum subsystem 1203.

The fluidics assembly 1100 also can include one or more mounts 1400a-c, and communication channels 1300 coupled to the cassette 1200 and to each of the mounts 1400a-c, the communication channels 1300 structured to separately communicate saline, contrast, and vacuum from the cassette 1200 to the mounts 1400. As indicated above, the terms “channels,” “tubes,” “lines,” molded apertures and structures, valves, and other structures or portions of components through which a fluid or a gas flows may be referred to herein generally as a fluid communication channel, or a “communication channel,” or a “channel.” The vacuum communication channel provides a path for aspirating material from any of the catheters 1402a-c coupled to the mounts 1400a-c, respectively, to a clot pod or vacuum canister (for example, as illustrated in FIGS. 17A, 17B). In the embodiment illustrated in FIG. 14, a guide catheter 1402a is coupled to a guide catheter mount 1400a, a procedure catheter 1402b is coupled to a procedure catheter mount 1400b, and an access catheter (or insert catheter) 1402c is coupled to an access catheter mount 1400c, the catheters forming a concentric stack that are individually movable, and to which saline, contrast, and vacuum can be individually provided to, simultaneously or individually to one or more of the catheters. For example, in some embodiments, vacuum can be provided to one or more of the catheters (e.g., the procedure catheter 1402b, the guide catheter 1402a) to perform an aspiration procedure (e.g., aspirate a clot). In some embodiments, aspiration may be performed through two catheters (e.g., the procedure catheter 1402b and the guide catheter 1402a) simultaneously. For example, during a thrombectomy procedure, a clot may become engaged with or corked at a distal end of the procedure catheter 1402b (or another inner catheter). In such cases, it may be necessary to remove the procedure catheter 1402b (or other inner catheter) from the vasculature of the patient to remove the clot. As the procedure catheter 1402b (or other inner catheter) is retracted from the guide catheter 1402a (or other outer catheter), debris from the stuck clot can dislodge. In such embodiments, application of vacuum at both the procedure catheter 1402b (or other inner catheter) and the guide catheter 1402a (or other outer catheter) can beneficially prevent the debris from flowing distally into the vasculature of the patient by aspirating the debris and thus reducing the risk of embolization.

In some embodiments, the clot may become engaged with or corked at the distal end of the guide catheter 1402a (or other outer catheter). Vacuum through the guide catheter 1402a (or other outer catheter) may prevent dislodgement of the clot from the guide catheter 1402a (or other outer catheter). In some instances, it may not be readily apparent if portions of a clot are engaged with the guide catheter 1402a (or other outer catheter) or the procedure catheter 1402b (or other inner catheter). In such instances, vacuum through both the guide catheter 1402a (or other outer catheter) and the procedure catheter 1402b (or other inner catheter) may prevent debris from flowing distally into the vasculature of the patient by aspirating the debris and thus reducing the risk of embolization.

In the example of FIG. 14, the mounts 1400a-c are part of two-part hubs. The catheters 1402a-c are attached hubs 724a-c that mechanically couple to mounts 1400a-c, respectively. Hubs 724a-c are also configured to fluidically couple to mounts 1400a-c, respectively, for example, via a connector (port) 1434a-c (FIG. 19). The mounts 1400 are controllable to move in the same axial direction and the catheters are sized and configured to be positioned in a nested (concentric) configuration of the interventional devices, that is, the access catheter 1402c can be positioned in part of, or in the entirety of, the lumen of the procedure catheter 1402b, and both the access catheter 1402c and the procedure catheter 1402b can be positioned in part of, or in the entirety of, the lumen of the guide catheter 1402a, based on the positioning of the mounts 1400. A guide wire (e.g., 1405, FIG. 15, coupled to mount 1400d) can also be a part of the concentric stack of interventional devices.

In some embodiments, all or part of the fluidics system that is attached to the pump station is made to be disposable. In some embodiments, part of the fluidics system that is attached to the pump station is made recyclable. In either case, the system fluidics system can be advantageously designed to be as lowest cost as feasible. For example, valves in the fluidics system can be positioned in the cassette 1200 while their corresponding valve actuators can be positioned in the pump station. The contrast pump can be positioned in the cassette and the corresponding contrast pump driver can be positioned in the pump station. A clot pod can be positioned in the cassette and a vacuum pump and a vacuum regulator can be positioned external to the cassette, as illustrated in FIG. 17A. In some embodiments, a camera 1292 (FIG. 17A) can be used to generate an image of a drip pod 1272 that includes a drip chamber 1278 and a clot pod 1273 (FIG. 17A) for viewing on a local or remotely located display at the remote controller system 2210, where the camera 1292 (capital equipment) can be positioned in the pump station 2240 or somewhere bedside (locally) and the drip pod 1272 (disposable) can be positioned in the cassette. As imaging technology continues to become less expensive, in other embodiments a relatively inexpensive camera or sensor can be positioned in the cassette to generate an image of the drip chamber 1278 and/or the clot pod 1273.

FIG. 15 is a schematic of the example of the fluidics assembly 1100 of FIG. 14, illustrating some components in further detail. In FIG. 15, the fluidics assembly 1100 includes a cassette 1200 that couples to the pump station and to a saline source, a contrast source, and a vacuum source, for example as described in reference to FIG. 14 and further in reference to FIG. 17A. The fluidics assembly 1100 also includes a plurality of fluid communication channels 1300 to provide saline, contrast, and vacuum to one or more mounts 1400a-c and to the hubs 724a-c (FIG. 19) and catheters 1402a-c coupled to the mounts 1400a-c. The fluid communication channels 1300 also can include a plurality of electrical leads that connect components in the mounts (e.g., bubble sensors, pressure sensors, etc.) to the electrical interface 1236 of the cassette to provide signals to the control system 2220 when the cassette 1200 is coupled to the pump station 2240 (FIG. 14). In systems that include a plurality of hub assemblies, different hub assemblies may be configured differently or may have different fluids communicated to them, which are provided to a catheter. For example, in a system with a plurality of hub assemblies, one hub assembly may only provide saline to a catheter, and/or one hub assembly may provide saline and contrast to a catheter, and/or one hub assembly may provide saline and vacuum to a catheter, and/or one hub assembly may provide saline, contrast, and vacuum to a catheter.

One challenge of a fluidics system that provides fluids (e.g., saline and contrast) that enter a patient via a catheter is to provide such fluids in air-free (that is, bubble-free) fluid flows. As described in detail above and further below, typically when the cassette, fluid communication channels, hubs/mounts, and catheters are provided for use in an operating room they are not filled with fluid, and thus require priming to remove air from the saline and contrast flow-paths to prevent air bubbles entering a patient during a medical procedure. Once primed, air bubble detectors in the cassette and the mounts (e.g., illustrated in FIGS. 17A and 19) can help mitigate risks associated with air bubbles within saline and contrast flow-paths. However, because of the dangerous consequences of air entering a patient's bloodstream, where it is possible the fluid communication channels are visually inspected for air bubbles by a medical partitioner after the saline and contrast flow-paths are primed. Because of flexibility and mobility requirements for providing fluids from static saline and contrast fluid sources across a certain distance to moving mounts/catheters, at least a portion of the fluid communication channels between the cassette and the mounts can include flexible clear tubing. During the visual inspection, each centimeter of all the clear tubing supplying saline or contrast from the cassette 1300 to the mounts 1400 is carefully inspected to determine if air bubbles are present, and if so the lines can be re-primed. The length of time it takes to inspect the clear tubing for air bubbles directly relates to the total length of the contrast and saline tubing between the cassette and the mounts. In some examples (e.g., FIG. 14), the pump station is located several feet away from the set of mounts 1400 to provide room around the patient, and the saline and contrast fluid communication channels 1300 between the cassette 1200 and mounts 1400 may be about 14 feet long. If, as illustrated in FIG. 15, there are three mounts 1400a-c coupled to the communication channels 1300 and the communication channels include individual tubes for saline and contrast for each of three mounts 1400, this results in 84 linear feet of saline and contrast tubing to inspect. To make inspection of the tubing easier, the communication channels can be configured in a “flat” ribbon-like design with the saline and contrast communication channels next to each other such that the contrast and saline tubing can be inspected at the same time, which effectively reduces the length of tubing to be inspected to 42 feet (14′ per mount). During the inspection, as the lines are each handled from end-to-end the medical practitioner typically will change gloves for each line (e.g., three glove changes). Such an inspection can take between 7 and 10 minutes, which is a long delay before proceeding with the operation when every minute of delay of performing a thrombectomy can result in further harm to the patient.

The first tubing set 1302 includes a one saline tube (channel) 1308 in fluid communication with a first saline flow-path in the cassette 1200 and a second saline flow-path in the splitter 1310, one contrast tube (channel) 1306 in fluid communication with a first contrast flow-path in the cassette 1200 and a contrast flow-path in the splitter 1310, and one vacuum tube (channel) 1305 in fluid communication with a first vacuum flow-path in the cassette 1200 and a second vacuum flow-path in the splitter 1310. An example first saline flow-path, first contrast flow-path, and first vacuum flow-path, in the cassette, are illustrated by the saline subsystem 1201, the contrast subsystem 1202, and the vacuum subsystem 1203 in FIG. 17A. The splitter 1310 provides saline, contrast, and vacuum from the first tubing set 1302 to the second tubing set 1316, and includes channels forming a second saline flow-path, a second contrast flow-path, and a second vacuum flow-path, where the second saline flow-path, the second contrast flow-path, and the second vacuum flow-path each having a plurality of branches structured to provide outputs of saline, contrast, and vacuum to each mount 1400 from the single inputs of saline, contrast, and vacuum from the first tubing set 1302. The first tubing set 1302 can be configured in a ribbon-like design. In some embodiments, the length 1324 of the first tubing set can be between about 5 feet and about 17 feet in length based on the specific embodiment. In some examples, the ratio of the first length to the second length is greater than about 1:2 (e.g., about 1:3, 1:4, 1:5, 1:6, of 1:7) to minimize the amount of tubing that needs to be visually inspected. In a specific embodiment, the length of the first tubing set is about 12 feet in length. The tubes in the first tubing set have inner diameters to suitably provide saline, contrast and vacuum to the catheters. As a nonlimiting examples, the inner diameter of saline and contrast tubes (channels) in the first tubing set and the second tubing set can be about 0.071 inches. As a nonlimiting example, the inner diameter of vacuum tubes in the first tubing set and the second tubing set can be about 0.110 inches. The first tubing set 1302 can also include an electrical channel 1304. The electrical channel includes multiple electrical leads to communicate electrical signals between the mounts 1400 and the cassette 1200, and can be coupled to the electrical interface 1236. Sensors and electrical components in the cassette 1200 can also be coupled to the electrical interface 1236.

The second tubing set 1316 includes one or more tube groups 1318, each tube subgroup configured to provide a mount 1400 with saline, contrast, and vacuum. Each tube group 1318 can also provide each mount 1400 with one or more electrical leads. In the example in FIG. 15, each tube group 1318a-c includes a proximal end 1320 coupled to the splitter 1310 and a distal end 1322 coupled to one of the one or more mounts 1400a-c. Each tube group 1318a-c includes a saline subchannel in communication with the second saline flow-path in the splitter 1310, a contrast subchannel in communication with the second contrast flow-path in the splitter 1310, and a vacuum subchannel in communication with the second vacuum flow-path in the splitter 1310. Each tube group 1318 can be configured in a ribbon-like design. In some embodiments, the length 1326 of the second tubing set can be between about 1 foot and about 4 feet. In a specific embodiment, the length 1326 of the second tubing set is about 2 feet. In this embodiment, the length of tubing in the first tubing set and the second tubing set needed to be inspected is significantly reduced from 42 feet to 18 feet, which correspondingly drastically reduces the inspection time to about 3-4.5 minutes, advantageously reduces set-up time such that the medical procedure (e.g., a thrombectomy) can begin much sooner, which increases the patient's likelihood of a more successful recovery. In addition, this significant reduction in the amount of tubing to inspect also minimizes glove changes by the medical practitioner inspecting the tubing set from three glove changes to one glove change. The second tubing set 1316 can also provide one or more electrical leads 1328 to another mount that does not require fluids, for example, a guide wire mount 1400d configured to axially move and rotate a guide wire 1405 coupled to the guide wire mount 1400d, the guide wire 1405 configured to be positioned partially or fully in the lumen of access catheter 1402c. In some embodiments, connectors may be used to couple the second tubing set 1306 to the cassette 1200 and/or the splitter 1310. However, use of connectors in fluidic systems can cause air bubbles to be formed and/or trapped in a portion of the connector. In this embodiment, advantageously connectors are not used, rather the second tubing set 1316 is coupled to the splitter 1310 and the mounts 1400 such that the second tubing set 1316 is not meant to be decoupled prior to or during a medical procedure (for example, permanently, semi-permanently).

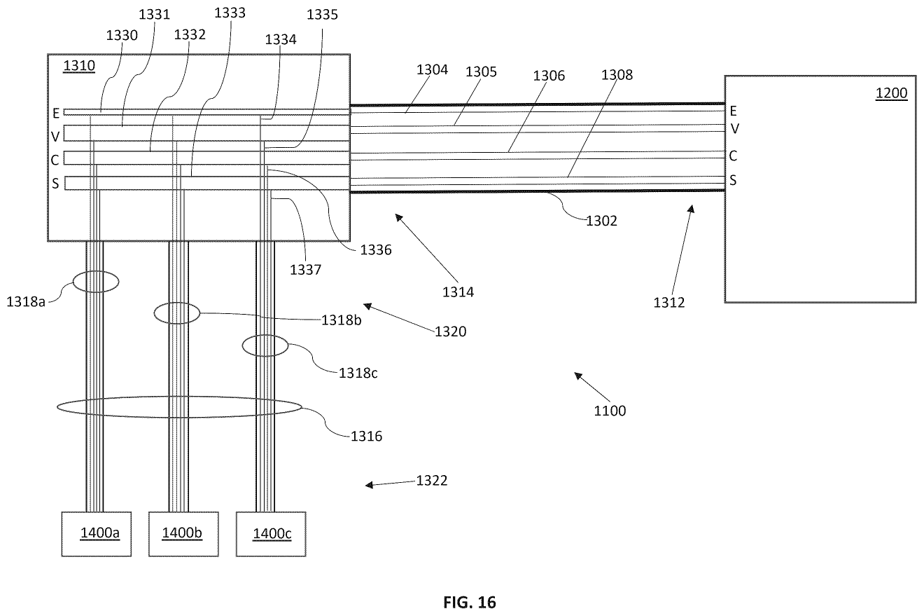

FIG. 16 is a schematic of an example of the fluidics assembly 1100 illustrated in FIG. 15 illustrating some additional features. The fluidics assembly 1100 includes cassette 1200, splitter 1310, and one or more mounts 1400, first tubing set 1302 coupled to the cassette 1200 and splitter 1310, and second tubing set 1316 coupled between the splitter 1310 and the one or more mounts 1400, the first tubing set 1316 having a saline channel 1308, a contrast channel 1306, and a vacuum channel 1305 for providing saline, contrast, and vacuum to the splitter 1310. In this example, the fluidics assembly 1100 is shown having three mounts 1400a-c that receive saline, contrast, and vacuum from the cassette 1200. The first tubing set 1302 also includes an electrical channel 1304 having a plurality of electrical leads for connecting electrical components in the mounts 1400 to an electrical interface of the cassette 1200. The second tubing set 1316 includes tube groups 1318a-c providing saline, contrast, and vacuum to mounts 1400a-c. Each of the tube groups 1318ac includes a saline subchannel 1333, a contrast subchannel 1332, a vacuum subchannel 1335, and an electrical subchannel 1334.

The splitter 1310 is structured to provide fluid communication of saline from the saline channel 1308 to the multiple saline subchannels 1333 in the tube groups 1316, to provide fluid communication of contrast from the contrast channel 1306 to the multiple contrast subchannels 1336 in the tube groups 1316, to provide fluid communication of saline from the single saline channel 1308 to the multiple saline subchannels 1333 in the tube groups 1316, and to provide fluid communication of vacuum from the vacuum channel 1305 to the multiple vacuum subchannels 1335 in the tube groups 1316. Also, the splitter 1310 is structured to provide electrical connection between the electrical channel 1304 and the electrical subchannels 1334 in the tube groups 1316. As illustrated in FIG. 16, the splitter 1310 includes a saline manifold 1333 coupled to the saline channel 1308 and the saline subchannels 1337 of each tube group 1318a-c in the second tubing set 1316, a contrast manifold 1332 coupled to the contrast channel 1308 and the contrast subchannels 1336 of each tube group 1318a-c in the second tubing set 1316, a vacuum manifold 1331 coupled to the vacuum channel 1305 and the vacuum subchannel of each tube group 1318a-c in the second tubing set 1316, and an electrical bus 1330 coupled to the electrical subchannels 1334 of each tube group 1318a-c in the second tubing set 1316. The saline manifold 1333, contrast manifold 1332, and vacuum manifold 1331 can be structured in various configurations that include tubes, splitters, manifolds, or any other structures to provide fluid communication from a single channel in the first tubing set 1302 to multiple subchannels in the second tubing set 1316. The electrical bus 1330 can be any electrical structure that provides electrical communication between the electrical subchannels of the tub groups 1318 in the second tubing set 1316 and the electrical channel 1304 in the first tubing set, including separate electrical wires (leads) or other electrical connections. For example, the electrical subchannels in the tube groups 1318 can include a plurality of electrical leads which are bundled together in the splitter 1310 and form the electrical channel 1304 in the first tubing set. As shown in FIG. 15, the electrical bus 1330 can also electrically connect the electrical channel 1304 of the first tubing set 1302 to the electrical connection 1328 to guide wire mount 1400d. Other embodiments having more or fewer mounts and corresponding connections are also possible. In some embodiments, the splitter 1310 may be referred to as a relay cassette. In some embodiments, the splitter 1310 may be located outside of the sterile field or within the sterile field. In some embodiments, the splitter 1310, second tubing set 1316, and associated hub assemblies (e.g., mounts 1400a, 1400b, and 1440c and associated hubs) can be positioned within the sterile field.

The embodiment of communication channels 1300 illustrated in FIGS. 15 and 16 is designed to minimize the time needed for inspecting the fluid lines. In this example, the fluid communication channels 1300 include a first tubing set 1302 having a proximal end 1312 coupled to the cassette 1200 and a distal end 1314 coupled to a splitter 1310. The fluid communication channels also include a second tubing set 1316 having one or more tube groups 1318, each tube group 1318 having a proximal end 1320 coupled to the splitter 1310 and a distal end 1312 coupled to one of the mounts 1400a-c. However, use of connectors in fluidic systems can cause air bubbles to be formed and/or trapped in a portion of the connector. In some preferred embodiments, advantageously connectors (i.e., releasable connectors) are not used to couple the first tubing set 1302 to the cassette 1200 and the splitter 1310, and are not used to connect the second tubing set 1316 to the splitter 1310. Instead, the first tubing set 1302 is coupled to the cassette 1200 and the splitter 1310 in a persistent coupling (e.g., permanent connection, semi-permanent coupling using glue, clamped fittings, integrally molded portions, and/or other suitable couplings) that it is not meant to be decoupled prior to or during a medical procedure. In preferred embodiments, the first tubing set 1302 and the second tubing set 1316 include transparent tubing. The cassette 1200 can comprise fluid communication channels (including portions of components) configured to communicate saline, contrast, and vacuum in first saline, contrast, and vacuum flow-paths, respectively. For example, as described in reference to FIG. 17A. The splitter 1310 can comprise fluid communication channels configured to communicate saline, contrast, and vacuum in the second saline, contrast, and vacuum flow-paths, respectively. For example, as described in reference to FIG. 16. The fluid communication channels in the cassette 1200 and the splitter 1310 can include one or more tubes, channels (e.g., molded channels), portions of components (e.g., a drip chamber, clot pod, at least a portion of peristaltic saline pump, at least a portion of a contrast pump, robotically controlled valves, etc.) and other suitable fluid communication structures. The proximal end of the first tubing set can be coupled to the cassette 1200 using a persistent coupling. In some embodiments, the proximal end of the first tubing set 1302 extends into the cassette 1200 and forms a portion of a channel in the cassette 1200. The distal end of the first tubing set can be coupled to the splitter 1310 using a persistent coupling. In some embodiments, the distal end of the first tubing set 1302 extend into the splitter 1310 and can form a portion of a channel in the splitter 1310. In some embodiments, a portion of, or all of, the fluid communication channels of the first tubing set 1302 are integrally formed with a portion of, or all of, and the fluid communication channels of the splitter 1310. In an example, tubes forming each of a saline channel 1308, a contrast channel 1306, and a vacuum channel 1305 of the first tubing set 1302 are integrally formed with tubes forming saline, contrast, and vacuum fluid communication channels of the splitter 1310. In some embodiments, a portion of, or all of, the fluid communication channels of the second tubing set 1316 are integrally formed with a portion of, or all of, and the fluid communication channels of the splitter 1310. In an example, a portion, or all of, tubes forming a saline subchannel, a contrast subchannel, and a vacuum subchannel in each of one or more tube groups 1318 are formed to be integral with structures (e.g., tubes) forming the fluid communication channels of the splitter (e.g., channel 1337, channel 1336, channel 1335, manifolds 1333, 1332, 1331). In some embodiments, all or part of the fluid communication channels of the first tubing set 1302, the splitter 1310, and the second tubing set 1316 are formed as in integral structure and are persistently connected. In some embodiments, all or part of the fluid communication channels of the first tubing set 1302, the splitter 1310, the second tubing set 1316 and at least some of the fluid communication structures in the mounts 1400 are formed as an integral structure such that they are persistently connected. In some embodiments, all of the fluid communication channels in the first tubing set 1302, the splitter 1310, the second tubing set 1316 comprise tubing. For example, tubing that is coupled to the cassette 1200, split into multiple tube groups in the splitter, and tubing that is coupled to the splitter and the mounts. In some embodiments, all of the fluid communication channels in the first tubing set 1302, the splitter 1301, the second tubing set 1316 and the mounts 1400 comprise tubing. Some embodiments can include non-persistent coupling of fluid communication channels, for example, it is determined that such connections do not heighten the likelihood trapping air bubbles. In certain embodiments, tubes that are integrally formed with one another may be referred to as “tube sections” that together form a tube.

FIGS. 2, 3, and 4 illustrate some examples of components of a fluidic system including configurations of a cassette. FIGS. 17A-B illustrate examples of a cassette 1200 of the fluidics assembly having a housing 1209 supports portions of a saline subsystem 1201, a contrast subsystem 1202, and a vacuum subsystem 1203 enclosed in, or partially enclosed in, the housing 1209. FIGS. 17A-B also illustrates components of an example of a saline subsystem 1201, a contrast subsystem 1202, and a vacuum subsystem 1203 coupled to the cassette 1200, according to some embodiments. FIG. 17A and FIG. 17B are different embodiments that include many of the same components. The embodiment in FIG. 17A is first described, and then the differences in the embodiment of FIG. 17B are described. Other examples of cassettes may have different components, for example, fewer or more components. The control valves in the saline subsystem 1201, contrast subsystem 1202, and vacuum subsystem 1203 are robotically controlled to be actuated based on a user input, sensor signals received by a controller, and/or executable instructions that one or more hardware processors of a controller are configured to execute to prime the saline, contrast, and vacuum subsystems 1201, 1202, 1203, to prime fluid communication channels coupled to the saline, contrast, and vacuum subsystems 1201, 1202, 1203, prime fluidic channels in hubs/mounts, and provide saline, contrast, and vacuum from the saline, contrast, and vacuum subsystems 1201, 1202, 1203 to the hubs/mounts and ultimately to catheters connected to the hubs/mounts.

One or more saline sources can be coupled to the cassette 1200 to be selectively placed in fluid communication with the saline subsystem 1201. Flow-paths 1206 communicate saline through the saline subsystem 1201. In this example, two saline sources 1106a and 1106b (e.g., saline bags) are coupled to saline input ports 1204a, 1204b for providing a flow of saline to the saline subsystem 1201. The cassette 1200 includes a saline flow-path 1206 that receives saline through input ports 1204 and provides saline to the first tubing set 1302 through saline output port 1230, and also provides saline to the contrast subsystem 1202. Control valve 1214 can be actuated to connect channel 1206c to channel 1206f which is coupled to the vacuum subsystem 1203 vacuum channel 1282a to evacuate saline and air from the saline subsystem 1201 (i.e., channel 1206f can be used as a waste line). Input ports 1204a, 1204b are in fluid communication with a saline first control valve 1220, which can be controlled to select the saline source 1106a, 1106b for providing saline to the cassette 1200. In some embodiments, a connector is used (e.g., a Y-shaped connector), instead of the saline first control valve 1220, to couple the two saline sources 1106a and 1106b to channel 1206a such that the two saline sources 1106a and 1106b are simultaneously in fluid communication with channel 1206a. In some embodiments, a single larger saline source may be used instead of two (or multiple) saline sources. In some embodiments, more than two saline sources can be coupled to channel 1206a, (for example, using two or more Y-shaped connectors, or another connector). In some embodiments the one or more saline sources are each supported by a weight sensor 1210 that is connected to a controller (e.g., in pump station) and provides a signal associated with the weight of each saline source to the controller. The saline subsystem 1201 also includes a peristaltic pump 1213 having an inlet 1207 and an outlet 1208, and an air sensor 1205 positioned between the peristaltic pump 1213 and the first control valve 1220. In some embodiments, the air sensor 1205 is incorporated in the cassette 1200. In other embodiments, the air sensor 1205 is capital equipment (not disposable) and is not included in the cassette 1200, instead it can be included in the pump station and is positioned near channel 1206ab (e.g., near a piece of tubing 1206ab that engages with the air sensor 1205 when the cassette 1200 is coupled to the pump station. In some embodiments, the peristaltic pump is incorporated in the cassette. In some embodiments, a portion of the peristaltic pump 1213 is included in the cassette and a portion of the peristaltic pump is included in the pump station. For example, tubing for the peristaltic pump can be included in the cassette 1200 such that when the cassette 1200 is coupled to the pump station, the tubing interfaces with a movable portion of a peristaltic pump 1213 on the pump station, the movable portion of the peristaltic pump 1213 configured to move saline through the tubing in the cassette 1200 associated with the peristaltic pump to move saline from the saline sources along the saline flow-path 1206 of the saline subsystem 1201. An air sensor 1205 is positioned to sense air in the saline flow-path (channel) 1206ab between the peristaltic pump 1213 and the first control valve 1220, and is configured to generate a signal indicative of air detected. In some embodiments, the air sensor 1205 is capital equipment and located (for example, on the pump station). In such embodiments, channel 1206ab can include a portion of tubing that engages into the air sensor. In some embodiments, the air sensor is located in the cassette 1200 and is connected to the electrical interface 1236 such that a signal generated by the air sensor 1205 can be provided to the pump station (or another controller).

The saline subsystem 1201 also includes a pressure sensor 1212, positioned downstream of the peristaltic pump 1213, and configured to generate a signal indicative of pressure in the saline flow-path. The saline subsystem further includes a saline second control valve 1214 that is configured to selectively, as controlled by a controller, provide saline to a channel 1222 which is connected to the vacuum subsystem 1203. In operation, a controller may align the second control valve 1214 to diverge the saline flow-path to the vacuum subsystem 1203 to prime the upstream portion of the saline flow-path 1206a-c, for example, when the saline sources are first connected to the saline flow-path or when the saline source is switched from the first saline source 1106a a second saline source 1106b. The saline subsystem 1201 also includes a saline third control valve 1218 that is configured to selectively, as controlled by a controller, provide saline to a channel 1256 which is connected to the contrast subsystem 1202 for priming the contrast subsystem 1202 with saline. In a normal operational configuration for providing saline to catheters, a controller aligns the first control valve 1220 to receive saline from one of the two connected saline sources 1106, aligns the second control valve 1214 (“saline prime stopcock”) to provide saline from peristaltic pump 1213 to the third control valve 1218, and the lines the third control valve 1218 to provide saline to the saline output port 1230 to flow into the first tubing set 1302.

Still referring to the example illustrated in FIG. 17A, the contrast subsystem 1202 includes a contrast flow-path 1244 that receives contrast from a contrast source 1108 through contrast inlet port 1242 along flow-path 1244ab, communicates contrast to the first tubing set 1302 via contrast outlet port 1264 along flow-path 1244bd, and communicates air and/or contrast from a contrast pump 810 to the vacuum subsystem 1203 along flow-path 1244c. In some embodiments, the contrast subsystem 1202 includes a control valve 1211 (“contrast refill stopcock”) coupled to channels 1244abd that can be actuated to connect the contrast pump 810 to the contrast source 1108 (connecting channels 1244b and 1244a), or connecting the contrast pump 810 to channel 1244d and the contrast channel of the first tubing set 1302. An air sensor 1246 is positioned along the contrast flow-path between the contrast source 1108 and the contrast pump 810, and is configured to sense air in the contrast flow-path (channel) 1244a and generate a signal indicative of air being in the contrast flow-path. In some embodiments, the air sensor 1246 is capital equipment and located (for example, on the pump station). In such embodiments, channel 1244a can include a portion of tubing that engages into the air sensor 1246. In some embodiments, the air sensor 1246 is located in the cassette 1200 and is connected to the electrical interface 1236 such that a signal generated by the air sensor 1246 can be provided to the pump station (or another controller). In some embodiments, based on receiving a signal from the air sensor 1246 indicating presence of air in flow-path 1244a, the controller may pause contrast pump operation, indicate another contrast source is needed on a user display or with an audible alarm. When a new contrast source is attached to contrast inlet port 1242, in some embodiments the controller can initiate a priming sequence that includes actuating the contrast control valve 1247 to provide vacuum to the contrast pump 810 (connecting channel 1244c to port 1250) and actuating valve 1211 to connect channels 1244a and 1244b to expel air in the contrast pump 810 and channels 1244ab through channel 1244c. When changing contrast sources (bottles), channel 1244b and the contrast pump (syringe) 810 are already full of contrast, but 1244a has air in it. Instead of pulling air through the primed syringe, control valve 1247 can be actuated to block channel 1244c from being in fluid communication with port 1250 (if not already so aligned) and control valve 1211 can be actuated to connect channel 1244a to be in fluid communication with contrast pump outlet port 1254, and the contrast pump 810 can be actuated to push air in channel 1244a into the contrast source 1108.

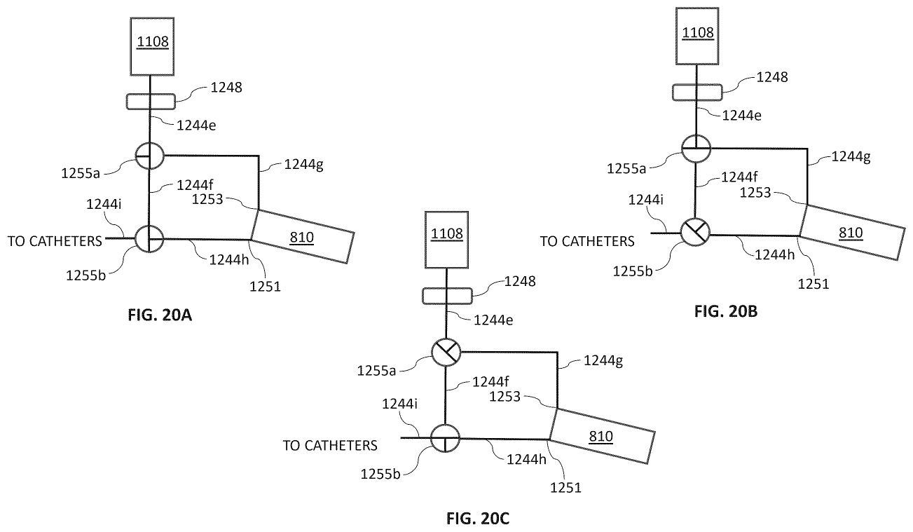

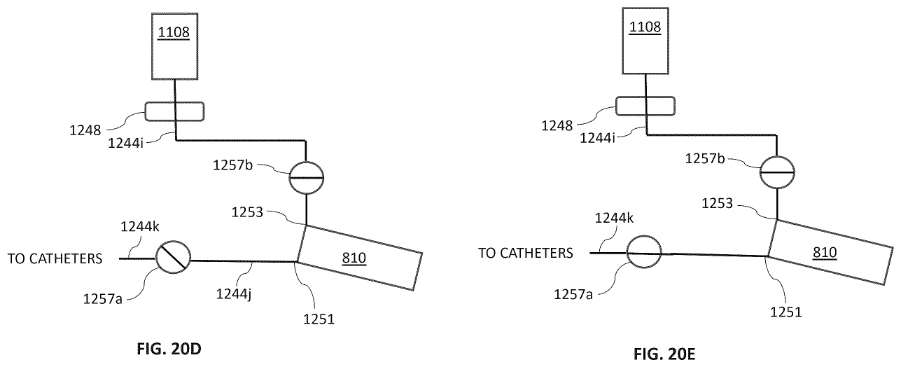

The contrast pump 810 receives contrast into a chamber in the contrast pump 810 from the contrast source 1108 through contrast (outlet) port 1254 to fill the contrast pump 810. A contrast pump port 1250 connects the contrast pump 810 to channel 1244c, 1249 which is connected to the vacuum subsystem 1203, and may be used to remove air from the contrast pump 810. Contrast control valve 1247 is positioned between contrast pump port 1250 and the vacuum subsystem, and is selectively controlled by a controller to align the contrast control valve 1247 to open or to shut the air/contrast flow-path between the contrast pump 810 and the vacuum subsystem 1203. When filling the contrast pump 810 with contrast from contrast source 1108, in some embodiments, the contrast control valve 1247 can be opened to provide vacuum to the contrast pump 810 through port 1250 to evacuate air in the contrast pump 810 along flow-path 1244c, and help cause contrast from the contrast source to flow along flow-paths 1244a and 1244b into port 1254 to fill the pump 810. In an example, the contrast control valve 1247 is opened for a predetermined amount of time to prime the contrast pump 810. In other examples, vacuum is not used during the priming process. For example, once the contrast pump 810 is at least partially filled with contrast, the contrast pump can be actuated to expel any air in the pump through channel 1244c and/or through channel 1244b (e.g., to the contrast source). The contrast control valve 1247 is closed for operation of the contrast pump 810. FIG. 17A illustrates one example of a contrast subsystem 1202, but other configurations are also contemplated. For example, FIGS. 20A-C and FIGS. 20D-E illustrate two other configurations that also can be incorporated in the cassette 1200.

The vacuum subsystem 1203 of the cassette 1200 includes a vacuum port 1271 coupled to a vacuum canister 1270, which can be coupled to a vacuum filter 1269, and a vacuum source assembly (collectively “vacuum source”) 1267 which can include a vacuum pump, a vacuum regulator, and other components for providing a consistent vacuum controlled robotically. In some embodiments, the vacuum filter 1269 (positioned between the vacuum cannister 1270 and the vacuum source 1267) is incorporated in the vacuum cannister 1270, for example, on (or in) the lid of the vacuum cannister 1270, which is advantageous as it can be easily disposed of with the vacuum cannister 1270 instead of having another element outside of the vacuum cannister to properly dispose of. The vacuum cannister (sometimes referred to as a “waste cannister”) is a receptable that includes a cavity for collecting material aspirated from a fluidic line, a patient, or a catheter. The vacuum source 1267 can be controlled by a controller to provide a desired amount of vacuum to a vacuum flow-path. In some embodiments, a clamp 1307 can be attached to the vacuum line to close off the vacuum line, for example, in case there is a need to stop vacuum and the vacuum pump and control valves controlling aspiration cannot be actuated, or cannot be quickly actuated. The clamp can be manually operated, or operated by an actuator controlled by the system. In various embodiments, the clamp 1307 can be placed at any location that is accessible (e.g., outside of the cassette) and between the vacuum pump 1267 and the splitter 1301, for example, between the vacuum cannister 1270 and the cassette 1200.

As illustrated in FIG. 17A, in this example the vacuum canister 1270, and vacuum source 1267 are positioned outside of the cassette 1200. Vacuum flow-path 1282 extends from the vacuum inlet port 1271, through drip pod 1272 to vacuum port 1289, which is coupled to the first tubing set 1302. A portion of the vacuum flow-path 1282 extends from the vacuum port 1271 to the drip pod 1272. The contrast subsystem 1202 and the saline subsystem 1201 are connected to the vacuum flow-path 1282 through channels 1249 and 1222, respectively, connecting between the drip pod 1272 and vacuum port 1271 such that fluid and air from the saline subsystem 1201 and the contrast subsystem 1202 do not go through the drip pod 1272, instead flowing through vacuum port 1271 and into vacuum canister 1270. The drip pod 1272 includes clot pod 1273, drip chamber 1278, and a first three-way control valve 1275 positioned in the vacuum flow-path 1282 between the drip chamber 1278 and the clot pod 1273. In various embodiments, the clot pod 1273 can have different shapes (e.g., rectangular, circular, etc.). The first control valve 1275 is controlled by a controller to align the vacuum flow-path 1282 to connect clot pod inlet line (or “channel”) 1274 to either the drip chamber outlet line 1276 or the drip chamber bypass line 1277. The first control valve 1275 can also be controlled by a controller to align the vacuum flow-path 1282 to neither the drip chamber outlet line 1276 or the drip chamber bypass line 1277. Drip chamber inlet line 1279 connects the vacuum flow-path to second three-way control valve 1280, which is controlled by a controller to align the vacuum flow-path 1282 to vacuum port 1289 and the first tubing set 1302, or to the local atmosphere. A pressure sensor 1283 is positioned along the flow-path 1282 between control valve 1280 and vacuum port 1289. Pressure sensor 1283 also connected to the electrical interface 1236, and is configured to detect pressure in the vacuum flow-path 1282 and provide a signal indicative of the detected pressure to the electrical interface 1236.

At least a portion of the drip pod 1272 can be transparent to allow a medical practitioner to view the drip chamber 1278 and the clot pod 1273. For example, to see a fluid flow into the drip chamber and to determine a level of fluid accumulated in the drip chamber, and to view and assess material captured in the clot pod. In an example, the drip pod 1272 can be structured to be positioned along a surface of the cassette 1200. In some embodiments, one side of the drip pod 1272 can be transparent to such that the drip chamber and the clot pod are visible to a medical practitioner when the cassette is mounted on the pump station, and the opposite side is transparent or translucent to allow light illuminate the drip chamber and/or clot pod for easier viewing. In other embodiments, at least one wall of the drip pod 1272, proximal to a surface of the cassette 1200 that is coupled to a the pump station (i.e., a proximal surface of the cassette) is transparent so that the drip chamber 1278 and/or the clot pod 1273 can be imaged, through proximal surface of the cassette and through the transparent surface of the drip pod 1272, by an imaging device in the pump station. For example, an imaging sensor (e.g., a camera) 1292 can be positioned to generate an image of the clot pod 1273 and/or the drip chamber 1278, and the image can be displayed locally or remotely to provide information on the contents of the clot pod 1273 and/or the drip chamber 1273. This is advantageous and critical for aspiration and back-bleeding procedures when the medical practitioner needs to know the contents of the clot pod (e.g., during clot aspiration) and needs to know the amount and/or consistency of fluid entering the drip chamber 1273 (e.g., during back-bleeding). In this embodiment, the imaging sensor 1292 is not included in the cassette 1200, but instead the imaging sensor 1292 can be incorporated in the pump station or another suitable location (locally) such that it can generate an image of the clot pod 1273 and/or drip chamber 1278. In some embodiments, the imaging sensor 1292 can be incorporated in the cassette, however, having the imaging sensor positioned in the disposable cassette may increase cost. In embodiments where the imaging sensor is disposed external to the cassette 1200, a portion of the cassette 1200 can be transparent to allow the imaging sensor 1292 to image the drip pod 1272, or a portion thereof.

Saline subsystem 1201 can include air sensor 1205 and pressure sensor 1212, the contrast subsystem 1202 includes air sensor 1246, and the vacuum subsystem 1203 includes pressure sensor 1283. Signals from these sensors are received by a controller, which can use these signals to align control valves in the cassette 1200 to vary the saline, contrast, and vacuum flow-paths in the cassette and perform processes described herein, and other processes. A purpose of these sensors is to detect potentially hazardous situations, for example, running out of a fluid source, a kink or occlusion in a fluid line, etc. Although the saline and contrast air sensors 1205, 1246 can be located in a pump station and engage a portion of saline or contrast tubing in the cassette, or they can be located in the cassette, such air sensors can be considered and referred to as being a part of the saline subsystem 1201 or the contrast subsystem 1202.

The configuration of the cassette 1200 in FIG. 17A has certain different features than cassettes described above, which can provide operational and cost advantages. For example, although in some embodiments the vacuum canister may be positioned in the cassette, in FIG. 17A the vacuum canister 1270 (and vacuum regulator 1269 and the vacuum pump 1267) are positioned outside of the cassette in the line from the vacuum canister 1270 is coupled to the cassette at port 1271. This may allow the disposable cassette to be less expensive, and allow various configurations of vacuum source assemblies (e.g., the vacuum canister, the vacuum regulator, and the vacuum pump) to be used with the system, and any changes or improvements to the vacuum canister will not affect the design of the cassette. The configuration of the cassette 1200 in FIG. 17A includes two saline sources 1106a, 1106b in a control valve 1220 that a controller can align to select either one of the saline sources, either based on a manual input or automatically based on the sensed condition (e.g., the signal from weight sensor 1210 that a saline source is depleted below a predetermined threshold, a signal from air sensor 1205 that air is present in the saline flow-path 1206). The saline subsystem 1201 in FIG. 17A includes channel 1222 from the saline subsystem to the vacuum subsystem 1203 which allows for a simplified priming procedure. In some embodiments, if air is detected, the controller can be configured to automatically, or based on user input, to prime the saline subsystem by directing the saline flow-path 1206 upstream of the second control valve 1214 to vacuum such that saline and the air in the saline subsystem 1201 upstream of the second control valve 1214 is evacuated into the vacuum canister 1270. In this embodiment, control valves that the controller can selectively align to provide contrast from the contrast subsystem 1202 to one or more catheters are located in the mounts as shown in an example, in FIG. 19 (e.g., control valve 1426). Similarly, control valves that the controller can align to provide saline from the saline subsystem 1201 a catheter located in the mounts 1400 (e.g., FIG. 19). Having these control valves in the mounts also advantageously obviates the need for having multiple individually controllable saline lines and contrast lines extending from the cassette 1200 to the mounts 1400, such that the fluid communication channels 1300 (FIG. 16) are also simplified and include less tubing. Also, unlike some of the previous embodiments described above, the example configuration illustrated in FIG. 17A advantageously does not include saline lines or control valves for providing saline to a femoral sheath, thus further simplifying the design of the cassette 1200 and may lower manufacturing costs. Instead, in this example, saline can be provided to a femoral sheath from a separate system (not shown here).

FIG. 17B is a schematic illustrating another example of a cassette of the fluidics assembly, the cassette including a housing that can support, all or part of a saline subsystem 1201, a contrast subsystem 1202, and a vacuum subsystem 1203. In the embodiment shown in FIG. 17B, although most of the components can be the same or similar to the components illustrated in FIG. 17A, there are a few differences. In the embodiment of FIG. 17B, contrast control valve (contrast prime stopcock) 1247 can be a three-way valve instead of a two-way valve due to ease of making and/or implementation. Also, control valve 1214 (saline prime stopcock) is positioned between control valve 1218 and control valve (contrast refill stopcock) 1211, such that it can connect channel 1256 with channel 1206f. In this configuration, air and saline can be aspirated from the saline flowpath through channels 1206b, 1206c, 1206d, and 1206e to the vacuum canister 1270 via control valve 1214. Also, air sensor 1205 is located downstream of the peristaltic pump 1213 (instead of being between the peristaltic pump and control valve 1220). At this location, the air sensor 1205 can sense air that is in the saline flowpath downstream of the peristaltic pump 1213 to provide a better determination that the saline flowpath contains no air. The air sensor 1205 and the air sensor 1246 can be included in the cassette 1200 and be disposable, in some embodiments. In other embodiments, an air sensor 1205 and air sensor 1246 are part of the pump station interface with the cassette 1200 and are part of capital equipment (i.e., not disposable). In the embodiment illustrated in FIG. 17B, the control valve 1220 has been replaced with a Y-connector (or Wye-connector) 1220x such that both of the saline sources 1106a, 1106b provide saline simultaneously. In some embodiments, the fluidics system does not employ a weight sensor 1210.

In reference to FIGS. 14-17B, examples of a fluidics management system can comprise a cassette configured to receive saline from a saline source (e.g., two or more saline sources), receive contrast from a contrast source, and receive vacuum from a vacuum source. The cassette can include saline subsystem having a first saline flow-path; a contrast subsystem having a first contrast flow-path; and a vacuum subsystem including a first vacuum flow-path. The first saline, contrast, and vacuum flow-paths can include channels, tubes, line, and portions of other components (e.g., one or more of a valve, drip chamber, clot pod, pump, etc. in the cassette). The fluidics management system can also include a splitter having a second saline flow-path, a second contrast flow-path, and a second vacuum flow-path. Each of the second saline, contrast, and vacuum flow-paths has single proximal end and a plurality of distal ends. The single proximal end of the second saline flow-path is in fluid communication with a single saline channel in a first tubing set, the proximal end of the second contrast flow-path is in fluid communication with a single channel of the first tubing set. The second vacuum flow-path proximal end is in fluid communication with the single vacuum channel of the first tubing set. The first tubing set has a first length, as measured between the cassette and the splitter, and is coupled to the cassette and splitter. The first tubing set includes a single saline channel coupled to the first saline flow-path and the proximal end of the second saline flow-path, the single contrast channel coupled to the first contrast flow-path and the proximal end of the second contrast flow-path, and a single vacuum channel coupled to the first vacuum flow-path and the proximal end of the second vacuum flow-path. The fluidics management system can also include two or more mounts, for example, three mounts, or four mounts. The mounts can be configured in various ways to provide saline, contrast, and vacuum to catheters coupled to each respective mount. Each mount can be configured to have a third saline flow-path, a third contrast flow-path, and a third vacuum flow-path to provide saline, contrast and vacuum to a catheter via a connector configured to couple to a catheter. In some embodiments, the catheter is coupled to another assembly (e.g., a hub) which is coupled to the mount, and the lumen of the catheter is coupled to the connector to be in fluid communication with the connector. A mount can include a plurality of robotically actuated control valves controlled by a control system to selectively align the third saline flow-path, the third contrast flow-path, and the third vacuum flow-path to be in fluid communication with the connector. The fluidics management system can also include a second tubing set having a second length, the second tubing set comprising a plurality of tube groups, each tube group coupled to the splitter on a proximal end of the tube group and to one of the two or more mounts on a distal end of the tube group. Each tube group including a saline subchannel coupled to the distal end of the second saline flow-path of the splitter, a contrast subchannel coupled to the distal end of the second contrast flow-path of the splitter, and a vacuum subchannel coupled to the distal end of the second vacuum flow-path of the splitter. The first tubing set has a first length and the second tubing set has a second length, the first length being greater than the second length to minimize the amount of tubing that needs to be visually inspected for air bubbles. In some examples, first tubing set has a first length and the second tubing set has a second length, and the first length of the first tubing set is at least two times as long as the second length of the second tubing set.

Priming of the saline subsystem 1201 can include robotically actuating control valves of the saline subsystem 1201, robotically actuating a peristaltic saline pump, and robotically controlling vacuum in the vacuum subsystem to perform a priming process. In one example of priming of the saline subsystem 1201, the cassette 1200 is coupled to a pump station and the saline sources 1106 are coupled to the saline subsystem. To be able to switch from one saline source to another connected saline source seamlessly, channels connecting both saline sources to the saline subsystem 1201 must be primed. Control valve 1220 can be robotically controlled to couple a first saline source to peristaltic pump inlet 1207. The control valve 1214 can be aligned to connect the peristaltic pump outlet 1208 to channel 1222 which is connected to the vacuum subsystem 1203 to purge air from the saline subsystem. The vacuum subsystem can be robotically controlled to provide vacuum to channel 1222 to purge air during priming, or to not provide vacuum and instead channel 1222 and cannister 1270 is used to purge air without applying vacuum using channel 1222. In this configuration, the saline peristaltic pump can be run (e.g., for a predetermined time) to evacuate air in the saline flow-path from the first saline source to control valve 1214. During this priming, signals from air sensor 1205 should eventually indicate no air is detected. The control valve can be actuated to connect a second saline source to peristaltic pump inlet 1207. The control valve 1214 can continue to be aligned to connect the peristaltic pump outlet 1208 to channel 1222 which is connected to the vacuum subsystem 1203. The vacuum subsystem can again be robotically controlled to provide vacuum to channel 1222. In this configuration, the saline peristaltic pump can be run (e.g., for a predetermined time) to evacuate air in the saline flow-path from the second saline source to control valve 1214. During this priming, signals from air sensor 1205 should eventually indicate no air is detected. Control valve 1214 can be actuated to connect the outlet port 1208 to the second control valve 1218 (“saline bypass stopcock”), which can be actuated to connect the outlet port 1208 to the contrast subsystem 1202 for priming the contrast subsystem, or connect outlet port 1208 to a saline communication channel to provide saline to a plurality of mounts 1400.

Priming of the contrast subsystem 1202 can include robotically actuating control valve 1247, robotically actuating contrast pump 810, and robotically controlling the vacuum subsystem 1203 to perform a priming process. In an example of a priming process of the contrast subsystem 1202, the contrast subsystem 1202 is connected to a contrast source 1108. Control valve 1247 is robotically actuated to connect outlet port 1250 to the vacuum subsystem 1203. In some embodiments of a contrast priming process, the vacuum pump 1267 is robotically actuated to provide vacuum to the contrast pump 810 and the contrast flow-path 1244, which evacuates air from the contrast flow-path (channel) 1244 and the contrast pump 810 as it fills with contrast. In other embodiments of a contrast priming process, vacuum is not provided to the contrast pump 810 via channel 1244, instead channel 1244 is used to evacuate air which evacuate air from channels 1244 and the contrast pump 810 as the pump is filled with contrast. Subsequently, control valve 1247 is closed and the contrast pump 810 can be robotically actuated to provide contrast to a prime a contrast communication channel, to prime the plurality of mounts, and to provide contrast to the plurality of mounts 1400. As part of priming the contrast communication channels connect the cassette to the mounts, control valve 1218 can robotically actuated to connect outlet port 1208 to the contrast flow-path 1244, and the saline peristaltic pump can be actuated to provide saline to the contrast subsystem such that it fills the contrast communication channels to the mounts and in the mounts. The vacuum subsystem 1203 can be robotically controlled to provide vacuum to the mounts 1400 via a vacuum communication channel. In each mount, one or more control valves can be robotically actuated to place a contrast channel in the mount, that is being provided saline from the cassette, in fluid communication with a vacuum channel to facilitate priming the contrast communication channel between the cassette and the mount, and in the mount, with saline to evacuate all air in these channels. Subsequently, control valve 1218 can be actuated to connect the outlet port 1208 with saline outlet port 1230 and the saline communication channel that is coupled to the mounts.

As indicated above for other embodiments of a fluidics system, saline and contrast channels in the fluidics assembly 1100 and in hub assemblies (e.g., mounts and/or hubs) are also primed prior to use in a medical procedure. In an example, once the saline subsystem 1201 is primed, the control valve 1218 be actuated to connect the outlet of the saline pump to the saline channels in the fluidics assembly 1100 and the saline pump 1213 can be actuated to provide saline to fill the saline 1308, the saline manifold 1333, the saline subchannels 1337 of each tube group in the second tubing set 1316, and the saline channel 1412 (e.g., saline channels 1412a-c) of each mount 1400 with saline. To facilitate the flow of saline through these saline channels, the control valves 1426 (e.g., 1426a-c) and 1428 (e.g., control valves 1428a-c) can be actuated to connect the saline channel 1412 with the vacuum channel 1416 (e.g., vacuum channels 1416a-c) while controlling the vacuum subsystem 1203 to provide vacuum to the mounts 1400. Once the saline subsystem 1201 is primed, the control valve 1218 be actuated to connect the outlet of the saline pump 1213 to the contrast subsystem 1202, and the saline pump 1213 can be actuated to provide saline into the contrast subsystem 1202, to fill the contrast channel 1306, the contrast manifold 1333, the contrast subchannels 1336 of the second tubing set, and the contrast channel 1414 (e.g., contrast channels 1414a-c) of each mount 1400 with saline. To facilitate the flow of saline through the contrast channels, the control valves 1426 and 1428 can be actuated to connect the contrast channel 1414 with the vacuum channel 1416 while controlling the vacuum subsystem to provide vacuum to the mount. A controller can use signals received from the saline and contrast air sensors 1418 (e.g., sensors 1418a-c), 1420 (e.g., sensors 1420a-c) to determine when no air is detected in the saline channel 1412 and the contrast channel 1414, indicating the saline and contrast channels between the mounts and the cassette are primed with saline. Similar to the priming processes described above, once the contrast channels from the cassette to the mounts are primed with saline, and the contrast channel 1414 is primed with saline, Channels of the fluidic assembly can also be prime.

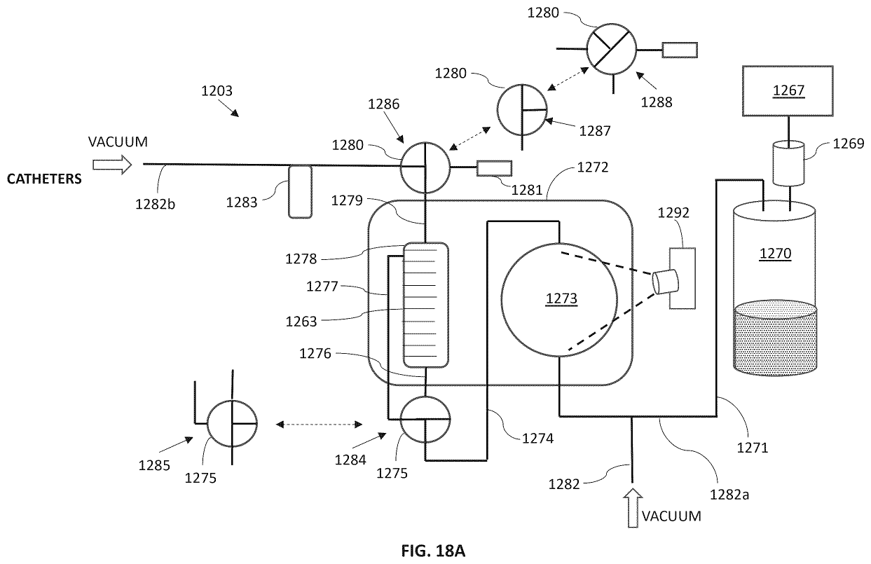

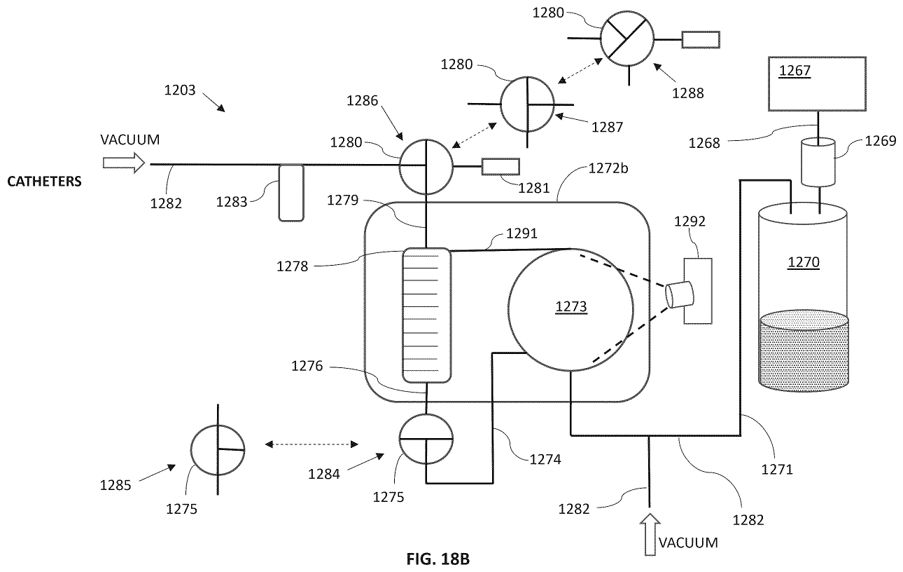

FIG. 18A is a schematic illustrating an example of drip pod 1272 shown in FIGS. 17A-B, and illustrates positions of the control valves 1275, 1280 to connect the vacuum flow-path 1282 through different channels of the drip pod 1272 and to the first tubing set 1302. When control valve 1275 is aligned in position 1284, the vacuum flow-path is though the drip chamber bypass channel 1277. This allows vacuum to be in the drip chamber 1278 but does not flush fluid straight through the drip chamber 1278. When the first control valve 1280 is aligned in position 1284, the controller can control the vacuum regulator to provide the vacuum to be at a lower vacuum level (less vacuum) compared to when the first control valve 1275 is aligned in position 1285. In this configuration, when the second control valve 1280 is aligned in position 1286 and the vacuum flow-path in the associated mount is aligned to provide vacuum to the attached catheter, the low vacuum causes a slow flow of fluid from the catheter to the vacuum subsystem 1203 such that the fluid drips into the drip chamber 1278, and the cumulative amount of fluid flowing into the drip chamber 1278 can be visually determined using drip chamber graduation lines 1263, along the length of the drip chamber 1278, as the fluid fills the drip chamber 1278. When the first control valve 1275 is aligned in position 1285, the vacuum flow-path is through the drip chamber 1278, through drip chamber outlet line 1276, through the first control valve 1275 and to the clot pod 1273. When the first control valve 1275 is aligned in position 1285 and the second control valve is aligned in position 1286, and the associated mount is aligned to provide vacuum to the catheter, a controller can control the amount of vacuum to be high vacuum (a higher amount than when in position 1284) such that fluid flows more rapidly through the drip chamber 1278, through the first control valve 1275 to the clot pod 1273, and further to the vacuum canister 1270. Such a configuration can be used, for example, for aspirating a clot or providing a saline flush of the clot pod 1273. A controller can align the second control valve 1280 to align vacuum flow-path to a first position 1286 to provide vacuum to the associated mount 1400, to position 1287 to provide vacuum flow-path to atmosphere (for example, to quickly flush out the drip chamber 1278 and the clot pod 1273), or to position 1288 to cut off the vacuum flow-path from an associated mount and atmosphere 128 (for example, when receiving fluid from the saline subsystem 1201 or the contrast subsystem 1202.

FIG. 18B is a schematic illustrating another example of a drip pod. This example is similar to the example illustrated in FIG. 18A except instead of having a drip chamber bypass line 1277, there is a line 1291 from the clot pod 1273 to the distal end of the drip chamber 1278 such that the vacuum flow-path can go directly from the clot pod 1273 to the drip chamber 1278, and the first control valve 1275 can be aligned by a controller to be in a first position 1284 to block vacuum flow-path to the drip chamber 1278 and to be in a second position 1285 to connect vacuum flow-path to the drip chamber 1278. Accordingly, in various embodiments the cassette further includes a vacuum subsystem having a clot pod. The clot pod can have at least one transparent surface positioned in/on the cassette to be visible from outside of the cassette. The cassette can further comprise a vacuum subsystem with a drip chamber in fluid communication with the first vacuum flow-path. The drip chamber can have at least one transparent surface. Also, the drip chamber can be positioned in/on the cassette to be visible from outside of the cassette. The drip chamber can be positioned in the first vacuum flow-path between the clot pod and the first tubing set. The vacuum subsystem can include a plurality of robotically actuated valves configured to be controlled by a control system for controlling the vacuum flow-path through the drip chamber and the clot pod. As an example, the plurality of robotically actuated valves includes a first valve positioned in the first vacuum flow-path between the drip chamber and the clot pod and a second valve positioned on an opposite side of the drip chamber in the first vacuum flow-path between the drip chamber and the first tubing set, wherein first valve and second valve can be aligned to control the flow of fluid and material from the mounts through the drip chamber and the clot pod. In various embodiments, the control system includes a controller, for example, controller 2230, controller 2220, or the pump station (e.g., a controller in the pump station). The control system can include a controller including non-transitory computer storage medium configured to store computer-executable instructions, and including one or more computer hardware processors in communication with the non-transitory computer storage medium, the one or more computer hardware processors configured to execute the computer-executable instructions to control the pump station to actuate the saline subsystem, the contrast subsystem, and the vacuum subsystem, to independently provide to each hub in the set of hubs contrast, saline, and vacuum via the fluid communication system. Such a controller can be configured to robotically actuate valves and pumps in the fluidics management system at least partially based on a user input, sensed characteristics, and/or executable instructions.

FIG. 19 is a schematic illustrating an example of a fluidic system components in one or more mounts 1400 coupled to the second tubing set 1316. This example includes three mounts 1400a-c that have the same components relating to the fluidic system. Each mount 1400 has fluidic connections to the second tubing set 1316 to receive saline, contrast, and vacuum, and an electrical connection that includes one or more electrical leads, which are coupled to one or more sensors in the mounts 1400 to communicate signals from one or more sensors in the mount to the cassette, and ultimately to a controller (e.g., controller 2220) when the cassette is coupled to the pump station. The controller uses the received signals to align valves in the mounts to provide saline, contrast, and aspiration automatically or semi-automatically (e.g., based on a user input) as needed during different portions of a medical procedure. In this example, mount 1400a has a common connector 1434a for providing saline, contrast, and vacuum to the lumen of catheter 1402a via hub 724a, mount 1400b has a connector 1434b for providing saline, contrast, and vacuum to the lumen of catheter 1402b via hub 724b, and mount 1400c has a connector 1434c for providing saline, contrast, and vacuum to the lumen of catheter 1402c via hub 724c. The connectors 1434 can be in fluid communication with the lumens of catheters 1402 by coupling the connectors 1434 to a corresponding connector on hubs 724 that the catheters 1402 are coupled to, where the hubs 724a-c can include a fluid communication channel from the connector 1434 to the lumen of the catheter 1402 coupled to the hub 724. Such hubs 724 can be removably coupled to the mounts 1400. The hub can include structure that operates as a hemostasis valve 928, such that the hub 724 can provide fluid to the lumen of a first catheter coupled to the hub that for discharging at the distal end of the first catheter, or can provide vacuum to the lumen of a first catheter coupled to the hub for providing aspiration at the distal end of the first catheter, while another elongated device is positioned partially or fully in the lumen of the first catheter in a concentric nested configuration. For example, when catheter 1402b is positioned in the lumen of catheter 1402a, when catheter 1402c is positioned in the lumen of catheter 1402b, or when an elongated device (e.g., a catheter, a guidewire, etc.) is positioned in the lumen of catheter 1402c. In some embodiments, catheter 1402a is a guide catheter, catheter 1402 is a procedure catheter, and catheter 1402c is an access (or insert) catheter.

Still referring to the embodiment illustrated in FIG. 19, each mount 1400 (e.g., mounts 1400a-c) can include a saline port 1406 (e.g., saline ports 1406a-c), a contrast port 1408 (e.g., contrast ports 1408a-c), a vacuum port 1410 (e.g., vacuum ports 1410a-c), and an electrical connection 1411 (e.g., electrical connections 1411a-c) that are coupled to saline subchannel 1337, contrast subchannel 1336, vacuum subchannel 1335, and electrical channel 1334, respectively, of a tube group 1318 (of second tubing set 1316) coupled to the mount 1400. Preferably, the mount 1400 is coupled to the tube group 1318 without using connectors, for example, in a persistent connection that is not meant to be decoupled before or during its use in a medical procedure, which may help eliminate bubbles from the fluidic channel during priming. In other embodiments, a connector can be used. An air sensor 1418 is positioned along the saline channel (flow-path) 1412 and is in electrical communication with electrical connection 1411. The air sensor 1418 is configured to detect air in the saline channel 1412 and generate a signal indicative of detecting air which is provided to the cassette 1200 and ultimately a controller, via the second tubing set 1316, splitter 1310, first tubing set 1302, and the cassette 1200. An air sensor 1420 is positioned along contrast channel (flow-path) 1414 and is in electrical communication with electrical connection 1411. The air sensor 1420 is configured to detect air in the contrast channel 1414 and generate a signal indicative of detecting air which is provided to cassette 1200 and ultimately a controller via the second tubing set 1316, splitter 1310, first tubing set 1302, and the cassette 1200.

Mount 1400 includes a first control valve 1426 that is controllable by a controller to align saline channel 1412, or contrast channel 1414, to a second control valve 1428. Check valve 1422 (e.g., check valves 1422a-c) in the saline channel 1412 prevents any upstream fluid flow on the saline channel 1412. Along the saline channel 1412 between the first control valve 1426 and the check valve 1422 is a saline drip channel (saline restricted-flow channel) 1424 which connects the saline channel 1412 upstream of the first control valve 1426 a fluid flow-path to the second control valve 1428, bypassing the first control valve 1426. In this configuration, at least some saline that enters the mount 1400 can flow to via the saline restricted-flow channel 1424 to the second control valve 1428 regardless of the position of the first control valve 1426. The saline drip flow-path includes a saline restricted-flow channel 1424 designed to allow a desired flow-rate of saline (e. g, mL/minute) to flow to the connector 1434 and to the lumen of a catheter in fluid communication with the connector 1434 when the second control valve 1428 is aligned to provide saline or contrast to the connector 1434, regardless of the alignment of the first control valve 1426. The saline restricted-flow channel 1424 is configured to provide a lower flow-rate of saline than the saline channel 1412 provides to the saline-contrast channel 1421 through the first control valve 1426. The saline restricted-flow channel 1424 can have, for example, a smaller cross-sectional dimension then the cross-sectional dimension of the flow-path from the saline channel 1412 to the saline-contrast channel 1421 through the first control valve 1426. As an example, the saline restricted-flow channel 1424 can have narrow portion of the saline drip flow-path designed such that the saline restricted-flow channel 1424 provides about 1 mL/minute of saline to the lumen of an associated catheter. For example, in the range of about 0.85 mL/minute to about 1.35 mL/minute. In systems with multiple catheters, the catheters can be positioned in a concentric configuration, that is, such that catheter 1402c can be positioned at least partially in the lumen of catheter 1402b, and catheter 1402b can be positioned at least partially in the lumen of catheter 1402a. In the example illustrated in FIG. 19, flow restrictor FR1 is associated with catheter 1402a, flow restrictor FR2 is associated with catheter 1402b, and flow restrictor FR3 is associated with catheter 1402c. As an example, the saline restricted-flow channel 1424a can have a flow restrictor (value, size) FR1 designed such that the saline restricted-flow channel 1424 provides about 1 mL/minute of saline to the lumen of a catheter having a lumen inner diameter (ID) of about 0.088 inches (for example, a Zoom 88 catheter). As an example, the saline restricted-flow channel 1424b can have a flow restrictor FR2 designed such that the saline restricted-flow channel 1424b provides about 1 mL/minute of saline to the lumen of a catheter having an ID of about 0.071 inches (for example, a Zoom 71 catheter). As an example, the saline restricted-flow channel 1424c can have a flow restrictor FR3 designed such that the saline restricted-flow channel 1424c provides about 1 ml of saline to the lumen of a catheter having a lumen inner diameter (ID) of about 0.035 inches (for example, a Zoom 35 catheter). The flow restrictor FR1, FR2, FR3 can be all about the same size (e.g., having the same or about the same ID), or can be sized differently based on the catheter size a flow restrictor is associated with such that a saline drip flow of about 1mL/minute is achieved to the catheter in fluid communication with the connector 1434.

The second control valve 1428 is a three-way valve that connects either vacuum to or saline/contrast to connector 1434 via a fluid primary channel 1432 (e.g., primary fluid channels 1432a-c). The primary channel provides saline, contrast and vacuum to a catheter coupled to the mount 1400, for example, provides saline, contrast and vacuum to the lumen of the catheter through connector 1434. The primary channel also receives material (e.g., fluids, clots, etc.) from a catheter coupled to the mount 1400 when vacuum is provided to the catheter. Pressure sensor 929 (e.g., a hemodynamic pressure sensor) is positioned to detect pressure in the primary channel 1432 and is in electrical communication with electrical connection 1411. Pressure sensor 929 is configured to generate a signal, indicative of the detected pressure, which is provided to cassette 1200 and ultimately a controller via the second tubing set 1316, splitter 1310, first tubing set 1302, and the cassette 1200. The configuration of mounts 1400 includes several advantages over previous configurations described above. For example, in this configuration a mount 1400 includes an air sensor 1418 in the saline channel 1412 and includes air sensor 1420 and the contrast channel 1414, instead of a bubble filter 922 in a combined saline/contrast fluid channel 940 providing extra safety detecting air in any fluid provided to catheter 1402. If the controller receives a signal from the saline air sensor 1418 or the contrast air sensor 1420, it can provide an alarm and/or align the second control valve 1428 to evacuate fluid in saline-contrast channel 1421 and upstream of saline-contrast channel 1421 via vacuum channel (flow-path) 1416. Subsequently, the saline and/or the contrast channels can be re-primed. In some embodiments, the controller may also align the first control valve 1426 to prevent contrast and saline to flow through the first control valve 1246.

FIGS. 20A-20C are schematics illustrating another example of a configuration of a contrast subsystem that can be included in a cassette, for example, instead of the contrast subsystem 1202 illustrated in FIG. 17A. In this example, the contrast subsystem is coupled to a contrast source 1108 which is located exterior to the cassette. Air sensor 1248 is positioned along contrast channel (flow-path) 1244e. A first control valve 1255a and a second control valve 1255b are three-way control valves controllable by a controller to align contrast channels 1244 for various processes. For example, first control valve 1255a and second control valve 1255b are controllable to align flow-path 1244e to flow-path 1244f and 1244h for filling contrast pump 810 with contrast through inlet port 1251, as illustrated in FIG. 20A. Second control valve 1255b can be aligned to disconnect flow-paths 1244f and 1244h, and first control valve 1255a can be aligned to connect flow-paths 1244g and 1244e for purging air from contrast pump 810 through outlet port 1253, as illustrated in FIG. 20B. As illustrated in FIG. 20C, first control valve 1255a can be aligned to disconnect flow-paths 1244e and 1244f, and disconnect flow-paths 1244e and 1244g, and second control valve 1255b can be aligned to connect flow-path 1244h and 1244i to the second tubing set (“to catheters”) to provide contrast to an associated mount.

FIGS. 20D-20E are schematics illustrating another example of a contrast subsystem that can be included in a cassette (for example, instead of the contrast subsystem illustrated in FIG. 17B). In this example, the contrast subsystem is coupled to a contrast source 1108 located exterior to the cassette and includes air sensor 1248 along the contrast flow-path 1244i. The contrast subsystem includes first control valve 1257a and second control valve 1257b which are controllable by a controller to align flow-paths 1244 for various processes. For example, first control valve 1257b can be aligned to connect contrast flow-path 1244i from contrast source 1104 to port 1253 of contrast pump 810, and second control valve 1257b can be aligned to disconnect flow-path 1244k from port 1251 for filling the contrast pump 810. Second control valve 1257b can also be aligned to disconnect flow-path 1244i from port 1253, and first control valve 1257a can be aligned to connect output port 1251 to flow-path 1444k to provide contrast to the second tubing set 1302 and to an associated mount, as illustrated in FIG. 20E.

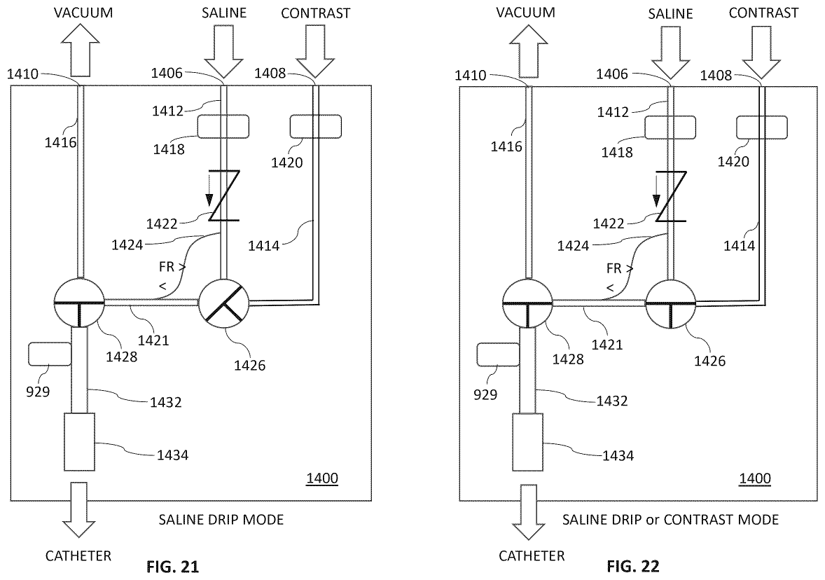

FIGS. 21-27 illustrate examples of configurations of saline, contrast, and vacuum flow-paths of mount 1400, that has components described in reference to FIG. 19, during different processes prior to or during a medical procedure using the robotic catheter system. Other configurations to provide a saline drip, contrast injection, saline flush, aspiration, and priming of mounts and the fluidic system are also possible with different components are also possible. In each of these configurations, the mount 1400 is being provided saline, contrast, and vacuum via the second tubing set 1316, the splitter 1310, the first tubing set 1302, in the cassette 1200. The various configurations allow saline, contrast and vacuum provided to the mount 1400 to be provided to connector 1434 into the lumen of a catheter coupled to, and in fluid communication with, connector 1434. In some embodiments, the connector 1434 is connected to a hub to which the catheter is coupled to, the hub including the fluid path such that saline, contrast, or vacuum provided to the hub is also provided to the lumen of the catheter. Accordingly, reference to a fluid being provided to connector 1434 also indicates the fluid is provided to the catheter in fluid communication with connector 1434. The different configurations of saline, contrast, and vacuum flow-paths are achieved by different alignments of first control valve 1426 and second control valve 1428. The controller (e.g., controller 2230, controller 2220) can control the alignment the first control valve 1426 and the second control valve 1428 based on user input from the remote control system 2210, user input from a local control system (e.g., interface 2235), predetermined instructions that the controller is configured to carry out, and/or signals from one or more sensors that provide the controller information about the system. In an example, the first control valve 1426 and second control valve 1428 can be aligned by the actuators in a drive system mount 1400 is coupled to. In some embodiments, actuators are coupled to the first control valve in the second control valve by a magnetic coupling. In other embodiments, actuators are coupled to the first and second control valves 1426, 1428 by a mechanical coupling.

FIG. 21 is a schematic illustrating an example of a configuration of the fluidic system components of a mount 1400, where valves in the mount are aligned in a “saline drip mode” fluid flow to the connector 1434, and to a catheter lumen in fluid communication with the connector 1434. A mount 1400 can be configured to be in the saline drip mode after the fluidic system, including the mount, has been primed. In operation, each mount with a catheter coupled thereto can configured to be in saline drip mode while the catheters are being moved towards and in a patient, or after another process. For example, a mount 1400 can be configured in saline drip mode after injecting contrast. In this configuration, the first control valve 1426 is aligned to prevent saline channel 1412 and contrast channel 1414 from being in fluid communication with saline-contrast channel 1421 and the second control valve 1428, except for a saline flow-path through saline restricted-flow channel 1424. The second control valve 1428 is aligned to prevent fluid communication of the vacuum channel 1416 in the connector 1434, and to allow the fluid communication of saline from saline-contrast channel 1421 to connector 1434, and to a catheter lumen in fluid communication with the connector 1434.

FIG. 22 is a schematic illustrating an example of a configuration of the fluidic system components of a mount 1400 shown in FIG. 19, where valves in the mount are aligned in a “saline drip or contrast mode” fluid flow to the connector 1434, and to a catheter lumen in fluid communication with the connector 1434. A mount 1400 can be configured to be in the “saline drip or contrast mode” after the fluidic system, including the mount, has been primed. In operation, each mount with a catheter coupled thereto can configured to be in saline drip mode while the catheters are being moved towards and in a patient, or after another process. For example, a mount 1400 can be configured in saline drip mode after injecting contrast. In this configuration, the first control valve 1426 is aligned to prevent saline channel 1412 from being in fluid communication with saline-contrast channel 1421 and the second control valve 1428, except for a saline flow-path through saline restricted-flow channel 1424, and the first control valve 1428 is also aligned to place the contrast channel 1414 in fluid communication with saline-contrast channel 1421 and the second control valve 1428. The second control valve 1428 is aligned to prevent fluid communication of the vacuum channel 1416 in the connector 1434, and to allow the fluid communication of saline from a saline drip flow-path and contrast (when injected) in saline-contrast channel 1421 with connector 1434 and to a catheter lumen in fluid communication with the connector 1434. In the configuration illustrated in FIG. 22, in some embodiments contrast can be injected and then a saline drip will continue to be provided to the connector 1434 without needing to change the alignment of the first control valve 1426 and the second control valve 1428.

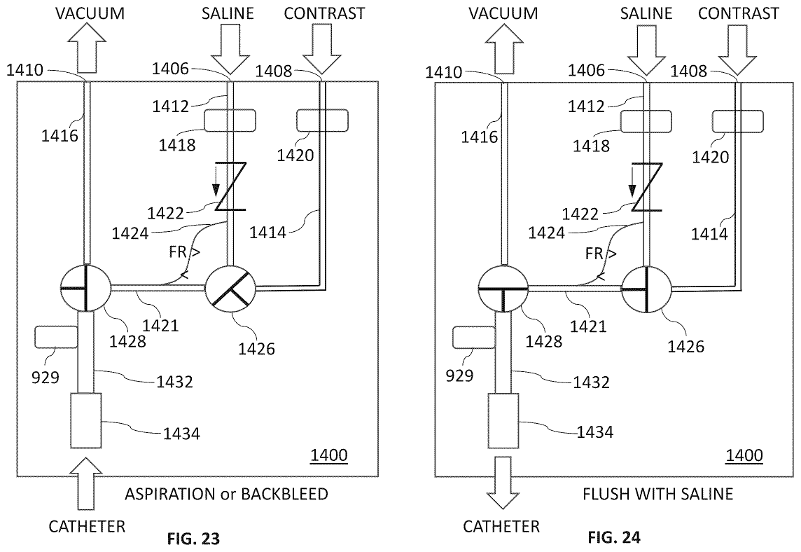

FIG. 23 is a schematic illustrating an example of a configuration of the fluidic system components of a mount 1400, where valves in the mount are aligned in a “aspiration or back-bleed mode” fluid flow to connector 1434, and to a catheter lumen in fluid communication with the connector 1434. This configuration can be used, for example, when aspirating a clot or aspirating air and fluid (e.g., blood) that may reside in a catheter in fluid communication with the connector 1434 and the portion of the flow-path between the second control valve 1428 and the catheter. In this configuration, the first control valve 1426 is aligned to prevent saline channel 1412 from being in fluid communication with saline-contrast channel 1421 and the second control valve 1428, except for a saline flow-path through saline restricted-flow channel 1424 to saline-contrast channel 1421, and the first control valve 1428 is also aligned to prevent the contrast channel 1414 from being in fluid communication with saline-contrast channel 1421 and the second control valve 1428. The second control valve 1428 is aligned to prevent fluid communication of saline or contrast from saline-contrast channel 1421 to connector 1434, and to allow fluid communication of vacuum with connector 1434 and to a catheter lumen in fluid communication with the connector 1434.

FIG. 24 is a schematic illustrating an example of a configuration of the fluidic system components of a mount 1400, where valves in the mount are aligned in a “flush with saline mode” fluid flow to the connector 1434, and to a catheter lumen in fluid communication with the connector 1434. As a nonlimiting example, a mount 1400 can be configured to be in the “flush with saline mode” after a contrast injection has been performed to clear contrast from the saline flow-path and the catheter. In this configuration, the first control valve 1426 is aligned to communicate saline from saline channel 1412 to the saline-contrast channel 1421 and second control valve 1428, and the first control valve 1426 is further aligned to prevent the contrast channel 1414 from being in fluid communication with saline-contrast channel 1421 and the second control valve 1428. The second control valve 1428 is aligned to prevent fluid communication of the vacuum channel 1416 to the connector 1434, and to allow the fluid communication of saline from a saline drip flow-path and the saline channel 1412 through saline-contrast channel 1421 with connector 1434 and to a catheter lumen in fluid communication with the connector 1434.

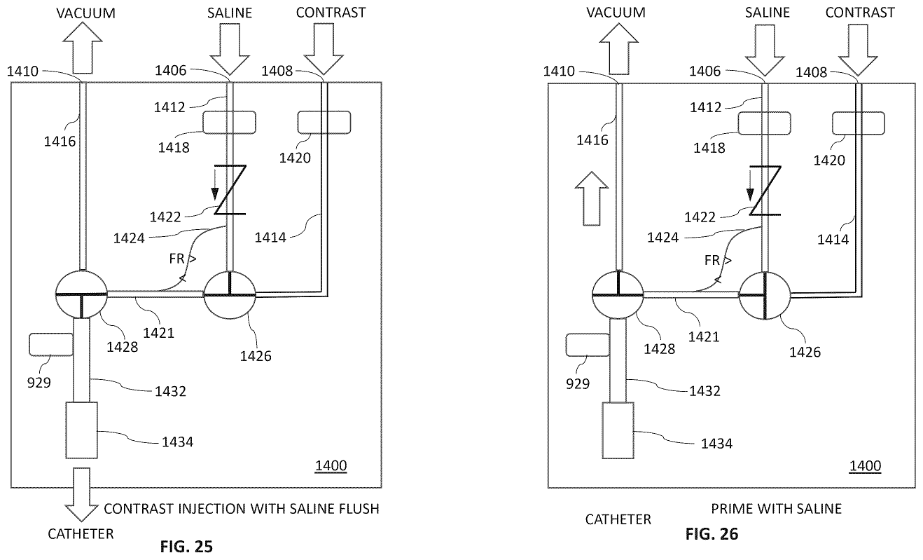

FIG. 25 is a schematic illustrating an example of the configuration of the fluidic system components shown in FIG. 19, where two three-way valves are configured in a “contrast injection with saline flush mode” to provide contrast to a catheter in fluid connection with connector 1434 and then flush the catheter with saline. In this configuration, first control valve 1426 is aligned to allow contrast channel 1414 to be in fluid communication with saline-contrast channel 1421 the second control valve 1428 and allow saline channel 1412 to be in fluid communication with saline-contrast channel 1421 and the second control valve 1428, that is, to allow saline flow to the second control valve 1428 through the saline restricted-flow channel 1424 and through the first control valve 1426 and saline-contrast channel 1421. The second control valve 1428 is aligned to provide fluid communication from saline-contrast channel 1421 that can contain saline and/or contrast to connector 1434 and to a catheter coupled to connector 1434, and prevent connection of the vacuum channel 1416 with connector 1434.

FIG. 26 is a schematic illustrating an example of a configuration of the fluidic system components shown in FIG. 19, where the control valves are configured in a “prime with saline mode” to align fluid communication of a saline channel 1412 with a relatively high saline flow (i.e., higher than a saline flow from the saline drip flow-path) to the vacuum flow-path. To prime the vacuum flow-path. In this configuration, first control valve 1426 is aligned to prevent fluid communication between the contrast channel 1414 and saline-contrast channel 1421. The second control valve 1428 is aligned to connect saline-contrast channel 1421 to the vacuum channel 1416 to provide saline to the vacuum channel 1416 for priming the vacuum channel 1416 with saline.

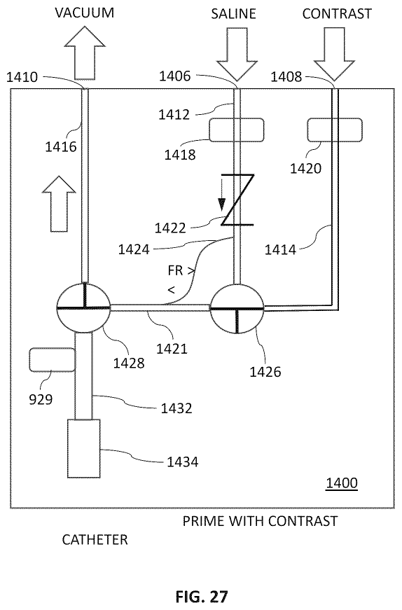

FIG. 27 is a schematic illustrating an example of the configuration of the fluidic system components shown in FIG. 19, where control valves in the mount 1400 are configured in a “prime with contrast mode” for priming the mount 1400 with contrast. In this configuration, the first control valve 1426 is aligned to connect contrast channel 1414 to saline-contrast channel 1421 to provide contrast to the second control valve 1428 and to prevent fluid communication between the saline channel 1412 and saline-contrast channel 1421, except through a saline restricted-flow channel 1424. The second control valve 1428 is aligned to connect channel 1412 to the vacuum channel 1416 and prevent fluid communication from vacuum channel 1416 and saline-contrast channel 1421 to the connector 1434. In this configuration, contrast can be provided to the mount in the contrast channel 1414 can be filled (primed) with contrast. The second control valve 1428 can connect saline-contrast channel 1421 to the vacuum channel 1416 to facilitate the flow of contrast through channel 1414 the first control valve 1426. In some embodiment, once the mount 1400 is primed with contrast, the first control valve 1426 and the second control valve 1428 can be aligned as illustrated in FIG. 21 to provide a saline drip to the connector 1434 (and catheter), or the first control valve 1426 and the second control valve 1428 can be aligned as illustrated in FIG. 22 to provide a saline drip or contrast injection to connector 1431 and the catheter coupled to the connector 1434.

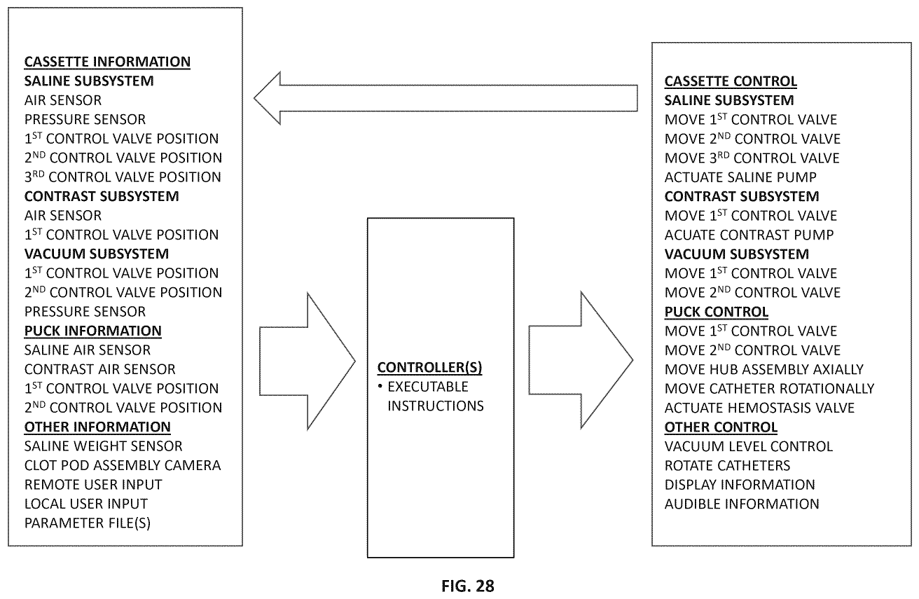

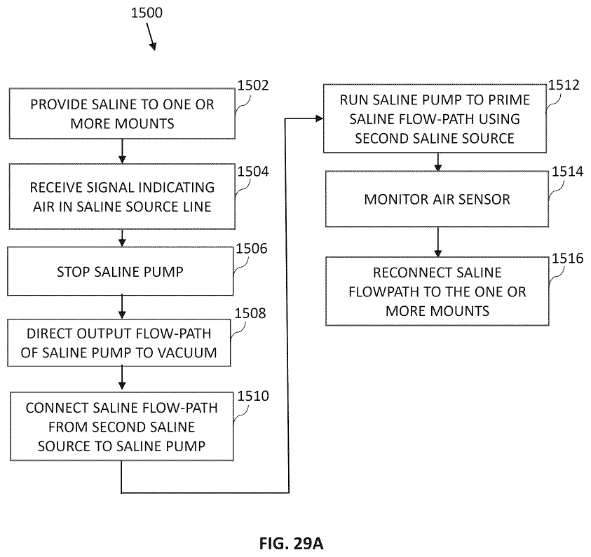

FIG. 28 illustrates examples of components of a robotic catheter system that a controller can control components (e.g., hubs, valves, pumps, etc.) to actuate and move to perform processes for a medical procedure, and examples of information that the controller can use to determine to control the components. As described in reference to FIGS. 14-27 and throughout this disclosure, information can be provided to a controller from the cassette via a communication interface, for example, the electrical interface 1236. The information from the cassette can include information from the saline subsystem air sensor, the saline subsystem pressure sensor, the contrast subsystem air sensor, the contrast subsystem pressure sensor, and the vacuum subsystem pressure sensor. If valve position sensors are included in the cassette, information from the cassette can include position information from the control valves of the saline subsystem, the contrast subsystem, and the vacuum subsystem. For example, in reference to the embodiment illustrated in FIG. 17B, position information of the first, second, third control valve of the saline subsystem, the first control valve of the contrast subsystem in the first and second control valves of the vacuum subsystem. Information from one or more mounts can be provided to the controller via the communication interface on the cassette. The information from the one or more hub assemblies (e.g., mounts and/or hubs) can include information from a saline air sensor and a contrast air sensor. If valve position sensors are included in the one or more hub assemblies, information from the one or more hub assembly can include position information of control valves in the one or more hub assemblies. For example, in reference the embodiment illustrated in FIG. 19, position information of the first and second control valves. If valve position sensors are not included in the cassette and/or the one or more hub assemblies, the controller can receive valve position information from actuators moving the valves. The controller can receive other information as well including, in some embodiments, information from a saline weight sensor(s), the drip pod camera, information and control signals from the remote medical practitioner, information control signals from a local medical practitioner, and one or more parameter files that can include specific information relating to the catheters being used, a medical procedure being performed, the medical practitioner(s) performing the procedure, and system itself.

Using the received information, and instructions (e.g., software) that the controller is configured to execute, the controller can actuate/move components of the saline subsystem, the contrast subsystem, the vacuum subsystem, and the one or more mounts. For example, in reference to the embodiment illustrated in FIG. 17B, the controller can move the first, second, third control valves of the saline subsystem, the first control valve of the contrast subsystem, the first and second control valves of the vacuum subsystem, and the first and second control valves of the one or more hub assemblies (e.g., mounts and/or hubs) to align the control valves in certain positions to communicate saline, contrast, and vacuum to fluidic connectors of the one or more hub assemblies into catheters coupled to the connectors. The controller can also actuate the contrast pump in the saline pump is necessary for a particular procedure. In addition, the controller can cause each of the one or more hub assemblies to be moved separately or together in an axial direction, and cause the one or more hub assemblies to individually rotate the catheters coupled to the hub assemblies, such that the catheters rotated separately or simultaneously. In addition, the controller can display information received, and information determines from the received information, on displays positioned locally or remotely. Also, in some embodiments, the controller can provide audible information (alarms, signals, etc.) based on the received information.

As an example of certain embodiments, the fluidics management system can comprise a plurality of catheters, one of the plurality of catheters coupled to the connector of each hub assembly (e.g., mount and/or hub) to place a lumen of the catheter in fluid communication with the connector. In some embodiments, each of the two or more hub assemblies can include a saline air sensor positioned to detect air in the third saline flow-path, and a contrast air sensor positioned to detect air in the third contrast flow-path. In some embodiments of the fluidics system, the saline air sensor and contrast air sensor are positioned in the saline and contrast third flow-paths, respectively, between the plurality of robotically actuated control valves and the second tubing set for detecting air in the saline and contrast flow-paths before it reaches the plurality of robotically actuated control valves. In some embodiments of the fluidics management system, one or more plurality of robotically actuated control valves are controlled by a control system to block the saline and contrast flow-paths to the connector based on a signal from one or both of the saline and contrast air detectors. In some embodiments, each of the two or more hub assemblies (e.g., mounts and/or hubs) includes a plurality of sensors, and wherein the first tubing set, the splitter, and each tube group of the second tubing set further comprises an electrical channel coupled to the plurality of sensors in respective hub assemblies, the electrical channel configured to communicate electrical signals from the plurality of sensors to an electrical interface on the cassette that is configured to electrically connect to a corresponding electrical interface on a pump station to provide a control system with the signals from the plurality of sensors in the mounts. In some examples, the plurality of sensors can include a saline air sensor positioned to detect air in the third saline flow-path, a contrast air sensor positioned to detect air in the third contrast flow-path, and a pressure sensor configure to sense pressure of fluid flowing through the connector. In some embodiments, the third saline flow-path in each hub assembly includes a saline restricted-flow channel the allows for a low-volume of saline flow that bypasses the first control valve that's configured to select one or both of the third saline flow-path and the contrast flow-path such that saline can be provided from the second tubing set to the connector without passing through the first control valve.

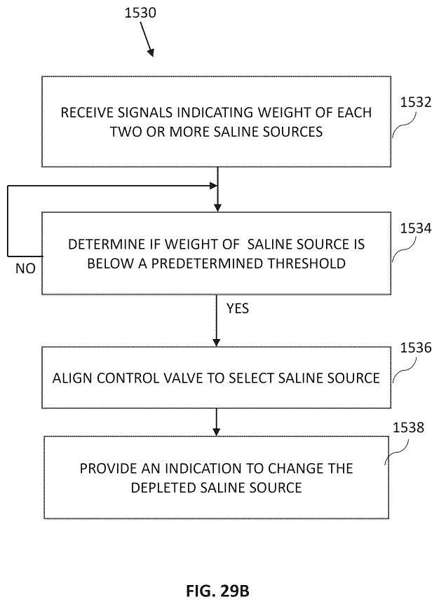

FIG. 29A is a schematic illustrating an example of a process 1500 for a controller to control providing a constant saline flow to one or more mounts (or hub assemblies). Specifically, FIG. 29A illustrates a process 1500 for monitoring saline flow and switching from a first saline source to a second saline source, and is described in reference to the embodiment of the cassette 1200 illustrated in FIG. 17B. The actuation of the pump and the valves is performed by the controller based in part on information received from an air sensor monitoring a channel providing saline from a saline source. As indicated above, the air sensor can be positioned in the cassette or in the pump station at a location to engage the saline channel providing saline from the saline source. In this example, at block 1502, the system is controlled to provide saline to one or more mounts. Saline pump 1213 is actuated for providing saline from a saline source 1106a, and the first control valve 1220 is aligned to communicate saline from saline source 1106a to the peristaltic pump 1213. The second and third control valves 1214, 1218 are aligned to communicate saline output from the saline pump 1213 to the first tubing set 1302. At block 1504, the controller receives a signal from air sensor 1205 indicating the presence of air in the saline flow-path from the saline source 1106a. Based on receiving the signal from the air sensor 1205, the controller stops the saline pump 1213 at block 1506. At block 1508 the controller directs the output of the saline pump to vacuum, for example, by aligning the second control valve 1214 to form a flow-path from the output of the saline pump to channel 1222 connected to the vacuum subsystem. In saline subsystems that are configured to use one saline source (e.g., saline bag), a user can swap the depleted saline source for another saline source. In saline subsystems that are configured to use two (or more) saline sources as illustrated in FIG. 17B, at block 1510 the first control valve 1220 can be aligned to connect a flow-path from the second saline source 1106b to the saline pump 1213. At block 1512, the controller can direct the saline pump 1213 to run such that a predetermined amount of saline passes through the saline pump 1213 (e.g., the pump is robotically controlled to run for a predetermined amount of time) and flows to the vacuum subsystem 1203 to the vacuum canister 1270, priming the saline source line from the second saline source to the second control valve 1214. While the saline pump 1213 is running, at block 1514 the controller monitors signals received from the air sensor 1205 for an indication of air in the saline flow-path. When a saline passes through the pump 1213 and air is not detected in the saline flow-path from the second saline source, at block 1514 the controller can realign the second control valve 1214 to connect the saline flow-path downstream to the first tubing set 1302 and the mounts 1400. In some processes, a user inspection of the cassette saline paths can be performed between blocks 1514 and blocks 1516.

FIG. 29B is a schematic illustrating an example of another process 1530 for a controller to control providing a constant saline flow to one or more mounts, and is described in reference to an embodiment illustrated in FIG. 17B. In some configurations, saline can be supplied to the saline subsystem 1201 by two or more saline sources 1106a, 1106b. FIG. 17B shows the first control valve 1220 aligned to provide saline from the first saline source 1106a to the saline flow-path 1206a. One or more weight sensor 1210 can support the two or more saline sources 1106a, 1106b. At the beginning of process 1530 the saline subsystem 1201 is primed and configured to receive saline from one of the two or more saline sources 1106a, 1106b. At block 1532 of process 1500, the weight sensor 1210 detects the weight of each saline source 1106a, 1106b, and the controller receives a signal indicating the weight of each saline source. At block 1534, the controller monitors the weight of each saline source based on the received signals from the weight sensor 1210, and determines if the weight of a saline source 1106a currently providing saline to the saline subsystem 1201 is below the predetermined threshold. If the weight of the saline source 1106a is not below a predetermined threshold, the controller continues to monitor the weight of the saline source. If the weight of the saline source 1106a is below a predetermined threshold indicating that the saline source 1106a is running out of saline, the process proceeds to block 1536 with the controller aligns the first control valve 1220 to place the saline flow-path 1206a in fluid communication with the second saline source 1106b. If the system has been properly primed, and the first saline source 1106a was not completely depleted, no air should be in the saline flow-path 1206 and another saline priming process should not need to be performed. If the air sensor 1205 indicates air is present in the saline flow-path 1206, the controller can perform a priming process for the saline subsystem 1201. Finally, at block 1538, the controller can provide an indication (e.g., an audible signal, a visible signal on a user interface at the remote and/or local location) for the medical practitioner to change the depleted saline source.

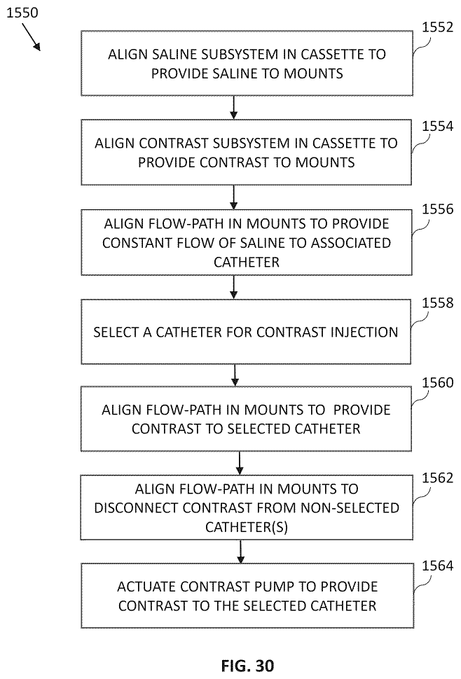

FIG. 30 is a flowchart illustrating an example of a process 1550 for providing contrast to one or more catheters. The process 1550 can be performed by a controller of a fluidics system in a robotic catheter system configured as described herein, the fluidics system being configurable to provide saline and contrast to two or more catheters coupled to mounts (e.g., hub assemblies) in various combinations. For example, to provide saline to all of the two or more catheters (for example, three catheters), or a subset of the two or more catheters (for example, catheters that are not being utilized for aspiration). The fluidics system can include, for example, a cassette 1200 as illustrated in FIG. 17B, one or more mounts 1400 as illustrated in FIG. 19, and the fluid communication channels 1300 coupled to the one or more mounts 1400 and the cassette 1200, as illustrated in FIG. 15. At block 1552 of process 1550, the controller aligns the saline subsystem 1201 in a cassette 1200 that is coupled to a pump station 2240 to provide saline to two or more mounts. In an example, FIG. 17B illustrates a configuration of the saline subsystem 1201 where the controller has aligned first control valve 1220, second control valve 1214, and third control valve 1218, and actuated peristaltic pump 1213, to provide a saline flow along flow-path 1206 to the fluid communication channels 1300.

At block 1554 of process 1550, the controller aligns the contrast subsystem 1202 in the cassette 1200 to provide contrast to two or more mounts. In an example, FIG. 17B illustrates a configuration of the contrast subsystem 1202 where the controller has aligned first control valve 1247 such that when actuated the contrast pump 810 can provide contrast to the fluid communication channels 1300. At block 1556 of process 1550, the controller aligns a flow-path in mounts to provide a constant flow of saline to a catheter coupled to the mount. FIGS. 24 and 25 illustrate two different configuration of fluidic flow-paths in a mount to provide a constant flow of saline to the catheter (e.g., a low flow rate of saline). At block 1558 of process 1550, the controller receives input that indicates a selected catheter for injecting contrast. The input can be provided, for example, from a remote controller at a remote location operated by medical practitioner. When the controller receives the selection of the catheter for a contrast injection, at block 1560 the controller aligns (if not already aligned) the contrast flow-path in the selected mount such that the contrast channel 1414 is in fluid communication with the saline-contrast channel 1421, for example, as illustrated in FIG. 22. At block 1562 of the process 1550, the controller align contrast flow-paths in non-selected mounts such that the contrast channel 1414 is not in fluid communication with a saline-contrast channel 1421. For example, in a configuration illustrated in FIG. 21. If a saline flush of the catheter is desired after contrast injection has been performed (for example, for a larger ID catheter), the controller can align flow-paths in the selected mount such that both the contrast channel 1414 and the saline channel 1412 are in fluid communication with saline-contrast channel 1421. For example, as illustrated FIG. 25. Finally at block 1564, the controller actuates the contrast pump to provide contrast through the fluid communication channels 1300 and to the selected catheter. If the mount 1400 coupled to the selected catheter is configured as illustrated in FIG. 25, after the contrast injection occurs a saline flush immediately occurs due to the connection between the saline channel 1412 and the saline-contrast channel 1421 by the first control valve 1426, to allow the full volume of saline being provided to the mount 1400 to flow through the first control valve 1426, through the saline-contrast channel 1421, through the second control valve 1428, threw primary channel 1432, and through connector 1434 to the attached catheter.

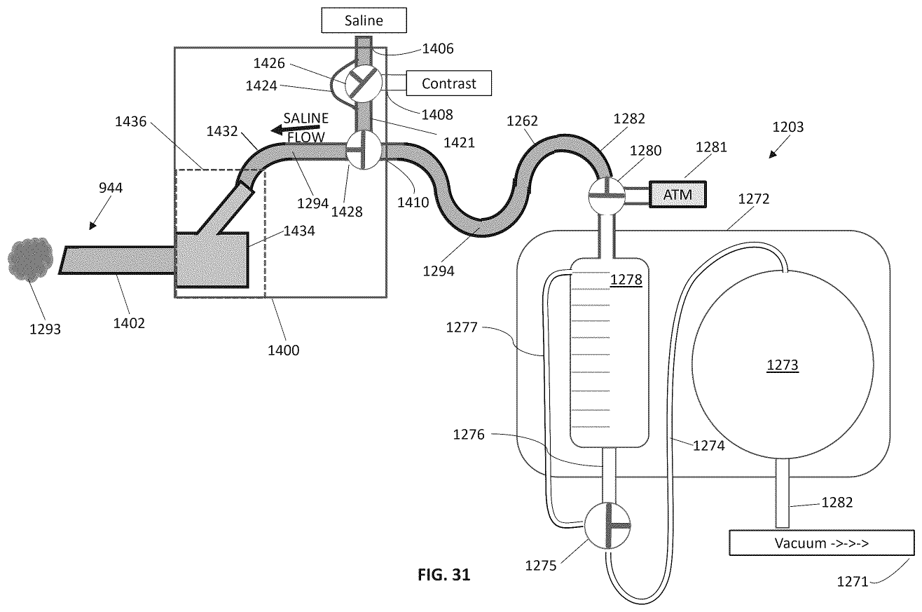

FIGS. 31-36 illustrate a representation of an example of a portion of fluidics system during processes of ingesting a clot and performing a backbleed process for a catheter. Some components may not be illustrated for clarity. Such a process can be used with fluidic system configurations disclosed herein, for example, with the vacuum subsystem 1203 illustrated in FIG. 17B and the mount configurations illustrated in FIG. 19. In FIGS. 31-36, the mount 1400 is shown as including a hemostasis valve 1434 for ease of illustration. In some embodiments, the mount 1400 can include the hemostasis valve 1434. In other embodiments, the hemostasis valve 1434 can be incorporated in a hub that mechanically and fluidically releasably couples to the mount 1400. For example, the hub can connect to the mount via a luer connector, for example, at connector 1434 (FIG. 19). In this representation, channel 1262 represents a fluid communication channel coupled to the vacuum flow-path 1282 of the vacuum subsystem 1203 and coupled to vacuum port 1410 on the mount 1400, for example, a portion of first tubing set 1302, splitter 1310, and second tubing set 1316 (FIG. 16).

In the configuration shown in FIG. 31, a low volume saline flow 1294 is being provided to the mount 1400 and catheter 1402. The configuration illustrated in FIG. 31 is an example of an alignment of control valves of the vacuum subsystem 1203 and control valves of the mount 1400 while a distal end 944 of the catheter 1402 is positioned near a clot 1293 prior to aspirating the clot 1293. In this representation, although the first control valve 1426 is aligned to prevent a high volume of saline or contrast to flow to the second control valve 1428, a small volume of saline is flowing from the saline subsystem through a saline restricted-flow channel 1424 (shown in FIG. 19, not shown in FIGS. 31-36,) to second control valve 1428 and downstream through primary channel 1432 to the mount 1400 and the catheter 1402. In an alternative configuration, the first control valve 1426 and the second control valve can be aligned as illustrated in FIG. 22 in a saline drip or contrast mode. The drip pod 1272 is receiving vacuum from a vacuum source (e.g., vacuum canister 1270, vacuum regulator 1269, and vacuum pump 1267 FIG. 17B). The first control valve 1275 of the vacuum subsystem is aligned such that the vacuum is provided to the drip chamber 1278. The second control valve 1280 of the vacuum subsystem is aligned to stop the vacuum flow-path downstream of the drip pod 1272, such that the vacuum flow-path 1282 between the second control valve 1280 and the second control valve 1428 may contain saline 1294.

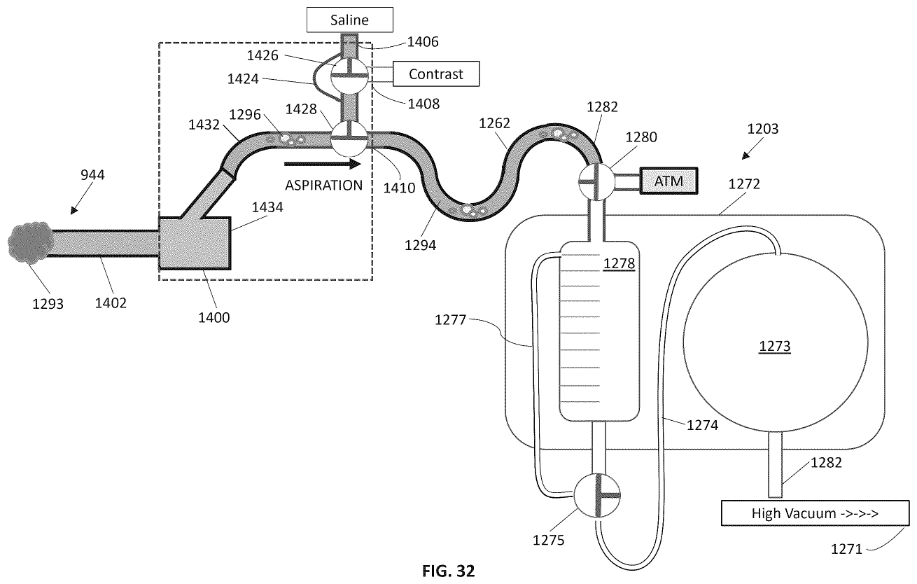

FIG. 32 illustrates an example of the portion of the fluidics system represented in FIG. 31 where “high” vacuum is provided to the catheter 1402 to aspirate a clot when the distal end 944 of the catheter 1402 is in contact with the clot 1293 such that the clot can be ingested and captured in the clot pod 1273. In this configuration, the vacuum subsystem first control valve 1275 and second control valve 1280 are aligned to provide a vacuum flow-path 1282 through the drip pod 1272 to the mount second control valve 1428 such that high vacuum is applied to the mounts 1400 in the catheter 1402. During aspiration, a controller can control the vacuum regulator to provide a high vacuum flow-path to the mount 1400 which causes air bubbles 1296 to form in the vacuum flow-path between the mount 1400 and the drip pod 1272, and in the catheter 1402 and in the mount 1400.

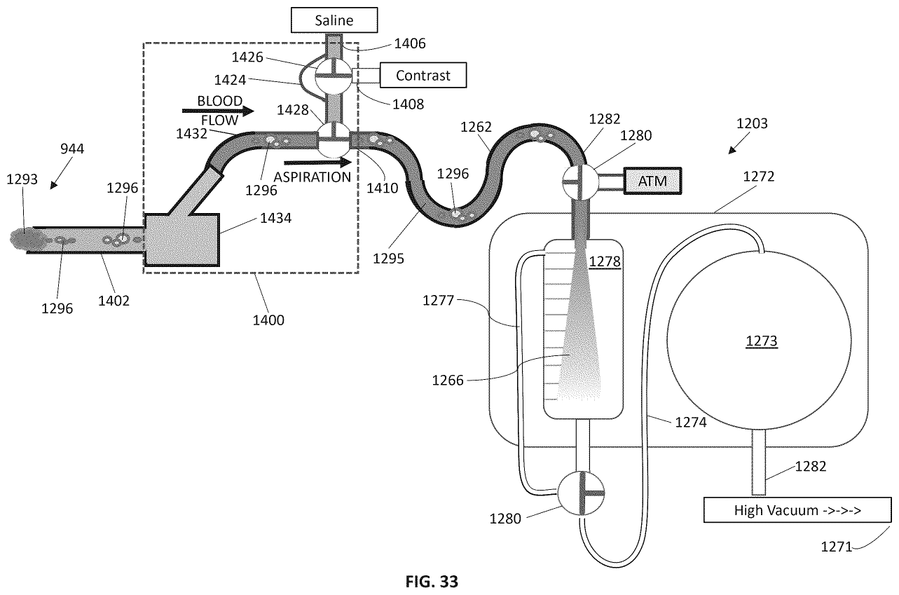

FIG. 33 illustrates same configuration of a fluidics system as illustrated in FIG. 32 where “high” vacuum is provided to a catheter 1402 at slightly later time than the example illustrated in FIG. 32. In the representation illustrated in FIG. 33, the clot 1293 has at least partially into the catheter 1402 and the vacuum flow-path from the catheter 1402 to the trip chamber 1278 causes the clot 1293 and blood from the patient to be ingested by the catheter 1402 and flow through the mount second control valve 1428 to the drip pod 1272 blood enters the chamber 1278 and a high flow rate 1266. The high vacuum causes air bubbles 1296 to be formed in the aspirated blood flow and the catheter 1402.

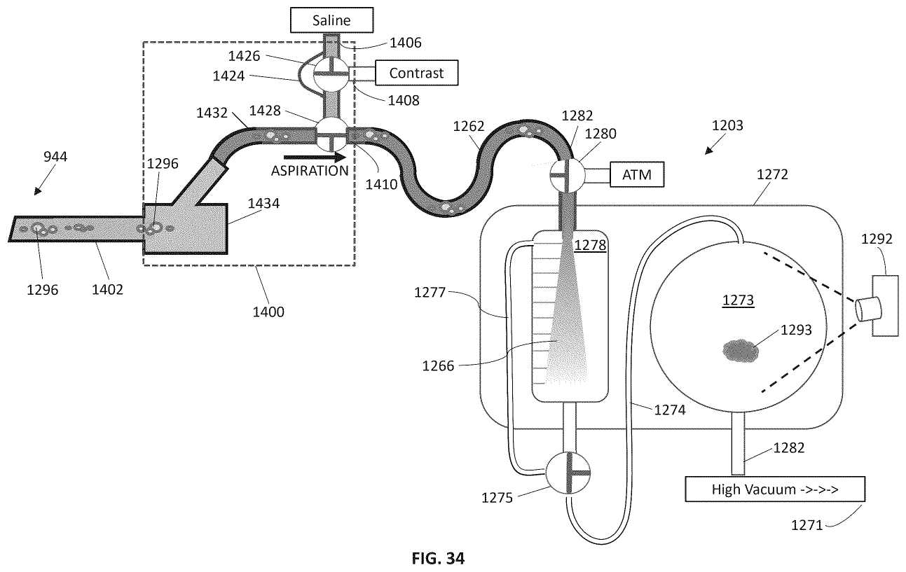

FIG. 34 illustrates same configuration of a fluidics system as illustrated in FIG. 32 where “high” vacuum is still provided to a catheter 1402 but FIG. 34 represents a later time than the example illustrated in FIG. 33. In the representation illustrated in FIG. 34, high vacuum is still being applied to the vacuum flow-path, the clot 1293 is ingested is captured in the clot pod 1273. When the control valves are aligned to provide a high vacuum flow-path to the catheter, air bubbles 1296 are formed in the stream of blood flowing back from the catheter distal tip to the drip pod 1272. An imaging sensor (“imaging system”) 1292 can be positioned to generate an image of the clot pod 1273, and the image can be displayed locally and/or displayed at the remote location for viewing by a medical practitioner. The imaging system 1292 can include a single imaging device, or multiple imaging devices to image different portions of the fluidic system at the same time. The imaging system 1292 can also be positioned to generate an image of the drip chamber 1278, and/or the drip chamber 1278 and the clot pod 1273. This can be advantageous for performing a method of remotely controlling a robotic catheter system to aspirate material, wherein the robotic catheter system providing one or more liquids and vacuum from a cassette releasably coupled to a pump station to a plurality of catheters, each catheter coupled to one of a plurality of hub assemblies in fluid communication with the cassette. Such methods can include providing vacuum to a selected one or more catheters of the plurality of catheters, communicating material through the selected one or more catheters through a vacuum flow-path into a drip chamber, capturing images depicting material in a drip chamber through a transparent wall of the drip chamber with an imaging system; and communicating the captured images to a remotely located control system for displaying the fluid level of the drip chamber. In some embodiments, such a method can further include receiving a control signal from the remotely located control system at a local control system to stop communicating material through the selected one or more catheters and through the vacuum flow-path into the drip chamber. As described hereinbelow, the drip chamber can include a first portion and a second portion, the second portion being shallower than the first portion and the second portion, and further can include graduations positioned over the second portion to indicate a fluid level in the drip chamber. Such methods can include capturing images depicting material collected in a clot pot through a transparent wall of the clot pod with the imaging system, and communicating the captured images to the remotely located control system for displaying the material collected in the clot pod. Such methods can include, after communicating the captured images to the remotely located control system, receiving at a local system controller, from the remotely located control system, a control signal to control a clot aspiration procedure performed by the robotic catheter system. Such methods can further include capturing images depicting material in the clot pod, and communicating the captured images depicting material in the clot pod to a remotely located control system for displaying the material collected in the clot pod. Capturing images of the drip chamber and the clot pod can include capturing images of the drip chamber and the clot pod by an imaging system having a single imaging device. Capturing images of the drip chamber and the clot pod can include capturing images of the drip chamber and the clot pod by an imaging system using two imaging devices.

In some embodiments, the generated image can be processed by the controller using feature detection functionality (e.g., image processing) to assess the presence of a clot in the image, and/or size or characteristics of the clot or clot material, and this information can be displayed locally or at the remote location. However, at this portion of the process the clot pod may include a mix of blood, air, and clot material in the clot pod and it can be hard to determine if the clot is in the clot pod until a saline flush is performed. In some embodiments, the clot is assessed and the controller uses the assessed information to determine one or more subsequent actions, For example, to notify a medical practitioner of the presence of the clot in the clot pod 1273, to start another process or process step (e.g., begin to perform a saline flush).

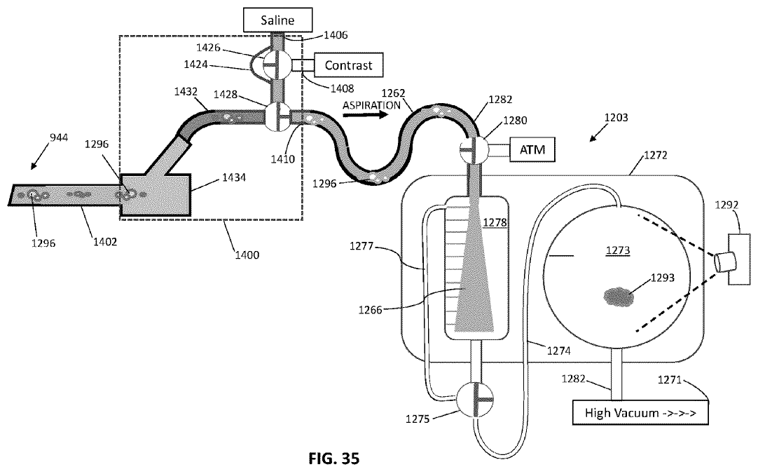

FIG. 35 illustrates the fluidics system shown in FIG. 34 where the controller is controlling the vacuum regulator to provide “high” vacuum in the vacuum flow-path 1282 to facilitate a saline flush of vacuum channel 1262 and a saline flush of the drip pod 1272, including the clot pod 1273. Saline flow during a “saline flush” is a relatively high volume of saline compared to the low volume of saline flowing through a saline restricted-flow channel 1424 in the mount in a normal operating configuration. To perform a saline flush, the controller can receive an input to perform a saline flush, for example, from a remote control operated be a medical practitioner at the remote location, or from a process being run by the controller (e.g., computer executable instructions). In some examples, a medical practitioner sees sufficient flow of fluid through the drip chamber and then the physician turns off aspiration, and the system can flush the clot pod either automatically or as controlled by an input of a medical practitioner. In some examples, signals from the pressure sensors 1279, 929 in the cassette and mount can used by a controller to detect a detect high-flow condition and the controller can automatically turn off aspiration by actuating a control valve in the mount or the vacuum subsystem.

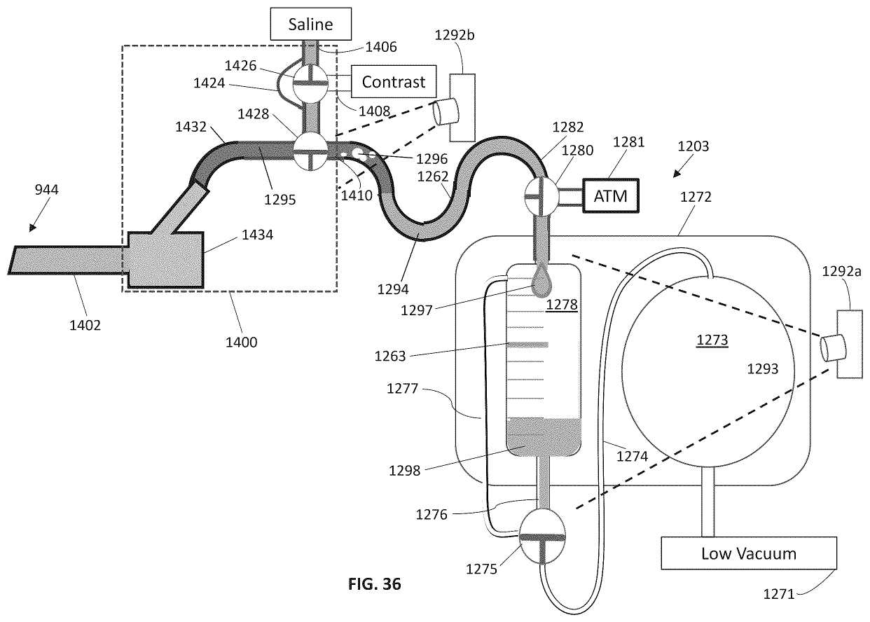

In some examples, the imaging sensor 1292 generates an image of the clot pod 1273, the controller processes the image using image processing (e.g., feature detection), a signal is generated to indicate a clot has been captured in the clot pod 1273, and the controller begins a saline flush process based at least in part on such a signal. When a controller receives the signal to perform the saline flush, if not already so aligned, the controller can align the vacuum subsystem first control valve 1275 to provide vacuum through the drip chamber 1278, align the first control valve 1426 of the mount 1400 to provide a saline flow-path through the first control valve 1426 to the second control valve 1428, and align the second control valve 1428 to provide a high volume saline flow-path through the second control valve 1428 and channel 1262 to the second control valve 1280 of the vacuum subsystem. The controller can further align the second control valve 1280 to allow a saline into the drip pod 1272. In the example illustrated in FIG. 35, a relatively high volume of saline flows through channel 1282, into the drip chamber 1278, and flows through the vacuum subsystem first control valve 1275 and the clot pod 1273, flushing blood and any clot material from the drip chamber 1278 into the clot pod 1273, and flushing blood from the clot pod 1273 out through vacuum port 1271 to a vacuum canister such that the clot 1293 is more easily visible for viewing and assessment by a medical practitioner and/or an imaging sensor 1292. While the saline flush is occurring, fluid (e.g., blood, saline) in the vacuum flow-path 1282 will likely contain air bubbles due to the high vacuum, which necessitates a backbleed process (e.g., described in reference to FIG. 36) to remove the air filled blood from the system and prevent it from flowing back into the patient.

FIG. 36 illustrates an example configuration of the fluidics system shown in FIG. 35 during a “backbleed” process. In the representation shown in FIG. 36, channel 1262 represents the vacuum communication channel between the vacuum subsystem 1203 in the mounts 1400. During backbleed, a “low” level vacuum is provided to the vacuum channel 1262, mount 1400, and catheter 1402 to cause a slow flow of blood foam and fluid containing air from the catheter and mount into the vacuum channel 1262 towards the vacuum subsystem 1203, removing blood foam from the catheter and mount. The blood foam is moved out of the mount and at least past the mount second control valve 1428 of the mount and into the vacuum channel 1282 so that subsequent fluid provided to the catheter will not include any blood foam. A backbleed process can be performed to clear blood foam from the catheter and/or the mount, and is typically performed after aspiration or whenever a high level of vacuum has been applied to the catheter such that there is a likelihood of blood foam in the catheter or the mount. In some embodiments, prior to starting the backbleed process, the controller can align the second control valve 1280 to place the drip chamber 1278, which is subjected to vacuum, in fluid communication with a connector 1281 open to atmospheric pressure, and align the first control valve 1275 to align channel 1276 to the clot pod inlet 1274 to quickly flush out the drip chamber and the clot pod 1273 with air, and then once it has been flushed the controller can align the second control valve 1280 to close the connection to atmospheric pressure and align the first control valve 1275 to place the clot pod inlet 1274 in fluid communication with drip chamber bypass channel 1277. In some embodiments, a filter can be coupled to the connector (e.g., inside the connector, or inline with the connector), the filter structured to filter liquid (e.g., blood, saline, contrast) and prevent liquid from being exhausted out of the system when the second control valve 1280 is actuated to place the drip chamber 1278 in fluid communication with the atmosphere via the connector. Such a filter can be advantageous to prevent unwanted spray of liquid from the drip chamber (or the vacuum line) if there is pressure in the drip chamber that is higher than atmospheric pressure which can cause liquid in the drip chamber to be exhausted through the connector 1281. The controller can receive a signal indicating to begin a backbleed process, the signal based on an input provided by medical practitioner at a remote location or at the locally. In some embodiments, the signal may be based on another process controlled by the controller, for example, that a saline flush process has been completed. In some embodiments, the controller may provide a visual or audio indication that a backbleed process may be necessary, for example, if a pressure sensor 929 (FIG. 19) in the mount indicates a vacuum level sufficient to cause blood foam to occur. Similar to the actions performed in FIGS. 31-35, the controller aligns control valves in the mount and the vacuum subsystem, and controls the level of vacuum provided by the vacuum subsystem to perform the backbleed process.

In an example, prior to starting the backbleed process the control valves in the vacuum subsystem 1203 and the mount 1400 may be aligned as illustrated in FIG. 35 with blood foam present in the catheter 1402 and the mount 1400. When the controller receives a signal to begin backbleed, the vacuum source is controlled to provide low vacuum through the vacuum subsystem 1203, and the first control valve 1426 of the mount is aligned to prevent flow of saline or contrast to the second control valve 1428 (note: a small amount of saline may still be flowing towards the second control valve 1428 via the saline restricted-flow channel 1424). The second control valve 1428 is aligned to place the catheter 1402 and primary channel 1432 in fluid communication with the vacuum channel 1262. In the vacuum subsystem, first control valve 1275 is aligned to provide vacuum to the drip chamber 1278 via bypass channel 1277 and stopping fluid flow from the clot pod inlet line 1274 to the drip chamber outlet line 1276, and the second control valve 1280 is aligned (or maintains its alignment) to provide fluid flow from channel 1262 to the drip chamber 1278. This alignment of the control valves with the low vacuum level causes blood foam other fluid to slowly flow from the catheter 1402 in the mount 1400 through the park second control valve 1428 and into channel 1262 towards the drip chamber 1278, and drip into the drip chamber 1278. The level of vacuum is set to be a low level that causes this slow fluid flow to occur such that the fluid can be visually seen dripping into and filling the drip chamber 1278, such that fluid 1298 accumulates in the bottom of the drip chamber 1278. One or more graduations (lines) 1263 along the side of the drip chamber 1278 allow a medical practitioner to visually determine how much fluid has been back-bled from the catheter/mount. As shown in FIG. 36, channel 1262 between the vacuum subsystem 1203 in the mount 1400 contains saline at the beginning of the backbleed process (for example, if being done after saline flush). During the backbleed process, saline in channel 1262 slowly flows to and into the drip chamber 1278, and the drip chamber 1278 is configured to be visible so a user can see it filling with fluid. Blood foam and other fluid in the mount and the catheter flows from the catheter lumen and from primary channel 1432 through the second control valve 1428, out of the mount 1400 into channel 1262 and flows towards the drip chamber 1278, while saline in the channel 1262 upstream of the blood foam 1296 flows into the drip chamber 1278.

Channel 1262 represents the vacuum communication in the first tubing set 1302, the splitter 1310, and the second tubing set 1316. In some embodiments, the total length of channel 1262 is about 14 feet which is longer than a total length of any fluid flow-path in the mount 1400 and the catheter 1402, for example, between two and four times longer. Correspondingly, the volume of fluid (blood foam, saline) in channel 1262 is also two to four times the volume of blood foam and fluid in the catheter 1402 and the mount 1400. Accordingly, removing blood foam from the catheter and mount during a backbleed process does not require all of the blood foam flow through the entire channel 1262 to reach the drip chamber 1278. Instead, the blood foam need only to be removed from the catheter 1402 and the mount 1400 and flow into the channel 1262. FIG. 36 illustrates this fluid flow, showing blood foam 1296, that has been removed from the catheter 1402 and the mount 1400 resides in distal portion of channel 1262 while a proximal portion of the channel 1262 contain saline which is dripping into the drip chamber 1278. Blood 1295 in primary channel 1432 of the mount is free of blood foam. Thus, to minimize the loss of blood from the patient, only a predetermined amount of fluid needs to be back-bled to remove blood foam from the mounts 1400 and the catheter 1402, the predetermined amount of fluid being dependent at least in part on volume of liquid that can be in the mounts 1400 and the catheter 1402, which is determined based on the size of the catheter 1402 and the size of primary channel 1432. In an example, if the volume of the catheter 1402 and the primary channel 1432 is 5 mL, to ensure blood foam has been sufficiently removed from the catheter and mount, a fluid volume of greater than 5 mL (e.g., at least about 6 mL, 7 mL, 8 mL, 9 mL, or 10 mL) should flow into the drip chamber 1278 during backbleed. The amount of blood flowing into the drip chamber 1278 can be monitored using the graduation lines 1263, and when the fluid level reaches a certain amount (e.g., 10 mL) the vacuum subsystem first control valve 1275 can be aligned to connect the drip chamber outlet line 1276 to the clot pod inlet line 1276, allowing fluid collected in the drip chamber 1278 to be flushed to the vacuum canister. In an example, backbleed is performed to fill the drip chamber 1278 with about 10 mL of fluid, the fluid in the drip chamber 1278 is flushed to the vacuum canister, and the first control valve 1275 is realigned such that the vacuum flow-path is from the clot pod inlet line 1274 to the bypass line 1277. Then, a second volume of 10 mL of fluid is allowed to fill the drip chamber 1278, the first control valve 1275 is aligned to connect the drip chamber outlet line 1276 to the clot pod inlet line 1276, the fluid in the drip chamber 1278 is again flushed to the vacuum canister.

In some embodiments, an imaging sensor 1292 can be positioned to generate an image of the drip chamber 1278 and the image can be displayed on user interfaces locally and at the remote location such that the flow of fluid (e.g., saline) dripping into the drip chamber can be visually seen by a medical practitioner to verify backbleed is occurring and determine when to flush out the fluid in the drip chamber. Images generated of the drip chamber can be displayed in various ways on user interfaces on a local display or a remote display to help determine the amount of back-bled fluid. In an example, an image of the drip chamber can be displayed and a target volume mark can be shown at a certain level on the drip chamber (e.g., as an overlay) to help a viewer determine when enough fluid has been back-bled. In an example, placement of the target volume mark can be at a predetermined level. In an example, placement of the target mark can be at a level based in part on characteristics of the system, for example, which catheters are used, lengths of certain fluid channels in the hub assemblies, and the like. The target mark can be displayed in a color unique in the displayed image to be more visible to a user. In some embodiments, image processing can be performed on images of the drip chamber to determine quantitatively the volume of the back-bled fluid, and the determined amount can be shown on a user interface on a local display or a remotely located display.

The amount of fluid that is needed to be back-bled is dependent on the effective ID of the catheter 1402 (which may be affected if another elongated device is positioned in the lumen of the catheter) and the volume of the primary channel 1432. In some embodiments, this information can be predetermined and stored in a parameter file, and used by the controller determine how much backbleed is needed and indicate the amount or status to a medical practitioner at the remote location or locally. In some embodiments, the controller can perform the backbleed process automatically (while being monitored by a medical practitioner based at least in part on predetermined information about the size of the catheters being used. In one example, the predetermined information assumes that there are no other elongated devices in the catheter lumen, such that the estimated volume of the catheter is its maximum volume.

During a backbleed process, in typical embodiments, channel 1262 is transparent and the fluid flow out of the mount vacuum port 1410 through channel 1262 can be visually monitored by a medical practitioner locally. When blood exiting the vacuum port 1410 no longer includes blood foam, the backbleed process can be stopped, for example, by providing an input to the controller to end the backbleed process. If additional procedures are required, the controller can configure the system for such processes (e.g., saline drip and contrast mode). In some embodiments, an imaging sensor 1292b can be positioned to generate an image of a portion of channel 1262 proximate to the mount vacuum port 1410. For example, the portion of the second tubing set 1316 coupled to mount 1400 that includes the vacuum subchannel 1335, or another portion of the first tubing set or the second tubing set, preferably a portion of vacuum tubing close to the mount. In some embodiments, the system can include two or more imaging sensors to generate images of the drip chamber, the clot pod, and/or a portion of tubing near the mount (e.g., to see whether blood back-bled from the mount includes blood foam). The image can be displayed locally and/or provided to the remote location for medical practitioner performing the procedure can see the characteristics of the blood flowing out of the mount to determine if it contains blood foam. In this embodiment, the imaging sensor 1292 can be positioned as a suitable place in the local operating environment.

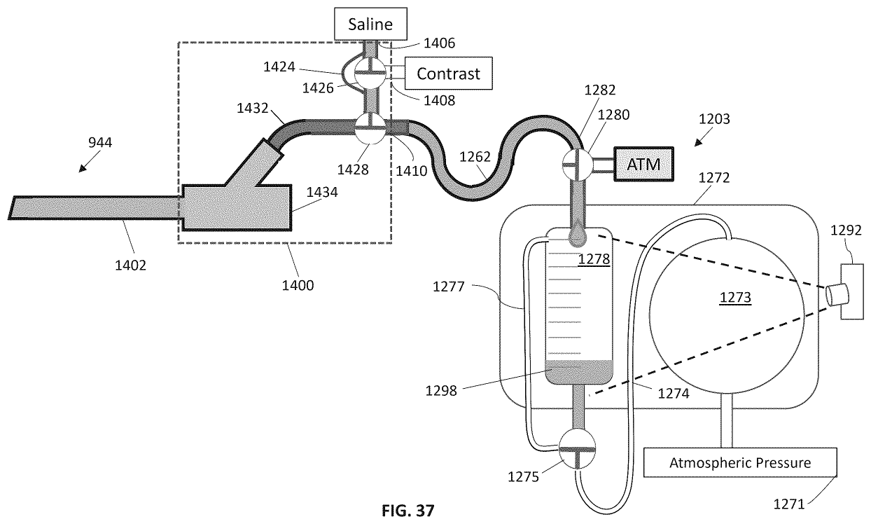

FIG. 37 illustrates a representation of an example configuration of a fluidics system for performing a patency check of a catheter. In this example, a controller can receive an input to perform a patency check of a selected one of the one or more catheters of the system, for example, guide catheter 1402a (FIG. 19), procedure catheter 1420b, or access catheter 1402c.A patency check can be performed with no other elongated device in the lumen of the catheter, or the patency check can be performed with an elongated device in the lumen of the catheter. In this example, the controller aligns the mount first control valve 1426 to prevent saline or contrast flow to the second control valve 1428, except for saline flowing in a saline restricted-flow channel 1424 (FIG. 19) and aligns the second control valve 1428 to put primary channel 1432 of the mount 1400 in fluid communication with vacuum channel 1262. The controller also aligns the vacuum subsystem 1203 first control valve 1280 to allow fluid flow from channel 1262 into the drip chamber 1278, aligns the second control valve 1275 such that the clot pod inlet line 1274 is in fluid communication with the bypass line 1277, and controls a vacuum source/vacuum regulator to set a vacuum level provided to the vacuum subsystem 1203 at about atmospheric pressure, or at a low vacuum level. Because the channel 1262 contain saline prior to the patency check being performed, in this configuration if the catheter is “clear” then saline should drip into the drip chamber 1278 at a rate that can be predetermined, for example, based on the size of the specific catheter used for the patency check, the level of vacuum, and other system parameters.

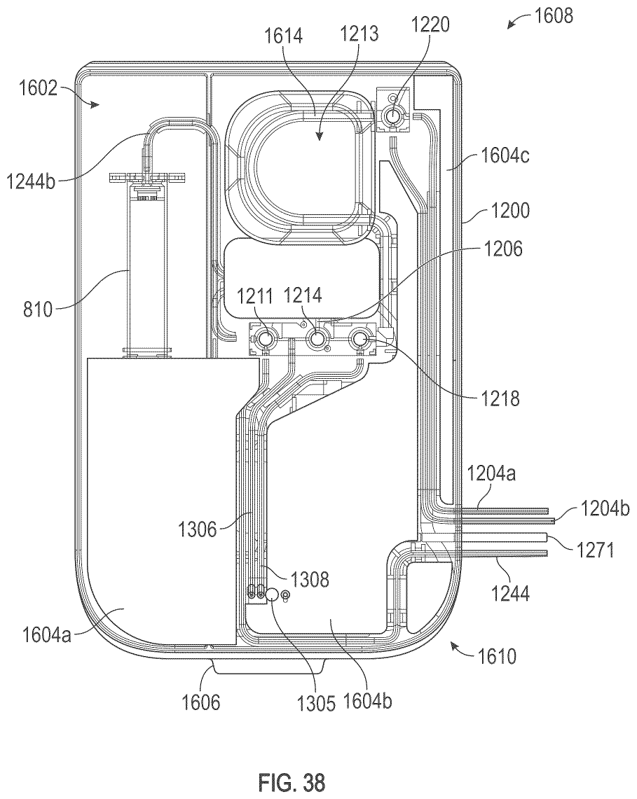

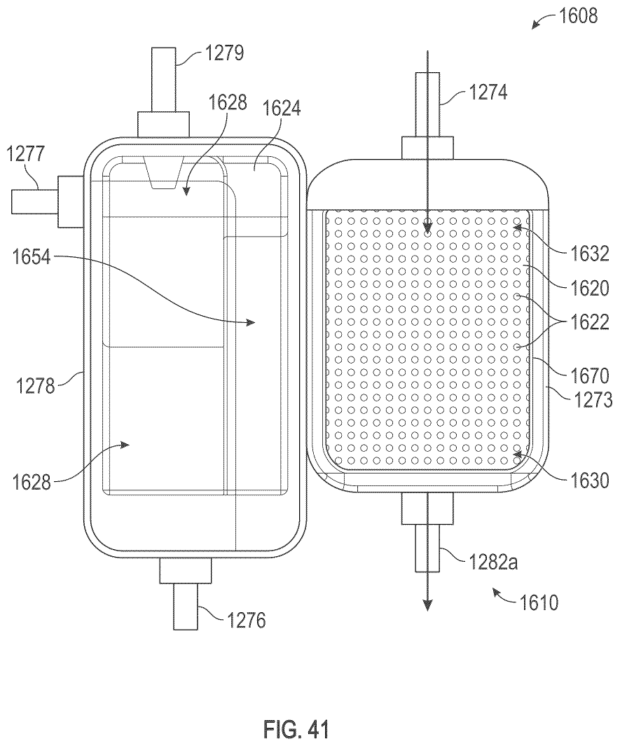

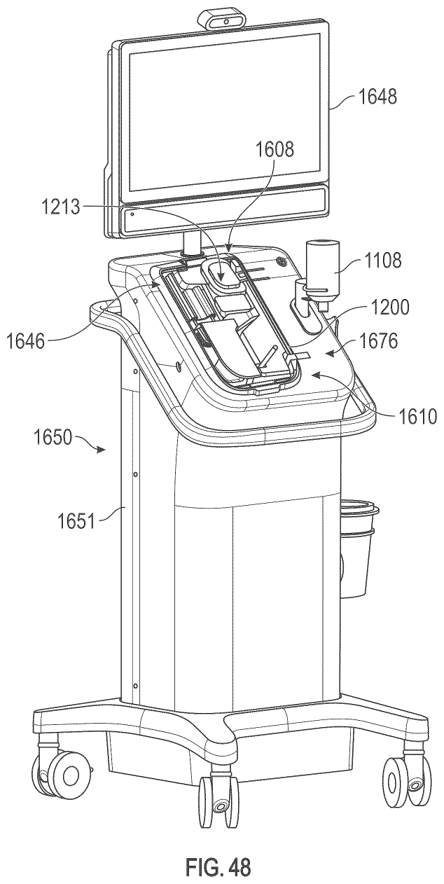

FIGS. 38-60 illustrate examples of a cassette (e.g., FIGS. 38-40), a drip chamber and clot pod (e.g., FIGS. 41-47), and a pump station and certain components of an interface of the pump station (e.g., FIGS. 48-60) that couple to and interacts with components of the cassette for providing saline, contrast, and vacuum (aspiration) to one or more catheters, each catheter being coupled to a hub assembly that can be moved by the robotic catheter system to perform a surgical procedure. Interface components can include, but are not limited to, actuators that couple to and move control valves in the cassette, a portion of a peristaltic pump that interacts with a flexible saline channel in the cassette, a syringe actuator that couples to and operates a contrast syringe, air bubble sensors that are positioned to sense air in a saline channel and a contrast channel in the cassette, and an electrical interface that electrically and mechanically couples to a corresponding electrical interface on the cassette, and are further described in reference to FIGS. 48-50.

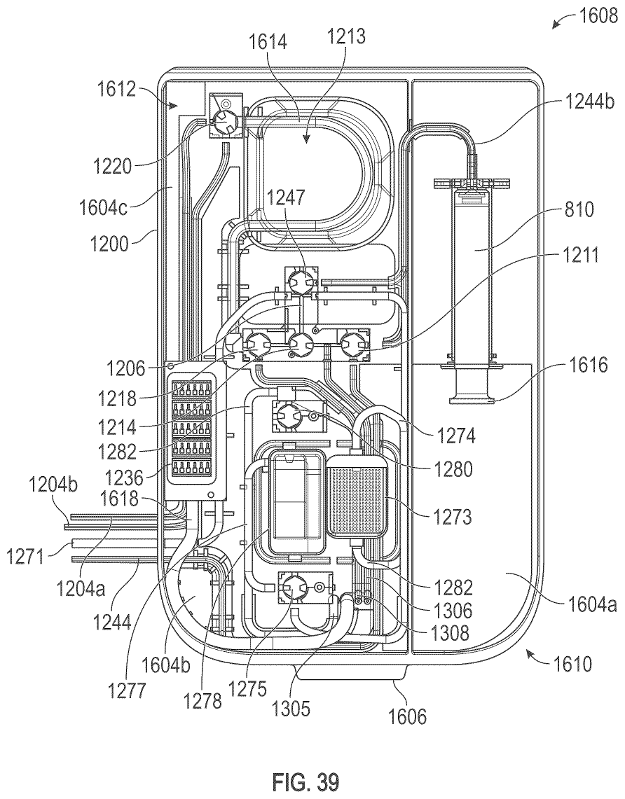

FIG. 38 shows an elevation view illustrating a distal side 1602 of an example of a fluidic cassette (or “cassette”) 1200 that is configured to be removably couplable to a pump station. For example, the exemplary pump station 1650 illustrated in FIG. 48. The fluidic cassette 1200 may also be referred to as a fluidic cartridge (or “cartridge”). In this embodiment, when the cassette 1200 is coupled to a pump station, the distal side 1602 is positioned facing away from the pump station, the proximal side 1612 (FIG. 39) is positioned facing the pump station and is coupled to the interface of the pump station (see for example, FIGS. 48 and 51-54) , and the cassette is oriented generally as shown in FIG. 38, with the upper portion 1608 of the cassette positioned higher on the pump station than the lower end 1610. The saline source(s) and the contrast source are positioned above the cassette 1200 (i.e., higher vertically) to allow saline and contrast to flow into the cassette using gravity. The saline and contrast flow through their respective flow paths in the cassette 1200 by pressure generated by the contrast pump (syringe) 810 and the saline peristaltic pump 1213. The embodiments illustrated in FIGS. 38-60 provide numerous features and advantages. For example, these embodiments allow sterile placement of the cassette on the pump station, that is, allowing a sterile technician to place the cassette on the pump station without breaching sterility. Also, these embodiments allow engagement of the cassette with less than 20 Newtons of force. These embodiments also allow retention of the cassette on the pump station when actuators of the pump station are not engaged with components of the cassette 1200. Further, these embodiments allow for the cassette 1200 to be easily removed from the pump station without the use of tools.

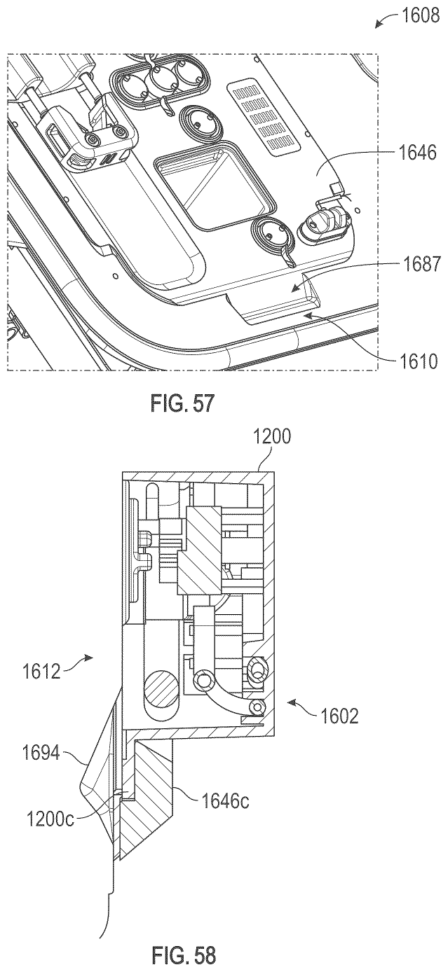

The example cassette 1200 in FIG. 38 can include fluidic components illustrated in FIGS. 17A and 17B (which may be indicated by a common reference number), or other components. This configuration shows the cassette including a contrast syringe 810, the contrast channel 1244b, a contrast refill stopcock 1211, the saline prime stopcock 1214, saline bypass stopcock 1218. This embodiment also includes a control valve 1220 which is used to select receiving saline from one of two saline sources. In other embodiments, a connector can be used, instead of control valve 1220, to connect multiple saline sources to the saline flow path in the cassette. In this embodiment, the cassette includes a portion of a peristaltic pump 1213 (e.g., tubing that is part of the saline channel 1614) and an aperture (or a recess) which is configured to receive a portion of the peristaltic pump from the pump station interface when the cassette is coupled to the pump station, the received portion of the peristaltic pump interacting with the tubing positioned within the recessed area. In this example, the cassette 1200 has proximal and distal surfaces that are transparent to allow for a user to see the components in the cassette 1200, which can be advantageous to inspect fluid channels or positions of valves, or position of a contrast pump. In this example, the cassette also include masking 1604a-c which obstructs certain components of the cassette from view when viewed from the distal side 1602, for example, portions of the saline, contrast, and vacuum channels, the drip chamber and the clot pod, and the electrical interface. The masking 1604 helps to provide a less complicated looking cassette for a user, while allowing certain portions to still be easily inspected by a user, for example, certain portions of the contrast and saline channels. In other embodiments, the cassette 1200 may include different masking configurations or no masking. The cassette 1200 including certain fluidic components for providing saline, contrast, and vacuum to one or more hubs that can be included in a robotic catheter system. Similar to the cassette illustrated in FIG. 17B, this example cassette includes a saline channel 1308, contrast channel 1306, and a vacuum channel 1305 which provides saline, contrast, and vacuum to one or more catheters of the robotic catheter system. In this embodiment, channels 1204a and 1204b are configured to be coupled to saline sources that provide saline into the cassette; channel 1244 is configured to be coupled to the contrast source that provides contrast into the cassette, and channel 1271 is configured to be coupled to a vacuum source (e.g., vacuum cannister 1270, FIG. 17B) to provide vacuum to the cassette. The cassette 1200 also includes a lower connector 1606 having a portion that extends away from proximal side of the cassette into a recess 1687 (FIG. 57) of the pump station, as illustrated in FIG. 58.

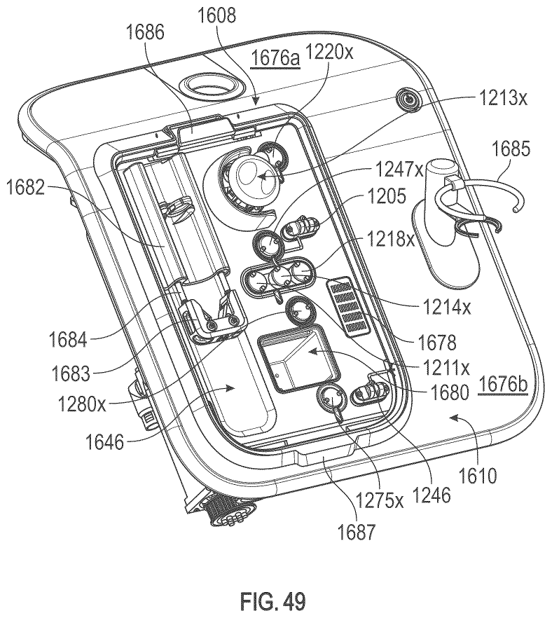

FIG. 39 is an elevation view illustrating the proximal side 1612 of an embodiment of the cassette 1200 that is shown in FIG. 38. Components in the cassette 1200 that are also illustrated in FIG. 17B have the same reference number. More clearly visible in FIG. 39, the contrast pump 810 is oriented such that a proximal end is positioned towards the lower end 1610 of the cassette 1200 when the cassette 1200 is coupled to the pump station. In this example, the contrast pump 810 can be glued onto plastic tabs in the cassette to hold it in place. When the contrast pump 810 is receiving contrast, the proximal end 1616 is moved towards the lower end 1610 of the cassette. When the contrast pump 810 is actuated to provide contrast, the proximal end 1616 is moved upward towards the upper end 1608 of the cassette 1200. FIGS. 49-51 illustrate the contrast pump 810 being coupled to the contrast pump actuator in the pump station interface.

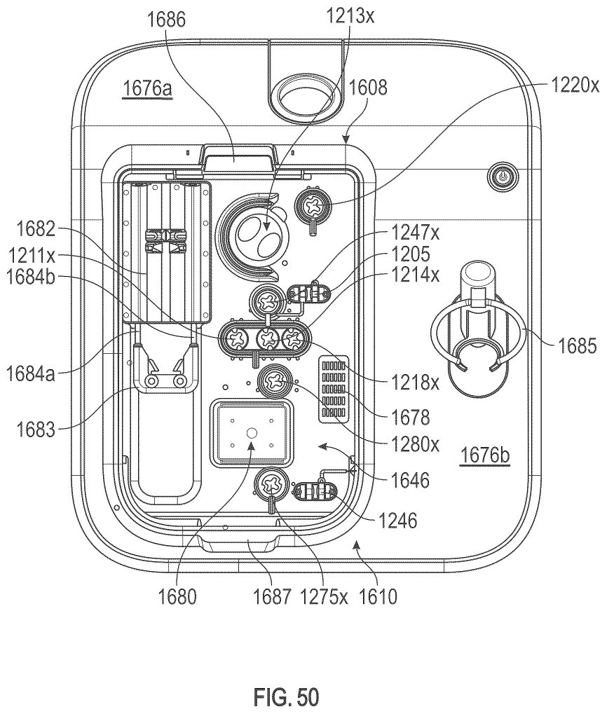

As illustrated in FIG. 39, the drip chamber 1278 and the clot pod 1273 are positioned between the masking 1604 and the proximal side 1612 of the cassette, such that they are visible from the proximal side of the cassette 1200. This position facilitates the drip chamber 1278 and the clot pod 1273 to be imaged by a camera positioned in the interface of the pump station. The control valves 1211, 1214, 1218, 1220, and 1275, and the fluidic channels coupled to the control valves are also visible in this view. In this example, at least a portion of the saline fluidic channel 1206 and a portion of the contrast channel 1244 are held structures (or clips) of the cassette to be apart from surrounding structure to allow air sensors 1205 and 1246 (FIG. 50) to be placed near, or around, a portion of the channels to sense air in the saline fluidic channel 1206 and the contrast fluidic channel 1244 when the cassette 1200 is coupled to the pump station interface.

As illustrated in this embodiment, the electrical interface 1236 is connected to electrical cable 1618 and is positioned on the proximal side of the cassette and includes a plurality of electrical connectors (e.g., pogo pins) that electrically connect to corresponding contacts when cassette is coupled to the pump station to provide signals from the fluidic system (e.g., sensors in the hub assemblies as illustrated in FIG. 19) and the cassette (e.g., pressure sensors 1212, 1283) to the pump station to be processed by a controller.

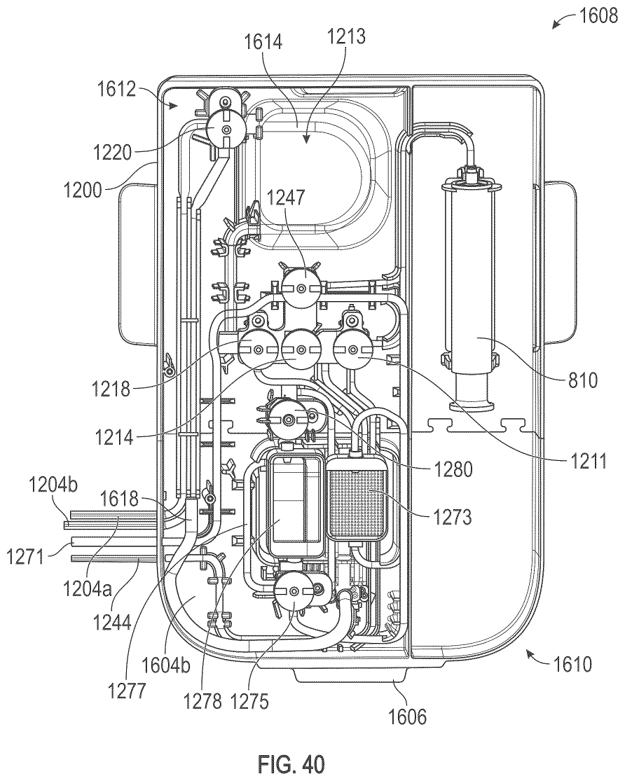

FIG. 40 is another elevation view of the proximal side of the embodiment of the cassette that is illustrated in FIG. 39. In this view although the electrical cable 1618 is shown, the electrical interface 1236 is deliberately not included to illustrate channels that run behind it.

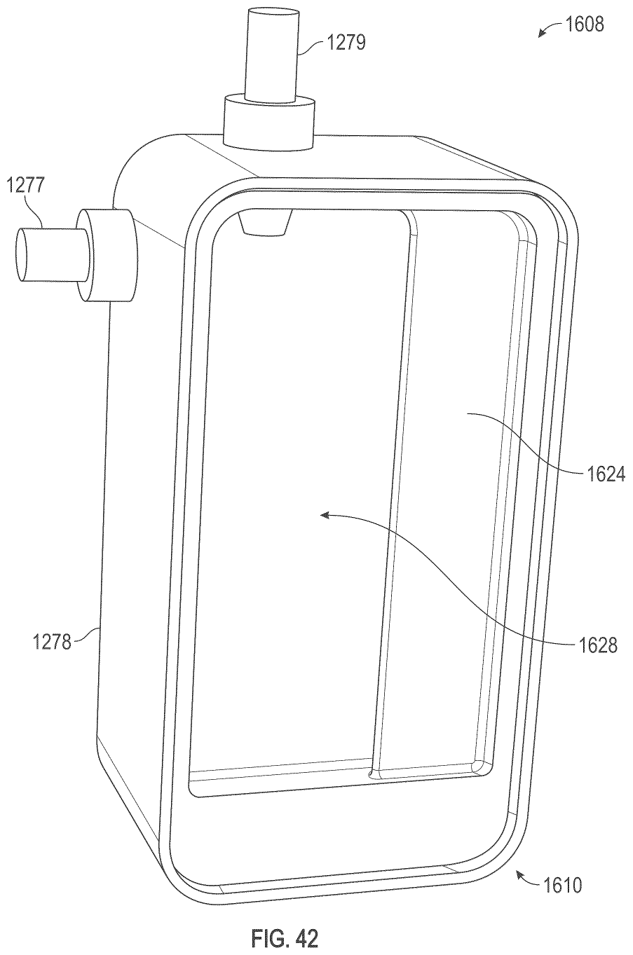

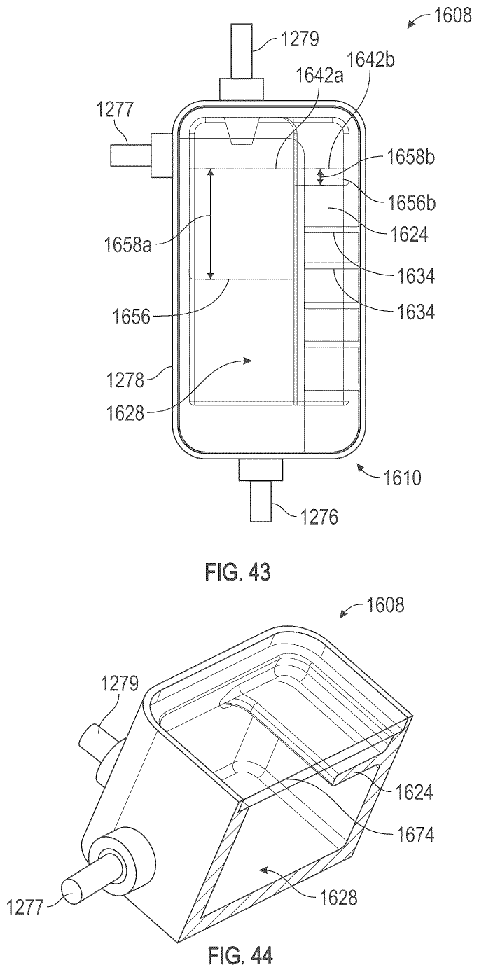

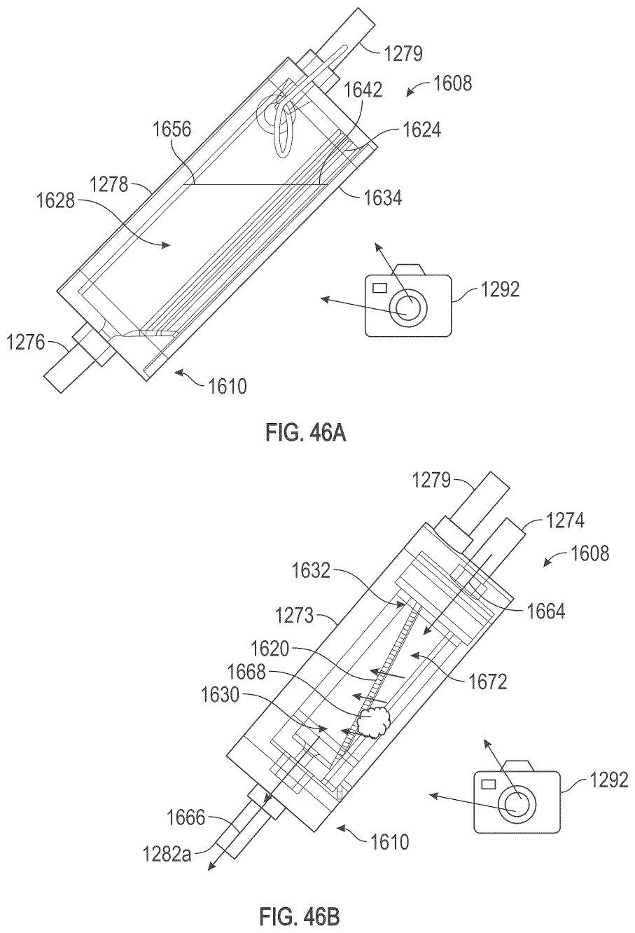

FIGS. 41-47 illustrate various views of an example of a drip chamber 1278 and a clot pod 1273 that can be included a fluidic system and used in various procedures. As an example, for aspiration (e.g., as illustrated of FIGS. 34-38), backbleed (e.g., as illustrated in FIG. 33), and/or patency check of a catheter (e.g., as illustrated in FIG. 37). FIG. 41 illustrates an elevation view of the proximal side of the drip chamber 1278 and the clot pod 1273. When the cassette is mounted to the pump station, the proximal side of the drip chamber 1278 and the proximal side of the clot pod 1273 are oriented to be facing towards the pump station. In this embodiment, the upper portion 1608 of the drip chamber 1278 and the clot pod 1273 are also oriented to be towards an upper portion of cassette 1200 when the cassette is mounted on the pump station, and the lower portion 1610 is oriented to be towards a lower portion of cassette 1200 and the pump station (e.g., similar as depicted in the orientation shown in FIG. 41). In this example, the drip chamber 1278 is coupled to vacuum channels 1276, 1277, and 1279 which are also shown in the fluidic configuration of FIG. 17B. In this embodiment, the drip chamber 1278 includes a main (first) portion 1628 of a first depth and a second portion 1654 of a second depth due to the second portion including a shelf (or rib) 1624 running lengthwise (vertically as oriented in FIG. 41) along an inside side portion of the drip chamber 1278 and extending into the drip chamber 1278, in this example, the right side of the drip chamber 1278 which is located adjacent to the clot pod 1273. In other embodiments, the shelf 1624 can extend from the other side of the drip chamber 1278 (e.g., in FIG. 41, the left-side of the drip chamber) such that the first portion is on the right-side of the drip chamber and the second (shallower) portion is on the left-side of the drip chamber. The shelf 1624 is also illustrated in FIG. 42 which shows a different representation of the drip chamber 1278, showing that the shelf 1624 is located at a different depth than the main portion 1628. For example, in the orientation of FIG. 42, the shelf 1624 is located closer to the proximal side of the drip chamber 1278. The function of the drip chamber is further described below in reference to FIGS. 43 and 46A.

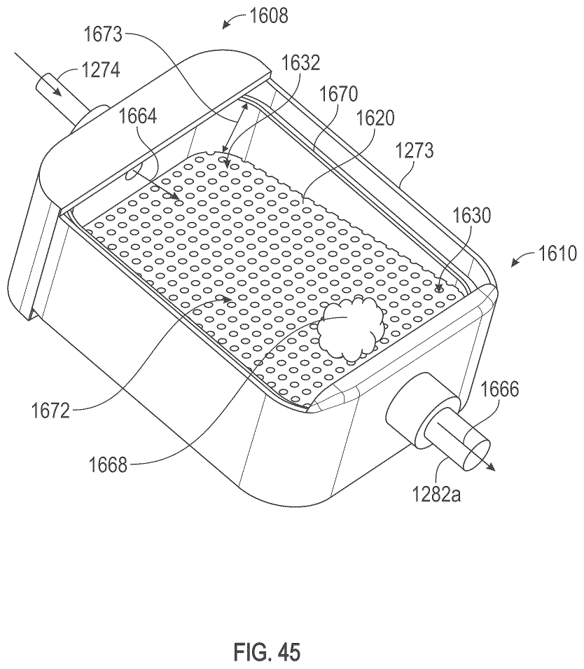

As illustrated in examples herein, the clot pod 1273 can be positioned adjacent to the drip chamber 1278. In some embodiments, the drip chamber 1278 and the clot pod 1273 are coupled together. The clot pod 1273 receives material (e.g., blood, saline, clots) through channel 1274 that is aspirated by the vacuum subsystem, and exhausts liquid to the vacuum cannister via channel 1282a, and is configured to collect non-liquid material (e.g., clot material) and hold it such that it is visible to the camera 1292 (e.g., FIGS. 17B and 46B) for imaging and display. The clot pod 1273 includes one or more screens (filters) 1620. The screen 1620 has a plurality of apertures 1622 that are sized to allow liquid to pass through but are not large enough to allow non-liquid material (e.g., a clot) to pass through. The screen 1620 is positioned at an angle such that an upper portion 1632 of the screen 1620 is positioned further from a proximal edge 1670 than a lower portion 1630 of the screen 1620. In other words, the upper portion of the screen 1632 is set back from the proximal edge 1670 of the clot pod 1273. The set back of the screen 1620 is clearly illustrated in FIG. 45. In this configuration, vacuum can be supplied to the clot pod 1273 through channel 1282a, which is coupled to the clot pod 1273 such that vacuum is supplied to the back (or distal side) of the screen 1620 to create a substance flow from the front (proximal) side of the screen 1620 to the back (distal) side of the screen 1620. Aspirated liquid and material enters the clot pod 1273 through channel 1274 drawn by a vacuum and contacts the proximal side of the screen 1620. Liquid is drawn through the screen 1620, while non-liquid material (e.g., a clot) that is too large to fit through the apertures 1622 remains on the proximal side of the screen 1620 and is typically collects (or is held) on the proximal side of the screen 1620 near the lower portion 1630 of the screen 1630 due to the orientation of the screen 1620 in the clot pod 1273 (see FIG. 45).

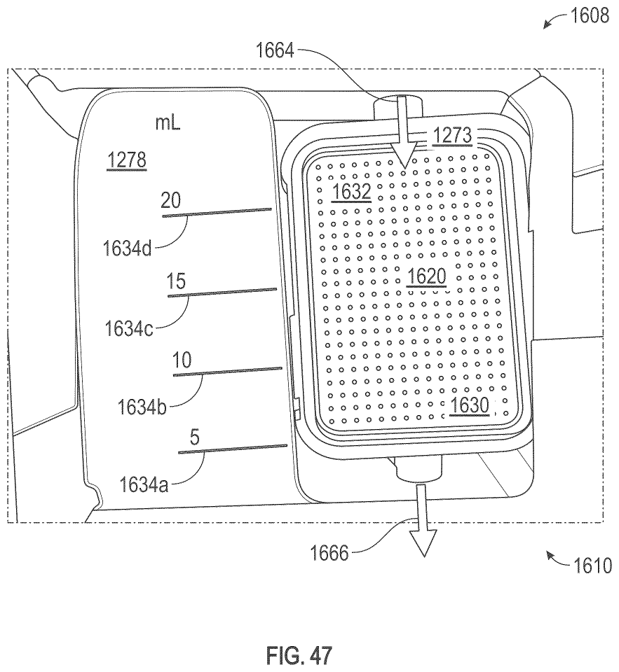

FIG. 43 further illustrates an embodiment of the drip chamber 1278 shown from the proximal side of the drip chamber (i.e., the side of the drip chamber that faces the pump station when the cassette is coupled to the pump station). FIG. 44 illustrates a cutaway view of a perspective view of an upper portion of the drip chamber 1278 shown in FIG. 43, and illustrates position of the shelf 1624 as being located much closer to the proximal side of the drip chamber 1278 compared to the depth of the main compartment 1628. The drip chamber 1278 proximal side can include a transparent wall 1674 through which that a substance (e.g., blood, or saline) in the drip chamber can be viewed either by an imaging system or by a user. The drip chamber 1278 can include one or more markings (or graduations) 1634 that are positioned in front of, or on, the shelf 1624 when viewed from the proximal side of the drip chamber 1278. In an example, the graduations 1634 can be on the transparent wall 1674. In another example, the graduations 1634 can be on another part of the drip chamber, for example, on the shelf 1624. An example of markings 1634 of the drip chamber are illustrated in FIG. 47. In this example, the markings include a 5 mL marking 1634a, a 10 mL marking 1634b, a 15 mL marking 1634c, and a 20 mL marking 1634d, and where the main compartment 1624 of the drip chamber 1278 is masked off. The functionality of the markings 1634 is to indicate the level of liquid in the drip chamber 1278. In this example, the overall drip chamber depth of the drip chamber can be of various depths based on the implementation, however, it can be shallower where the markings 1634 are, which is facilitated by the shelf 1624.

FIG. 46A illustrates a side view of an embodiment of the drip chamber 1278, illustrating an example of the angled position of the drip chamber 1278 when the cassette is coupled to a pump station, and the position of the drip chamber 1278 relative to a camera 1292 positioned in the pump station that the cassette is coupled to. Referring to FIGS. 46A and 46B, when the cassette is coupled to the pump station, the drip chamber 1278 is oriented at an angle with respect to a true vertical orientation, for example, at angle of about 45 degrees. In some embodiments, the angle of the drip chamber 1278 can be between about 10 degrees and 80 degrees, for example, at about 10, 15, 20. 25, 30, 35, 40, 45, 50, 55, 60, 65, 70, 75, or 80 degrees, plus or minus 2.5 degrees. However, in some embodiments, an angle of between about 30 degrees and 60 degrees is preferred as such an angle can make it easier the camera 1292 to accurately image fluid as it drips into the drip chamber 1278. The camera 1292 images the drip chamber 1278 from a position where, due to the angled orientation of the drip chamber when the cassette is coupled to the pump station, the top level (or meniscus) of the liquid appears at two locations. For example, as seen in FIGS. 46A and 46B, the same meniscus appears to have a bottom level 1656 and a top level 1642, where both are actually indicating the top of liquid in the drip chamber 1278, and this can be confusing to an observer (or for software configured for determining the level of liquid in the drip chamber 1278. In FIG. 43, distance 1658a indicates the distance between the representation of the meniscus in the main compartment 1628. Including the shelf 1654 in the drip chamber 1278 and masking off the main compartment 1628 such that the camera 1292 cannot image the lower portion 1656a or the top portion 1642a of the meniscus in the main compartment 1628 helps to mitigate this imaging aberration. The inclusion of the shelf 1624 also mitigates the illusion of having the meniscus appear in two places. FIG. 43 illustrates a representation of the “top” of the meniscus 1642b and the “bottom” of the meniscus as it appears above the shelf 1624, where the distance 1658b between the top representation of the meniscus 1642b and the bottom of the representation of the meniscus 1656b is much smaller. In preferred embodiments, the orientation angle of the drip chamber 1278 in the cassette 1200 when the cassette is coupled to the pump station, and the depth of the shelf 1624 are such that the meniscus of liquid in the drip chamber in the portion above the shelf 1624, and imaged by the camera 1292, appears as a single line, that is, distance 1658b is about zero, or substantially so.

FIG. 45 illustrates a perspective view of the proximal side of an embodiment of a clot pod 1273 illustrated in FIG. 41. In this view, the orientation of the screen 1620 relative to the proximal side of the clot pod 1273 can be clearly seen. On an upper portion 1608 of the clot pod 1273, the upper portion 1632 of the screen 1620 is set back the distance 1673 from a proximal edge 1670 of the clot pod 1273. The distance 1673 is at least an amount such that the upper portion of the screen is set back below clot pod inlet line 1274, and the setback can be substantially the entire depth of the clot pod 1273, or a partial depth of the clot pod 1273. On a lower portion 1610 of the clot pod 1273, the screen 1620 is positioned close to the proximal edge 1670. With this configuration of the screen 1620 in the clot pod 1273, fluid and material flow into the clot pod 1273 via channel 1274 the direction of the fluid and material shown by arrow 1664, the fluid is drawn by vacuum through the screen 1620 exits the clot pod to channel 1282a is illustrated by arrow 1666, while material 1668 (e.g., a clot) that cannot pass through the screen 1620 generally accumulates near the lower portion 1630 of the clot pod 1273 instead of being located anywhere in the clot pod. Advantageously, this can allow for a quicker assessment of having received a clot in the clot pod, by a user viewing the images generated by the camera 1292 on a display located remotely and/or locally, and/or by software analyzing the images generated by the camera in real time. FIG. 47 illustrates an example of an image of the clot pod 1273 generated by the camera 1292. FIG. 46B further illustrates the configuration of the screen 1620 in the clot pod 1273, showing a side view of an embodiment of a clot pod 1273 positioned near a camera 1292, and showing the clot 1668 caught in the lower portion 1630 of the clot pod chamber 1672.

FIG. 48 illustrates an example of a pump station 1650, and a cassette 1200 coupled to an interface 1646 of the pump station 1650. FIGS. 49-60 further illustrate aspects of the pump station interface 1646. The pump station 1650 includes a housing 1651 that encloses other components of the pump station. In this example, the pump station 1650 includes a display 1648 positioned on an upper portion 1608 of the pump station 1650 above an interface surface 1676. The interface 1646 forms a portion of the interface surface 1676 and is further described below. The proximal side of the cassette 1200 is coupled to the interface 1646, with an upper portion 1608 of the cassette 1200 coupled to an upper portion of the interface 1646, in a lower portion of the cassette 1610 coupled to a lower portion of the interface 1646. A contrast bottle 1108 can be coupled to the interface surface 1676 to hold the contrast bottle in a position above or at the same level as the cassette 1200 when the cassette is coupled to the interface 1646.

FIG. 49 illustrates a perspective front view of an example of the pump station interface 1646 which includes components that are configured to interact with corresponding components on the cassette, when the cassette is coupled to the interface 1646. FIG. 50 illustrates an elevation front view of the example of the pump station interface shown in FIG. 49. In this example, the interface includes a rotational portion of the peristaltic pump 1213x, that interacts with a portion of the peristaltic pump 1213 of the cassette. The interface 1646 also includes control valves valve actuators that are configured to actuate control valves in the cassette 1200. For example, actuator 1211x that is configured to couple to and move the contrast refill stopcock 1211, control valve actuator 1214x that is configured to couple to and move the saline primed stopcock 1214, control valve actuator 1218x that is configured to couple to and move the saline bypass stopcock 1218, control actuator 1280x that is configured to couple to and move the control valve 1280, control valve actuator 1275x that is configured to couple to a move control valve 1275 of the vacuum subsystem 1203, and control valve actuator 1220x that is configured to couple to and move the saline source selector control valve 1220.

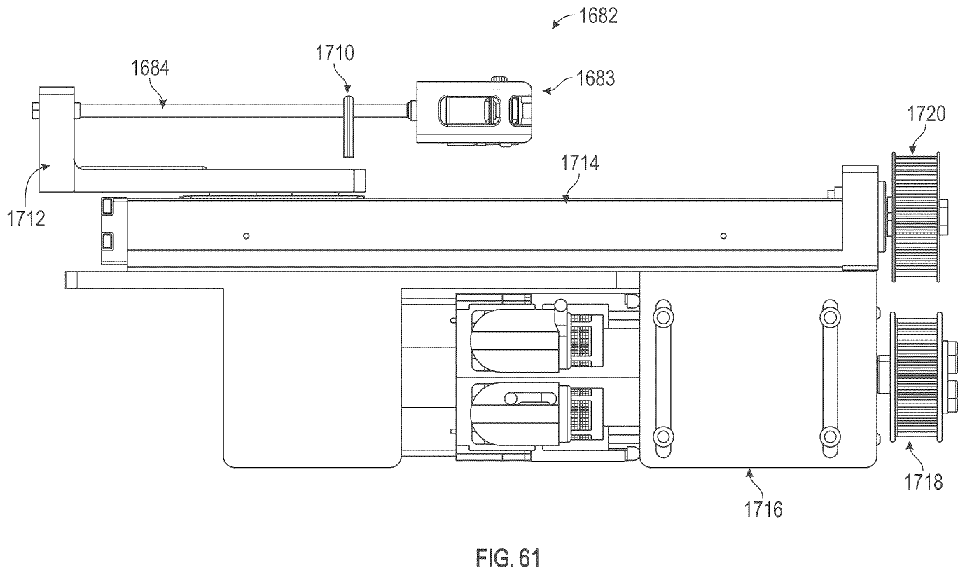

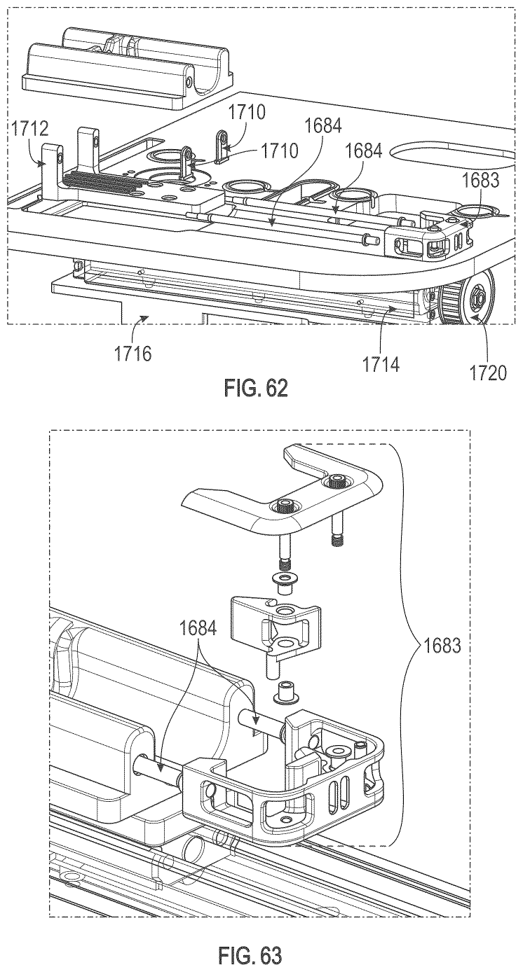

The interface 1646 further includes contrast pump actuator assembly 1682 that is configured to receive and couple to the contrast pump 810 when the cassette is coupled to the interface 1646. The contrast pump actuator assembly 1682 includes a contrast pump (syringe) latch 1683 that is configured to couple to a proximal portion of the contrast pump 810, and be moved by piston rods 1684 (e.g., rods 1684a-b) to fill the contrast pump 810 with contrast when moved in one direction, and to provide contrast under high pressure, through the fluidic system to one or more catheters coupled to hub assemblies of the robotic catheter system, when moved in the opposite direction. FIGS. 61, 62, and 63 provide additional views of the contrast pump actuator assembly 1682. For example, FIG. 61 illustrates a side elevation view of an example of the contrast pump actuator assembly 1682, which includes a motor 1716 which is positioned in the pump station below the pump station interface 1646. The motor includes a first pulley 1718 that can be coupled to a second pulley 1720 of the monocarrier 1714, for example, with a drive belt. The contrast pump actuator assembly 1682 also includes an inverting bracket 1712 coupled to the monocarrier 1714, and piston rods 1684 coupled to a vertical section of the inverting bracket 1712. The piston rods 1684 extend through a seal 1710 and are coupled to the syringe latch 1683. FIG. 62 illustrates a side perspective view of these components view of the contrast pump actuator assembly illustrated in FIG. 63 illustrates an exploded view of the embodiment of the contrast pump actuator latch 1683 illustrated in FIGS. 61 and 62. This contrast pump actuator latch 1683 configuration, where the pistons 1684 are used to pull the latch 1683 to actuate the contrast pump 810 to provide contrast at high pressure (e.g., 200-400 PSI) advantageously provides a compact actuator for the contrast pump 810 when compared to configurations that use a piston positioned at the proximal end of the contrast pump 810 to actuate the contrast pump through a pushing force, which can require 2-3 times the actuation travel distance for the configuration. In addition, a large “pulling” force can be exerted on the piston rods 1684 when actuating the contrast pump 810 which will not bend the pistons rods 1684, which provides an advantage to configurations where one or more pistons rods are pushed with a large force to actuate the contrast pump which can result in bending the piston rods, or having to use larger piston rods that will not bend.

The interface 1646 further includes an electrical interface 1678 that is configured to electrically couple to the electrical interface 1236 of the cassette 1200. In this embodiment, the electrical interface 1678 includes a plurality conductive surfaces that each can be electrically connected to a pogo pin on the electrical interface 1236. The interface 1646 also includes the camera portion 1680 that contains an imaging device configured to generate images of the drip chamber and clot pod. The interface 1646 also includes air sensors 1205 and 1246 that are configured to be positioned adjacent to the saline channel 1206 and the contrast channel 1244, respectively, to sense the presence of air in these channels when the cassette 1200 is coupled to the interface 1646. Other components of the interface 1646 include a lower cassette connector 1687 which couples to a lower portion of the cassette 1200, and in upper cassette connector 1686 which couples to an upper portion of the cassette 1200, when the cassette is coupled to the interface 1646. This example of the pump station also includes a contrast bottle holder 1685 coupled to the pump station surface 1676. Also in this example, the pump station surface includes an upper portion 1676a that is aligned to be generally horizontal, and a lower portion 1676b that is aligned at an angle with respect to the upper portion 1676a, such that when the cassette 1200 is coupled to the interface 1646, cassette 1200 is also oriented at an angle.

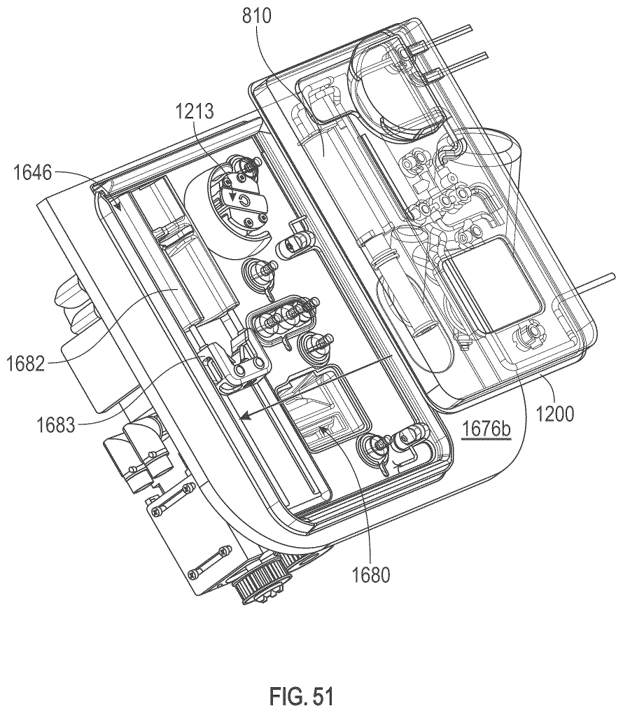

FIG. 51 illustrates a perspective front view of an example of a pump station interface 1646 and an example of a cassette 1200 positioned above the pump station interface 1646 to illustrate their respective positions prior to being coupled together. In certain configurations, to couple the cassette 1200 to the pump station interface 1646, the cassette 1200 is generally aligned to be parallel to the pump station interface 1646 and then moved towards the interface 1646 too carefully couple the contrast pump 810 into the contrast pump actuator assembly 1682 and such that the control valve actuators on the pump station interface 1646 aligned with coupled to the control valves in the cassette 1200. Depending on the particular implementation, the cassette can be coupled to the interface 1646 by structure on a lower portion of the interface 1646 and the cassette 1200 and by structure on an upper portion of the interface 1646 and the cassette 1200.

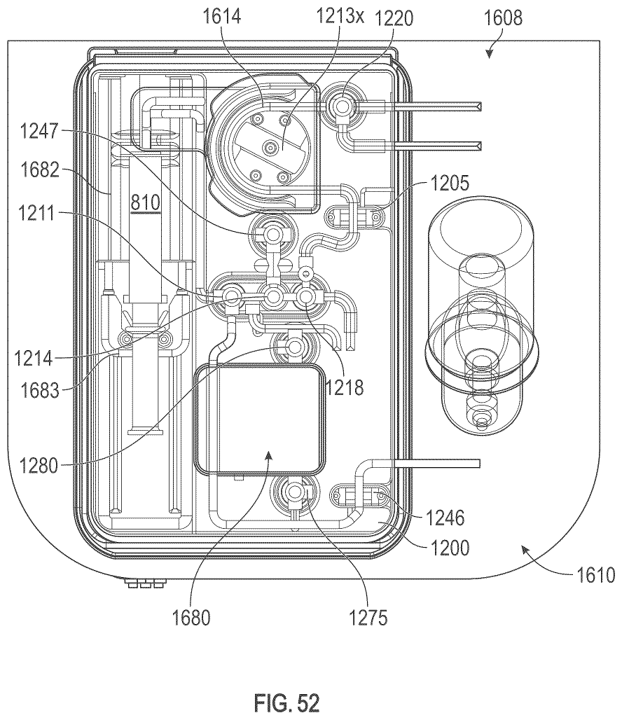

FIG. 52 is a representation of a front ghost view of an example of an example of a cassette 1200 coupled to an embodiment of a pump station interface 1646. In this representation, the contrast pump (syringe) 810 is shown coupled to the contrast pump actuator assembly 1682, and the syringe latch 1683 is coupled to the proximal portion of the contrast pump 810. The portion of the peristaltic pump 1213x is shown to be coupled to saline channel 1614 of the cassette. The control valve actuators of the interface 1646 are coupled to the control valves of the cassette, and the air sensors 1205 and 1246 of the interface 1646 are coupled to a saline channel and a contrast channel, respectively, of the cassette 1200.

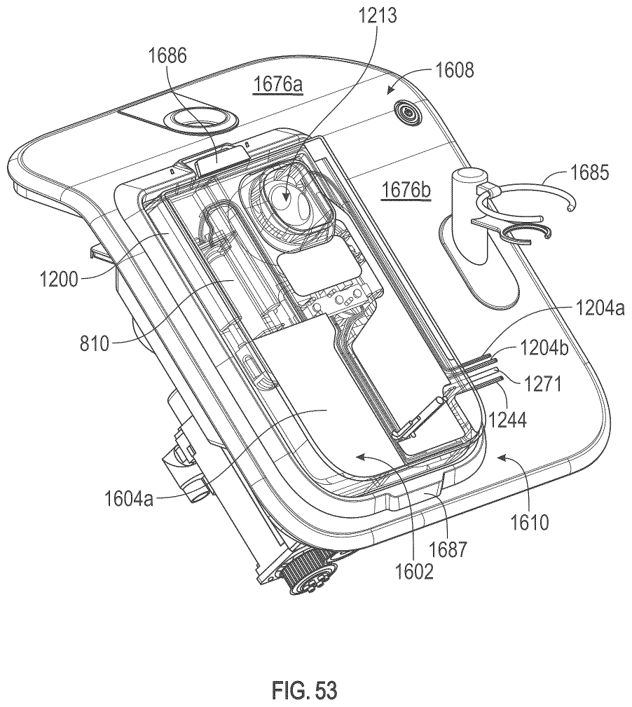

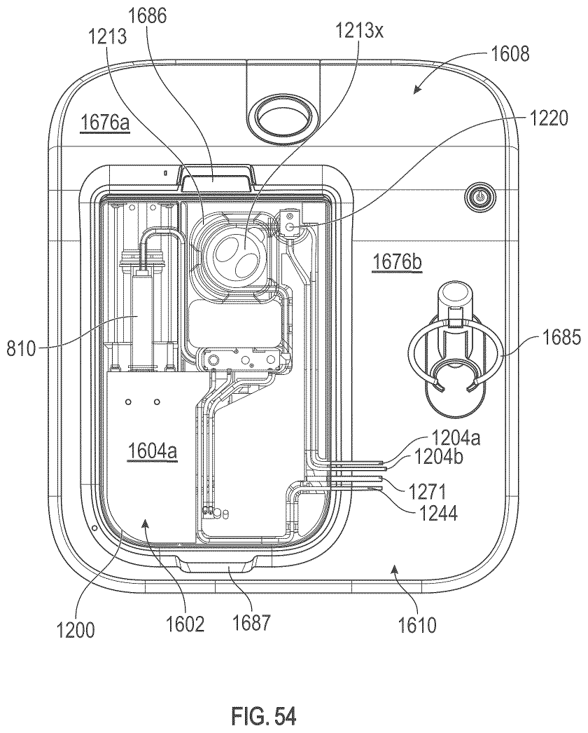

FIG. 53 is a perspective front ghost view of an example of a cassette 1200 coupled to an embodiment of a pump station interface 1646, illustrating a representation of a view of the cassette 1200 with masking 1604 covering certain components of the cassette 1200. FIG. 54 is an elevation front view of an example of a cassette coupled to an embodiment of a pump station interface 1646, the distal side 1602 of the cassette 1200 facing away from the pump station interface 1646. It is noted that the input lines for saline 1204, contrast 1244, and aspiration 1271 are illustrated in particular exemplary configuration, however, other configurations of input lines from the saline, contrast, and vacuum sources into the cassette 1200, and output lines from the cassette 1200 to provide saline, contrast, and vacuum downstream to the hub assemblies and catheters, are also possible.

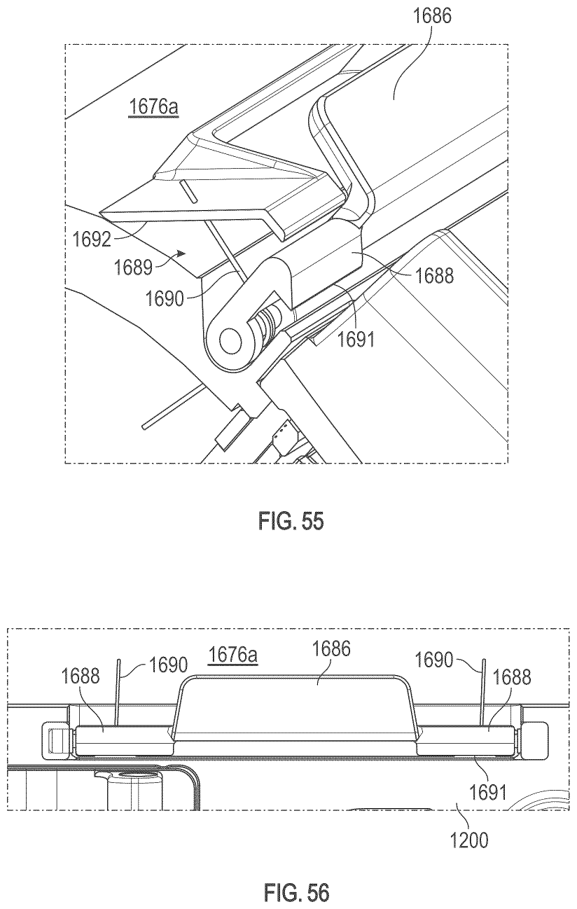

FIG. 55 is a perspective view of an example of a portion of a latch assembly 1689 on the pump station that is used to couple a cassette 1200 to a pump station interface 1646, FIG. 56 illustrates another view of the latch assembly 1689 shown in FIG. 55. The latch assembly 1689 is positioned on the upper end of the pump interface 1646, and includes a handle 1686 that is coupled to movable clips 1688, and one or more springs 1690 that are used to bias the clips 1688 to position an edge 1691 of the clips 1688 to attach to the upper portion of the cassette 1200 and hold the cassette 1200 onto the interface 1646. In this embodiment, handle 1686 can be pushed towards the pump station to lift the clips to allow the cassette to be placed on the interface 1646 and then the handle can be released or pulled away from the pump station to secure the cassette to the pump station. The latch assembly can also include a clip cover 1692 that extends over the clips 1688 and secure the clips in a locked position when they are holding a cassette on the interface 1646. In other embodiments, other configurations of one or more clips can be used to hold the cassette 1200 on the pump interface 1646. For example, in the illustrated embodiment includes a handle centrally located at the top of the interface. In other embodiments, other configurations of latch assemblies can be used, and the motion to latch or unlatch latch assembly may be linear motion.

FIG. 57 illustrates an example of a lower portion of a pump station interface (“interface”) 1640 configured with a recess 1687 on a lower portion 1610 of the interface 1646. The recess is size and shaped to receive structure 1694 (FIG. 58) extending from the proximal side 1612 of the cassette 1200 when the cassette 1200 is coupled to the interface 1646. In this embodiment, a portion of the cassette 1200c extends behind a portion of the pump station interface 1646c when the structure 1694 is received in the recess 1687, similar to a tongue-and-groove configuration. The structure 1694 advantageously clearly indicates the orientation of the cassette 1200 (i.e., what side of the cassette 1200 attaches to the lower portion of the interface 1646 and helps to properly roughly align the cassette 1200 to the interface 1646, and helps to prevent the cassette 1200 from moving downward on the interface 1646.

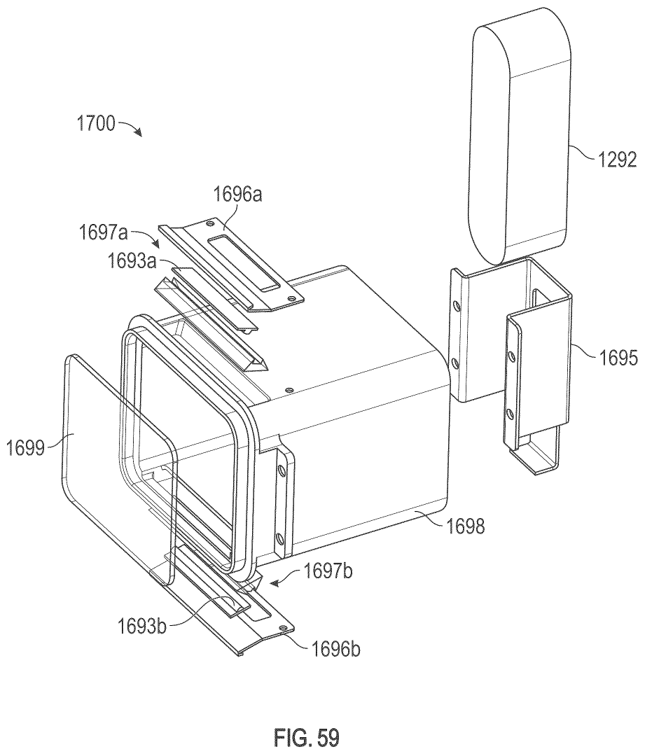

FIG. 59 illustrates an example of a camera assembly 1700 that is positioned in the pump station interface 1646 in a location to image a drip chamber 1278 and a clot pod 1273 positioned in the cassette 1200. The camera assembly 1700 includes camera 1292 in the bracket 1695 configured to hold the camera 1292. Camera assembly 1700 further includes a housing 1698, a transparent cover 1699, and one or more light assemblies 1697, the illustrated embodiment including two light assemblies 1697a and 1697b. Each light assembly 1697 can include a light source 1693 (e.g., light sources 1693a-b) and a bracket 1696 (e.g., brackets 1696a-b) which holds the light source 1693 in a position to illuminate the drip chamber 1278 and the clot pod 1273 when the cassette 1200 is coupled to the pump station interface 1646. The light source 1693 be in the late source, for example, an LED light source. The camera 1292 can be configured to have a depth of field of sufficient depth to image the drip chamber 1278 and the clot pod 1273 without having to focus the camera 1292. The camera 1292 can be configured to generate single images as needed, or to generate a series of images (e.g., video), which can then be displayed on a local or a remotely located display, and/or automatically processed to determine presence of a clot in the clot pod and/or the flow of liquid into the drip chamber and the level of liquid of the drip chamber.

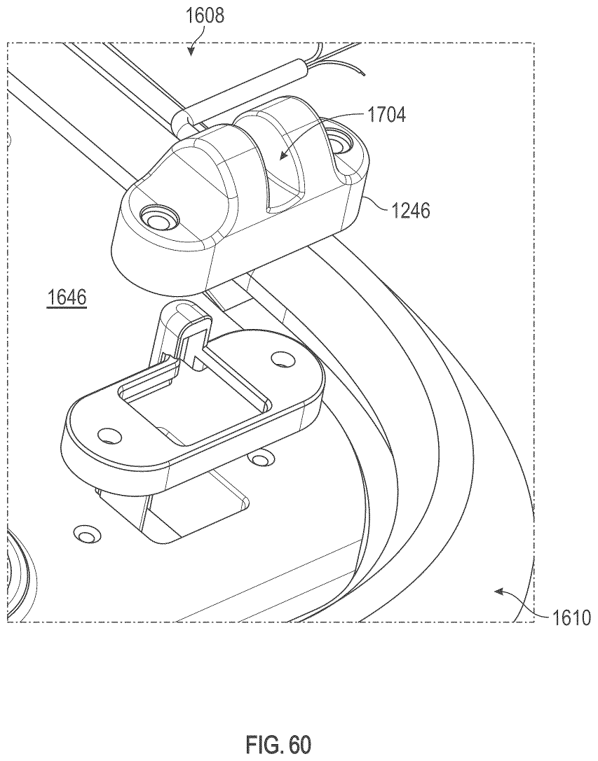

FIG. 60 illustrates an example of an air sensor positioned in a pump station interface and configured to sense air in a channel positioned in a portion of the air sensor. In this example, air sensor 1246 is positioned to receive, in aperture 1704, a portion of the contrast channel 1244 (of the cassette) when the cassette 1200 is coupled to the pump station interface 1646. This configuration, where the air sensors are part of the pump station interface 1646, allows the air sensors 1246 to be part of the capital equipment instead of being disposable.

Various fluidics systems and methods are described herein primarily in the context of providing saline, contrast, and vacuum to an interventional device. However, in certain embodiments, other fluids, such as one or more drugs or components thereof (e.g., therapeutic drugs, anti-complication drugs, lytic drugs, non-lytic drugs, post thrombectomy drugs, interventional oncology drugs), can be provided using the fluidics systems and/or methods described herein. For example, a drug may be delivered through an interventional device alternatively to saline, contrast, and/or vacuum or additionally to saline, contrast, and/or vacuum using a subsystem that is the same as or generally similar to the saline subsystem or the contrast subsystem.

The systems and methods of the preferred embodiment and variations thereof can be embodied and/or implemented at least in part as a machine configured to receive a computer-readable medium storing computer-readable instructions. The instructions are preferably executed by computer-executable components preferably integrated with the system and one or more portions of the processor on a hubs, RHVs, and/or a computing device associated with the fluidics management systems described herein. The computer-readable medium can be stored on any suitable computer-readable media such as RAMs, ROMs, flash memory, EEPROMs, optical devices (e.g., CD or DVD), hard drives, floppy drives, or any suitable device. The computer-executable component is preferably a general or application-specific processor, but any suitable dedicated hardware or hardware/firmware combination can alternatively or additionally execute the instructions.

Various systems and methods are described herein primarily in the context of a neurovascular access or procedure. However, the inventors contemplate applicability of the disclosed catheters, systems and methods to any of a wide variety of alternative applications, including within the coronary vascular or peripheral vascular systems as well as other hollow organs or tubular structures in the body.

As used in the description and claims, the singular form “a”, “an” and “the” include both singular and plural references unless the context clearly dictates otherwise. At times, the claims and disclosure may include terms such as “a plurality,” “one or more,” or “at least one;” however, the absence of such terms is not intended to mean, and should not be interpreted to mean, that a plurality is not conceived.

The term “about” or “approximately,” when used before a numerical designation or range (e.g., to define a length or pressure), indicates approximations which may vary by (+) or (−) 5%, 1% or 0.1%. All numerical ranges provided herein are inclusive of the stated start and end numbers. The term “substantially” indicates mostly (i.e., greater than 50%) or essentially all of a device, substance, or composition.

As used herein, the term “comprising” or “comprises” is intended to mean that the devices, systems, and methods include the recited elements, and may additionally include any other elements. “Consisting essentially of” shall mean that the devices, systems, and methods include the recited elements and exclude other elements of essential significance to the combination for the stated purpose. Thus, a system or method consisting essentially of the elements as defined herein would not exclude other materials, features, or steps that do not materially affect the basic and novel characteristic(s) of the claimed disclosure. “Consisting of” shall mean that the devices, systems, and methods include the recited elements and exclude anything more than a trivial or inconsequential element or step. Embodiments defined by each of these transitional terms are within the scope of this disclosure.

The examples and illustrations included herein show, by way of illustration and not of limitation, specific embodiments in which the subject matter may be practiced. Other embodiments may be utilized and derived therefrom, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is in fact disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. A fluidics system, comprising:
a cassette having a housing with a proximal surface structured to be releasably coupled to a pump station, the cassette including:
a vacuum subsystem having a vacuum flow-path for communicating aspirated material, the vacuum subsystem including
a drip pod positioned in the vacuum flow-path, the drip pod including
a drip chamber structured to allow observation of an amount of fluid being communicated through the vacuum flow-path, and
a clot pod coupled to the drip chamber and structured to collect material communicated through the vacuum flow-path,
wherein the drip pod includes a transparent wall adjacent to the cassette proximal surface for allowing an imaging system to image material in the drip chamber and the clot pod through the transparent wall.

2. The fluidics system of claim 1, wherein the clot pod includes an inlet port and an outlet port, and a screen positioned between the inlet port and outlet port for collecting material communicated through the clot pod.

3. The fluidics system of claim 2, wherein the screen is positioned at an angle in the clot pod such that a first portion of the screen is farther from the proximal surface and a second portion of the screen is located closer to the proximal surface.

4. (canceled)

5. The fluidics system of claim 2, further comprising the pump station, wherein the pump station and the cassette are configured such that the drip pod is positioned at an angle of between about 30 degrees and about 80 degrees above horizontal when the proximal surface of the cassette is coupled to the pump station.

6. (canceled)

7. The fluidics system of claim 1, wherein the cassette is configured such that the drip pod is positioned at an angle of about 45 degrees above horizontal, when the proximal surface of the cassette is coupled to the pump station.

8. The fluidics system of claim 1, wherein the drip chamber comprises a first portion and a second portion, the second portion being shallower than the first portion.

9. The fluidics system of claim 8, wherein the second portion is defined by a shelf extending from a side of the drip chamber into the drip chamber to create the second portion of the drip chamber.

10. The fluidics system of claim 8, wherein the drip chamber further comprises graduations disposed between the second portion of the drip chamber and the transparent wall, the graduations indicative of a level of fluid in the drip chamber.

11. The fluidics system of claim 1, wherein the cassette includes a proximal side that includes the proximal surface and a distal side, opposite the proximal side, the cassette further including masking positioned to obscure a portion of components inside the cassette when the cassette is viewed from its distal side.

12. The fluidics system of claim 1, wherein the cassette includes a contrast pump configured to interface with a contrast pump actuator on the pump station when the cassette is coupled to the pump station.

13. The fluidics system of claim 1, wherein the cassette further includes a saline subsystem and a contrast system, each coupled to the vacuum subsystem, the saline and contrast subsystems configured to selectively communicate saline and contrast to the vacuum subsystem.

14. A fluidics system, comprising:
a pump station interface comprising a contrast pump actuator, a camera, a peristaltic pump actuator, and an electrical interface; and
a cassette having a distal side and a proximal side, the proximal side of the cassette configured to releasably couple to the pump station interface, the cassette comprising a contrast pump, a saline channel configured to interface with the peristaltic pump actuator, and a drip chamber and clot pod positioned in the cassette to be adjacent to the camera when the cassette is coupled to the interface.

15. The fluidics system of claim 14, wherein the drip chamber comprises a main compartment and a shelf extending from the side of the drip chamber into the main compartment to create a shallower portion of the drip chamber adjacent to the proximal side of the cassette.

16. The fluidics system of claim 14, wherein the drip chamber further comprises graduations indicative of a level of fluid in the drip chamber.

17. The fluidics system of claim 14, further comprising a drip pod comprising the drip chamber and the clot pod, wherein the drip pod is positioned at an angle of between about 30 degrees and about 60 degrees relative to vertical when the cassette is coupled to the pump station interface.

18. (canceled)

19. The fluidics system of claim 14, wherein the clot pod includes a screen positioned at an angle within the clot pod to form a clot collection portion of the clot pod that is shallower on one side of the clot pod than the other side of the clot pod.

20. The fluidics system of claim 19, wherein clot pod is configured such that the clot collection portion of the clot pod is shallower on a lower portion of the clot pod than a upper portion of the clot pod when the cassette is coupled to the pump station interface.

21. The fluidics system of claim 14, wherein at least a portion of the proximal side of the cassette positioned adjacent to the drip chamber and clot pod is transparent.

22. (canceled)

23. The fluidics system of claim 14, wherein the contrast pump actuator is configured to receive the contrast pump when the cassette is coupled to the pump station interface, and wherein the contrast pump actuator includes at least one piston rod attached to a latch assembly that couples to a portion of the contrast pump, the contrast pump actuator configured to actuate the contrast pump to provide contrast at high pressure to a contrast flowpath by pulling the latch with the at least one piston rod.

24. The fluidics system of claim 14, wherein the pump station interface further comprises a plurality of control valve actuators, and wherein the cassette further comprises a plurality of control valves positioned to correspond the plurality of control valve actuators, wherein the plurality of control valves are configured to selectively control the flow of contrast from the contrast pump through the cassette and selectively control the flow of saline through the saline channel of the cassette.

25.-38. (canceled)
